(19) United States
(12) Patent Application Publication
(10) Pub. No.: US 2024/0327789 A1
(43) Pub. Date: Oct. 3, 2024

(54) HUMAN CEREBELLAR ORGANOIDS WITH BONA FIDE PURKINJE CELLS AND USES THEREOF

(71) Applicant: University of Southern California, Los Angeles, CA (US)

(72) Inventors: Alexander Atamian, Los Angeles, CA (US); Giorgia Quadrato, Los Angeles, CA (US)

(73) Assignee: University of Southern California, Los Angeles, CA (US)

(21) Appl. No.: 18/625,740

(22) Filed: Apr. 3, 2024

Related U.S. Application Data
(60) Provisional application No. 63/456,584, filed on Apr. 3, 2023.

(51) Int. Cl. C12N 5/0793; C12N 5/00; G01N 33/50

(52) U.S. Cl. CPC C12N 5/0619; C12N 5/0018; G01N 33/5014; G01N 33/5023; G01N 33/5058; G01N 33/5088; C12N 2500/25; C12N 2500/40; C12N 2500/44; C12N 2501/119; C12N 2501/13; C12N 2501/21; C12N 2501/395; C12N 2501/91; C12N 2501/999; C12N 2503/02; C12N 2506/02; C12N 2506/45; C12N 2513/00; C12N 2527/00

(57) ABSTRACT

We report human cerebellar organoids which recapitulate the major milestones of cerebellar development including generating bona fide Purkinje cells, as well as methods of making and using these human cerebellar organoids such as uses in drug target identification and drug screening. In various embodiments, we report a human organoid model (human cerebellar organoids [hCerOs]) capable of developing the complex cellular diversity of the fetal cerebellum, including a human-specific rhombic lip progenitor population that have never been generated in vitro prior to our study. 2-month-old hCerOs form distinct cytoarchitectural features, including laminar organized layering, and create functional connections between inhibitory and excitatory neurons that display coordinated network activity. Long-term culture of hCerOs allows healthy survival and maturation of Purkinje cells that display molecular and electrophysiological hallmarks of their in vivo counterparts, addressing a long-standing challenge in the field.

### CROSS-REFERENCE TO RELATED APPLICATIONS

This application includes a claim of priority under 35 U.S.C. § 119(e) to U.S. provisional patent application No. 63/456,584, filed Apr. 3, 2023, the entirety of which is hereby incorporated by reference.

### REFERENCE TO SEQUENCE LISTING

This application contains a Sequence Listing submitted as an electronic file named “065715_000154USPT_SequenceListing.xml”, having a size in bytes of 18,728 bytes, and created on Apr. 2, 2024 (WIPO production date noted as Apr. 2, 2024). The information contained in this electronic file is hereby incorporated by reference in its entirety.

### FIELD OF INVENTION

This invention relates to human stem cell cultivation, and specifically the generation and use of cerebellar-specific, human cell-based organoids.

### BACKGROUND

The cerebellum has been gaining substantial attention, given emerging evidence of its role in cognitive functions, including language, spatial processing, working memory, executive functions, and emotional processing, in addition to its well-described role in motor behaviors. In all mammals, the cerebellum is initially derived from the alar lamina (dorsal) rhombomere 1 (r1) region caudal to the isthmic organizer at the midbrain-hindbrain boundary. The r1 comprises two distinct progenitor zones including the PTF1A+/KIRREL2+ ventricular zone (VZ) and the ATOH1+ Rhombic Lip (RL). The VZ produces GABAergic neurons, including Purkinje Cells, molecular layer interneurons (MILIs), and deep cerebellar nuclei (inhibitory cerebellar nuclei [iCNs]), whereas the more dorsally located RL gives rise to glutamatergic neurons, including granule cells (GCs), unipolar brush cells (UBCs), and large deep cerebellar nuclei projection neurons (excitatory cerebellar nuclei [eCNs]).

Despite the conservation of early developmental stages of cerebellar ontogenesis across species, human-specific traits, including altered neuronal subtype ratios and increased folial complexity, have been identified when compared with non-human mammalian counterparts. In addition, comparison between human and non-human primates has identified the presence of a human-specific population of neural progenitors derived from the rhombic lip. This is important, as abnormal development of the human rhombic lip is associated with multiple cerebellar disorders, including Dandy-Walker malformation, cerebellar vermis hypoplasia, and medulloblastoma, the most common metastatic childhood brain tumor. Other cerebellar disorders, including autism spectrum disorder (ASD) and ataxia, have been associated with the degeneration of Purkinje cells, the main output neurons of the cerebellar cortex. These disorders have mainly been studied in mouse models, which cannot fully recapitulate human disease phenotypes. An example of this includes the inability of mice to recapitulate the Purkinje cell loss phenotype observed in patients with ataxia telangiectasia. Thus, there is a pressing need to develop an all-human cell-based culture system capable of generating functional Purkinje cells.

Thus, it is an objective of the present invention to provide an all-human cell-based culture system capable of generating bonafide Purkinje cells.

It is another objective of the present invention to provide methods of producing all-human cell-based, cerebellar organoid culture system, and methods of using them.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Methods for generating a cerebellum-like organoid from human stem cells are provided, which include the generation of mature, functional Purkinje cells that are in various aspects positive for PCP2, DAB1, RORA, and FOXP2 and characterized with hyperpolarization-activated current and repetitive spontaneous firing.

In various embodiments, the methods for generating a cerebellum-like organoid include all, or some steps if starting from an intermediate state of cell types other than human stem cells, of:
a) culturing human stem cells in the presence of two or more SMAD signaling inhibitors, a glycogen synthase kinase 3 (GSK3) inhibitor, and optionally a rho-associated, coiled-coil containing protein kinase (ROCK) inhibitor (ROCKi) in a growth factor-reduced medium to obtain neuronal lineage embryoid bodies (EBs);
b) culturing the neuronal lineage EBs in the presence of a midbrain-hindbrain morphogen and the two or more SMAD signaling inhibitors and the GSK3 inhibitor in the growth factor-reduced medium, thereby obtaining midbrain-hindbrain regionalized tissues;
c) culturing the midbrain-hindbrain regionalized tissues in the presence of the midbrain-hindbrain morphogen, the two or more SMAD signaling inhibitors, and the GSK3 inhibitor in a first cerebellar differentiation medium (CerDM1), thereby obtaining isthmic organizer regionalized tissues;
d) culturing the isthmic organizer regionalized tissues in a second cerebellar differentiation medium (CerDM2) in motion, in an agitated environment, or on an orbital shaker, thereby obtaining cerebellar neuroepithelial tissues, wherein the CerDM2 does not contain the one or more SMAD signaling inhibitors, the GSK3 inhibitor, the ROCKi, and the midbrain-hindbrain morphogen; and
e) culturing the cerebellar neuroepithelial tissues in the presence of a thyroid hormone and optionally further in presence of one or more of a solubilized basement membrane matrix, stromal cell-derived factor 1 alpha (SDF1a), and brain-derived neurotrophic factor (BDNF) in a third cerebellar differentiation medium (CerDM3) thereby forming a cerebellum-like organoid, wherein the cerebellum-like organoids contain mature, functional Purkinje cells.

In preferred embodiments, the methods of generating cerebellum-like organoid provided herein do not include culturing in the presence of mouse granule cells or mouse glial cells.

In some embodiments, the two or more SMAD signaling inhibitors comprise SB431542 and noggin. In some embodiments, the GSK3 inhibitor comprises CHIR99021. In some embodiments, the ROCKi comprises Y-27632. In some embodiments, the midbrain-hindbrain morphogen comprises fibroblast growth factor 8b (FGF8b). In further embodiments, the step c) includes using the FGF8b at a first concentration (e.g., 100 ng/mL) in a first period of time, followed by using the FGF8b at a second concentration (e.g., 300 ng/mL) higher than that of the first concentration in a second period of time. In some embodiments, the thyroid hormone comprises T3.

In some embodiments, the growth factor-reduced medium is a growth factor-reduced chemically-defined medium (gfCDM) comprising a mixture of Iscove's Modified Dulbecco's Medium (IMDM) and Ham's F-12 nutrient mix (F-12), supplemented with bovine serum albumin, a chemically defined lipid concentrate (CDLC), apo-transferrin, mono-thioglycerol, and insulin, and lacking a growth factor. In some embodiments, the CerDM1 comprises a mixture of Dulbecco's Modified Eagle Medium (DMEM) and F-12, supplemented with knockout serum replacement (KSR), apo-transferrin, insulin, glutamax (L-alanyl-L-glutamine dipeptide), and 2-mercaptoethanol. In some embodiments, the CerDM2 comprises a mixture of DMEM and F-12, supplemented with N-2 supplement, B-27, and glutamax (L-alanyl-L-glutamine dipeptide). In some embodiments, the CerDM3 comprises a mixture of DMEM, F-12, and neurobasal medium, supplemented with N-2 supplement, B-27, glutamax (L-alanyl-L-glutamine dipeptide), heparin, CDLC, and amphotericin B.

In some embodiments, the culturing in step b) comprises culturing for about 4 days or between 3 and 6 days. In some embodiments, the culturing in step c) comprises culturing for about 11 days or between 9 and 13 days. In some embodiments, the culturing in step d) comprises culturing for about 13 days or between 10 and 15 days. In some embodiments, the culturing in step e) comprises culturing for about 30 days or between 20 and 40 days in the presence of the thyroid hormone, the solubilized basement membrane matrix, and the SDF1a, and optionally further comprising subsequent culturing in the presence of the BDNF after the about 30 days or the between 20 and 40 days.

In various aspects, the cerebellum-like organoids each contain ventricular zone progenitor cells, rhombic lip progenitor cells, a cluster of Bergmann glial marker-positive cells, a cluster of neuronal cells, a cluster of interneuron precursors, a cluster of interneurons, a cluster of cerebellar nuclei, and glutamatergic neurons. In further aspects, the cerebellum-like organoids further comprise: choroid plexus or TTR+ cells, meninges or LUM+DCN+ cells, and roof plate cells or GDF7+ cells. The ventricular zone progenitors may be positive for KIRREL2, PTF1A, and VIM. The rhombic lip progenitors may be positive for ATOH1 and BARHL1. The Bergmann glial marker-positive cells may express or be positive for PTPRZ1, EDNRB, GFAP, HOPX, SLCA4A4, and EDNRB. In various aspects, the cluster of neuronal cells comprises the cells expressing the Purkinje cell markers, the Purkinje cell markers comprising SKOR2, RORA, FOXP2, and CALB1; the cluster of interneuron precursor cells express or are positive for PAX2; the cluster of interneurons express or are positive for SOX14 and DMBX1; the cluster of cerebellar nuclei express or are positive for MEIS2, LHX9, and IRX3, and the glutamatergic neurons express or are positive for STMN2 and SLC17A7, wherein the glutamatergic neurons comprise unipolar brush cells (UBC), granule cells (GC), and granule cell progenitors (GCP); or the glutamaterigic neurons comprise a cluster of cells expressing or positive for EOMES, a cluster of cells expressing or positive for NEUROD1 and NHLH1, and a cluster of cells expressing or positive for ATOH1 and BARHL1.

Cerebellum-like organoids are also provided. In various aspects, cerebellum-like organoids are based solely on human cells, wherein the cerebellum-like organoid contains spatially segregated ventricular zone progenitors and rhombic lip progenitors, and the cerebellum-like organoid contains cells expressing Purkinje cell markers. In various aspects, cerebellum-like organoids generated from the methods disclosed above are provided.

In some embodiments, a cerebellum-like organoid is provided, which include spatially segregated ventricular zone progenitors and rhombic lip progenitors; cells expressing Purkinje cell markers; a cluster of Bergmann glial marker-positive cells, a cluster of neuronal cells, a cluster of interneuron precursors, a cluster of interneurons, a cluster of cerebellar nuclei, and glutamatergic neurons; choroid plexus or TTR+ cells, meninges or LUM+DCN+ cells, and roof plate cells or GDF7+ cells. For instance, the ventricular zone progenitors are KIRREL2+, PTF1A+, and VIM+; the rhombic lip progenitors are ATOH1+ and BARHL1+; the Bergmann glial marker-positive cells express or are positive for PTPRZ1, EDNRB, GFAP, HOPX, SLCA4A4, and EDNRB; the cluster of neuronal cells comprises the cells expressing the Purkinje cell markers; the cluster of interneuron precursors express or are positive for PAX2; the cluster of interneurons express or are positive for SOX14 and DMBX1; the cluster of cerebellar nuclei express or are positive for MEIS2, LHX9, and IRX3; and the glutamatergic neurons express or are positive for STMN2+ and SLC17A7+, wherein the glutamatergic neurons comprise unipolar brush cells (UBC), granule cells (GC), and granule cell progenitors (GCP); or the glutamaterigic neurons comprise a cluster of cells expressing or positive for EOMES, a cluster of cells expressing or positive for NEUROD1 and NHLH1, and a cluster of cells expressing or positive for ATOH1 and BARHL1.

Methods method for screening a candidate drug are also provided, which include contacting a cerebellum-like organoid provided herein with a candidate drug, and assaying survival, activity, and/or expression of a neural marker of the cerebellum-like organoid in response to the candidate drug. In some embodiments, the cerebellum-like organoid may include a genetic alteration associated with a neurological disease or condition, so that a candidate drug is assayed with the cerebellum-like organoid containing the genetic alteration.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIGS. 1A-1H. Human cerebellar organoids (hCerOs) reproducibly generate the cellular diversity of the human cerebellum. (1A) Protocol schematic for generating cerebellar organoids; ROCKi, Y-27632-ROCK inhibitor; SB, SB431542-TGFB inhibitor; Noggin, BMP inhibitor; CHIR, CHIR-99021-GSK3 inhibitor; FGF8b, Fibroblast Growth Factors 8b; T3: triiodothyronine; BDNF, brain-derived neurotrophic factor. (1B) Schematic of the developing human cerebellar plate (sagittal view). EGL: external granular layer; NTZ: nuclear transitory zone; SVZ: sub ventricular zone; VZ: ventricular zone. (1C) Immunofluorescence staining of KIRREL2+ ventricular zone progenitors and ATOH1+ rhombic lip progenitors on 1-month-old hCerO. SOX2 defines early neuroepithelium. Scale bar: 200 mm. (1D) Normalized mean reverse-transcription quantitative PCR (RT-qPCR) expression for region-specific markers among various brain region-specific organoids at day 16. (1E) VoxHunt analysis on neuronal subtypes (GCP, GC, eCN/UBC, iCN, PIP, immature PC, PC, and MLI) for PCW 12-24. STR, striatum; NCx, neocortex; HIP, hippocampus; DTH, dorsal thalamus; CB, cerebellum; AMY, amygdala. (1F) Uniform Manifold Approximation and Projection (UMAP) plot of scRNA-seq data from 2-month organoids after canonical correlation analysis (CCA) batch correction and alignment (D2: one batch; n=3 individual organoids combined). Left UMAP: combined organoids, colored by cell types. Right UMAP: human dataset. Ast: astrocyte, BG: Bergmann glia, BS: brainstem, Div-: dividing, eCN/Unibrush: excitatory cerebellar nuclei and unipolar brush cells, GC: granule cells, iCN: inhibitory cerebellar nuclei, ML: molecular layer interneurons, OPC: oligodendrocyte progenitor cells, PIP: pax2+ interneuron progenitors, PC: purkinje cells, RL: rhombic lip, RG: radial glia, VZ: ventricular zone. (1G and 1H) Violin plot of key markers that identify major cell types of the developing CB in organoids vs. human fetal tissue.

FIGS. 2A-2H. Identification of human-specific rhombic lip ventricular and subventricular subsets. (2A) UMAP unbiased clustering and DEG-based annotation of the RL and dividing-VZ subcluster (n=1,629 cells; n=788 for SVZ; n=645 for VZ, n=196 for IZ). IZ, intermediate zone; SVZ, subventricular zone; VZ, ventricular zone. (2B) UMAP visualization of cells grouped by sample (n=611 for hCerO and n=1,018 for fetal) (hCerO vs. fetal). (2C and 2D) UMAP visualization of cells split by sample (hCerO vs. fetal). (2E-2G) Heatmap showing the hCerO cells' expression of human single-cell DEGs used to identify IZ, SVZ, and VZ. (2H) Dot plot showing the expression of selected marker genes in each subcluster.

FIGS. 3A-3O. hCerOs display organized laminar layering reminiscent of the external granule cell layer (EGL) and the Purkinje Cell layer (PCL). (3A) Schematic representation of in vivo human development around Carnegie Stage 23 (CS23) [56 days post conception]; EGZ, external granule zone or EGL. (3B) Feature plot of CXCR4 (receptor for SDF1a) within 2-month-old hCerOs. (3C) Immunofluorescence of hCerOs BARHL1 (granule cell progenitors) and CXCR4 (SDF1a receptor). Scale bars: 200 mm. (3D-3I) Immunofluorescence of hCerOs BARHL1, SKOR2, and PAX6 (excitatory granule cell progenitor marker) (3D-3F) (−SDF1a) and (3G-3I) (+SDF1a) are consecutive sections. Scale bars: 200 mm. (3J-3M) Immunofluorescence of hCerOs BARHL1 (granule cell progenitors) and CALB1 (Purkinje cells). Scale bars: 200 mm. (3N) Binning quantification of BARHL1+ cells. Student's t-test was performed on total of 12 regions for each condition from 3 independent experiments. (3O) 6-month-old hCerO no longer treated with SDF1a. ***p-value<0.001, ****p-value<0.0001. Scale bars: 200 mm.

FIGS. 4A-4R. hCerOs display functionally mature network activity in long-term cultures, resembling patterns of in vivo cerebellar circuits. (4A) Schematic showing functional characterization of 2- and 6-month-old hCerOs. (4B) Representative 2 mo old 11a organoid transduced with SomaGCaMP6f2. Scale bar: 50 mm. (4C) Spontaneous calcium signal traces as AF/F in 2-month-old 11a-derived hCerOs. (4D) Spontaneous calcium signal traces as pseudocolor heatmap. (4E) Representative heatmap as AF/F after bath application of 1 μM TTX of a 2-months-old 11a organoid. (4F) Representative pseudocolor heatmap after bath application of 1 mM TTX of a 2-month-old 11a organoid. (4G) Representative clustering identification on a 2-month-old hCerO. (4H) Quantification of number of clusters in n=48 2-month-old hCerOs. Dots represent average value for each recorded field; bars: standard deviation. (4I) Quantification of average activation for frames for n=14 2-month-old and n=14 6-month-old hCerOs. Student unpaired t test of ****p-value<0.0001. Dots represent max average value for each recorded field; bars: standard deviation. (4J) Quantification of mean interpeak times for n=14 2-month-old and n=14 6-month-old hCerOs. Student unpaired t test **p-value=0.0022. Dots represent average value for each recorded field; bars: standard deviation. (4K) Quantification of correlation coefficient of calcium events in n=14 2-month-old and n=14 6-month-old hCerOs. Student unpaired t test **p-value=0.0020. Dots represent average value for each recorded field; bars: standard deviation. (4L) Quantification of burstiness in each cluster for n=14 2-month-old and n=14 6-month-old hCerOs. Student unpaired t test ***p-value<0.0001. Dots represent average value for each recorded organoid; bars: standard deviation. (4M) Representative heatmap after bath application of 3 μM NMDA of a 2-months-old 11a organoid. (4N) Representative heatmap after bath application of 3 μM NMDA and 3 μM PTX of a 2-months-old 11a organoid. (4O) One way ANOVA comparison of burstiness in each cluster of n=6 2-month-old and n=7 6-month-old hCerOs treated with NMDA and NMDA+PTX. Dots represent average value for each recorded organoid; bars: standard deviation. ***p-value=0.005, **p-value=0.0065. (4P) Schematic representation of 6-month-old hCerO transduction with AAV8-SomaGCaMP6f and lentivirus L7-eOPN3. Optogenetic stimulation was performed with 520 nm LED. Calcium imaging was performed with two-photon microscopy. (4Q) Representative images of maximum projection of stacks acquired during calcium imaging recording. Scale bar: 100 mm. (4R) Analysis of average intensity per frame from calcium imaging analysis in n=3 eOPN3- and n=8 eOPN3+ infected organoids. Each dot represents an organoid. Student paired t test ***p value=0.0001.

FIGS. 5A-5M. Functionally mature, human Purkinje neurons develop within long-term culture of hCerOs. (5A) Schematic representation of spatial transcriptomics conducted on hCerOs. (5B) Differential expression of genes between cells identified as Purkinje and non-Purkinje cells in 6-month-old hCerOs. Significant changes (p-value<0.05) are marked with solid circle. (5C) UMAP feature plots showing cell-level expression of Purkinje cell marker genes identified with overlapping expression of high CA8 levels. (5D) Spatial localization of upregulated Purkinje cell genes within a section of 6-month-old hCerO. (5E) Upper panel: schematic of patch-clamp recording of PCP2+ neurons from intact hCerOs. Lower panel: representative PCP2+ neuron. Imaged with SP8-8Xmicroscope. Scale bar: 200 μm. (5F) Representative whole-cell patch-clamp trace of induced AP from PCP2+ neuron from intact hCeOs. (5G) Representative whole-cell patch-clamp trace of spontaneous firing from PCP2+ neuron from intact hCerOs. (5H) Quantification of peak amplitude for AP elicited from n=14 recorded cell. Mean+SD. Each dot represents a single AP from an individual neuron. (5I) Quantification of AP frequency from 8 spontaneously firing cells. Mean+SD. Each dot represents a recorded neuron. (5J) Quantification of Ih current for n=10 Inward Sodium/Outward Potassium Rectified currents. Mean+SD. Each dot represents a recorded neuron. (5K) Representative whole-cell patch-clamp trace of Ih current from PCP2+ neuron from intact hCeOs. (5L) Representative whole-cell patch-clamp trace of repetitive spontaneous firing from PCP2+ neuron from intact hCerOs. (5M) Drawings of human Purkinje cells at various developmental stages (modified from Zecevic et al., J. Comp. Neur. 1976) compared with 6-month-old hCerO stained for CALB1. Imaged with Leica Model TL LED Thunder widefield scope. Scale bars: 200 μm.

FIGS. 6A-6N. Human cerebellar organoid protocol optimization. (6A-6E) PGPFOXG1 derived organoids expressing various levels of FOXG1 depending on organoid protocol: (6A) Forebrain control (6B) gfCDM+insulin (gfCDM+i) +FGF2 (6C) CerDM1+CHIR+FGF8b (6D) gfCDM+i+CHIR+FGF8b (6E) Day6 switch. (6F) Quantification of levels of FOXG1 red signal in Day 16 organoids of various protocols. Forebrain control: n=18, gfCDM+i+FGF2: n=39, CerDM1+CHIR+FGF8b: n=26, gfCDM+i+CHIR+FGF8b: n=26, Day 6 switch: n=26. (6G) Day 16 qPCR of regional identity markers across various brain region specific organoids normalized to forebrain organoid levels. n=16 qPCR fold-change values for various brain region specific organoids. (6H) Schematic representation of gradual exposure to CerDM1 media after initial seeding in gfCDM+i media. (6I) Quantification on levels of FOXG1 red signal in Day 16 organoids using PGPFOXG1 reporter cell line to derive organoids from FIG. 6H. (6J-6M) Day 16 qPCR of markers identifying forebrain (FOXG1), midbrain/hindbrain boundary (FGF8), hindbrain (GBX2), and midbrain (OTX2). (6N) Quantification of superimposed images of ATOH1 and KIRREL2 stained consecutive sections n=3. *p-value<0.05, **p-value<0.01, ****p-value<0.0001.

FIG. 7. Reproducible generation of cerebellar cells across cell lines. Quantification of percentage of BARHL1 and SKOR2 cells across various cell lines from 3 organoids from 3 different batches. BARHL1: excitatory granule cell progenitors; SKOR2: inhibitory post-mitotic newborn Purkinje cells.

FIGS. 8A-8L. Reproducibility of functional hCerO across cell lines. (8A) 2-month-old organoids from two different cell lines (D2 and PGP1) transduced with SomaGCaMP6f2. Scale bar: 200 m. (8B) Spontaneous calcium signal traces for each example cell as AF/F. Representative of calcium signal per cell line (D2, PGP1). (8C) Spontaneous calcium signal traces as heatmap. Representative of calcium signal per cell line (D2, PGP1). (8D) Quantification of average activation for frames for each 2-month-old hCerO. One way ANOVA comparison of 9 orgs (11a), 9 orgs (D2), 9 orgs (PGP1). Dots represent average value for each recorded organoid; bars: standard deviation. (8E) Quantification of average amplitude for each 2-month-old hCerO. One way ANOVA comparison of 9 orgs (11a), 9 orgs (D2), 9 orgs (PGP1). Dots represent average value for each recorded organoid; bars: standard deviation. (8F) Quantification of average event length for each 2-month-old hCerO. One way ANOVA comparison of 9 orgs (11a), 9 orgs (D2), 9 orgs (PGP1). Dots represent average value for each recorded organoid; bars: standard deviation. (8G) Analysis of number (%) of active ROIs in baseline and TTX condition of 6 2-months-old hCerO. Each dot represents an organoid. Student unpaired t test **p-value=0.0001. (8H) One way ANOVA comparison of average intensity per frame of n=7 2-months-old hCerO in baseline, NMDA, NMDA+AP5, and NMDA+AP5+PTX. Each dot represents an organoid. **p-value=0.0001. (8I) Schematic representation of 6-months-old hCerO transduction with AAV8-SomaGCaMP6f only without Lentivirus L7-eOPN3. Optogenetic stimulation was performed with 520 nm LED. Calcium imaging was performed with 2-photon microscopy.

FIG. 9 is a schematic depicting the preparation and exemplary analysis of human cerebellar organoids.

FIGS. 10A-10B. Top 100 Purkinje Cell DEGs from human fetal dataset (10B) compared to 2-month-old hCerO (10A).

FIGS. 11A-11F. hCerO cell type enrichment in various diseases. (11A-11F) Heatmaps of mean expression per organoid cerebellar cell type for genes associated with various diseases (11A, intellectual disability; 11B, autism spectrum disorder; 11C, spinocerebellar ataxia; 11D, Joubert syndrome; 11E, Alzheimer's disease; 11F, cerebellar malformations). Color scheme is based on z-score distribution. In the heatmaps, each row represents one gene, and each column represents a single cell type. Gene expression was clustered by row. Enrichment p-values (−log 10P value) for each cell type are shown in the bottom bar plots. Significance was determined by one-sample z-test, two-tailed p-value. The horizontal line in the bar plots represents the 0.05 significance threshold.

FIGS. 12A-12F. GCaMP parameters across cell lines and between organoids. (12A-12C) 2-month-old organoids from PGP (12A), 11a (12B), and D2 (12C) cell lines. Each dot represents a recorded field. (12D-12F) 6-month-old organoids from PGP (12D), 11a (12E), D2 (12F) cell lines. Each dot represents a recorded field.

FIGS. 13A-13C. Spatial Transcriptomics of 6-month-old organoids. (13A) UMAP of significantly enriched genes in the Purkinje cell cluster of 6-month-old hCerOs. n=2 organoids. (13B) Spatial localization of significantly enriched genes in PCs in one 10 uM section of 6-month-old organoid. n=2 organoids. (13C) Spontaneous action potential without (left panel) and with luM TTX (right panel).

FIG. 14. hCerO differentiation timeline. (panel a) Timeline of Day 0 dissociation and platting of stem cells to initiate hCerO differentiation. (panel b) Timeline of cerebellar induction and patterning protocol for generating hCerOs along with key developmental stages, cellular identity, and culture conditions. NE: neuroepithelium, PC: Purkinje cell, GC: granule cell, PIP: PAX2+ interneuron progenitors, UBC: unipolar brush Cells, iCN/eCN: inhibitory and excitatory cerebellar nuclei, ROCKi: Y-27632-ROCK inhibitor, SB: SB431542-TGFB inhibitor, Noggin: BMP inhibitor, CHIR: CHIR99021-GSK3 inhibitor, FGF8b: fibroblast growth factor 8b, gfCDM+i: growth factor-free chemically defined media+insulin, CerDM1/2/3: cerebellar differentiation media 1/2/3, T3: triiodothyronine, BDNF: bone derived neurotrophic factor, SDF1a: stromal cell-derived factor 1a.

FIG. 15. Morphological features of early hCerOs up to Day 16. (panel A) Brightfield images of cerebellar organoids of various pluripotent stem cell lines. (panel B) Day 16 organoids from 3 different cell lines. (panel C) Brightfield images of cerebellar organoids derived from PGP1 cell line comparing the original protocol (switch to CerDM1 on day 6 up to day 16) vs remaining in gfCDM+i all the way through the patterning phase of the differentiation (day 0-16).

FIG. 16. Morphological and immunohistochemical features of Day 30+hCerOs. (panel A) Brightfield image of hCerO (gfCDM+i from d0-6, CerDM1 from d6-16) at Day 30. Scale bar: 200 um. (panel B) Immunostaining of key cerebellar markers including BARHL1 (granule cell progenitors) and SKOR2 (post-mitotic Purkinje Cell progenitor) and their respective progenitor pools (ATOH1 (rhombic lip progenitor cells) and KIRREL2 (ventricular zone progenitor cells)). SOX2 stains neuroepithelial progenitors. (panel C) Brightfield image of modified hCerO (gfCDM+i from day 0-16) at Day 30. Scale bar: 200 um. (panel D) Immunostaining of key cerebellar markers including BARHL1 (granule cell progenitors) and SKOR2 (post-mitotic Purkinje Cell progenitor) and their respective progenitor pools (ATOH1 (rhombic lip progenitor cells) and KIRREL2 (ventricular zone progenitor cells)). SOX2 stains neuroepithelial progenitors and endogenous tdTOMATO signal from FOXG1 reporter cell line. (panel E) Brightfield image of day 60 organoids. Arrows indicate the presence of choroid plexus-like cells. (panel F) 2-month old hCerOs stained with CALB1. Arrows indicate the presence of dendrites. Scale bar: 50 um. (panel G) 6-month old hCerOs stained with CALB1. Scale bar: 50 um.

FIG. 17. Morphological and immunohistochemical features of suboptimal outcomes. (panel A) Brightfield image of an unsuccessful seeding with significant cell death surrounding the central ball in a V-bottom plate. (panel B) Brightfield image of an improperly patterned organoid leading to a forebrain identity with many small rosettes within the organoid. (panel C) Lower magnification of unsuccessful pattered organoid batch leading to some organoid with forebrain-like identity indicated by black arrows. (panel D) Brightfield image of day 60 organoids with a high amount of choroid plexus-like cells taking over the organoid. (panels E-G) Immunofluorescence images of 20 um thick, consecutive sections of day 60 organoid stained with BARHL1, PAX6, MAP2, SOX2, TBR1, and endogenous tdTOMATO signal deriving from a FOXG1 reporter cell line. Scale bar, 200 um.

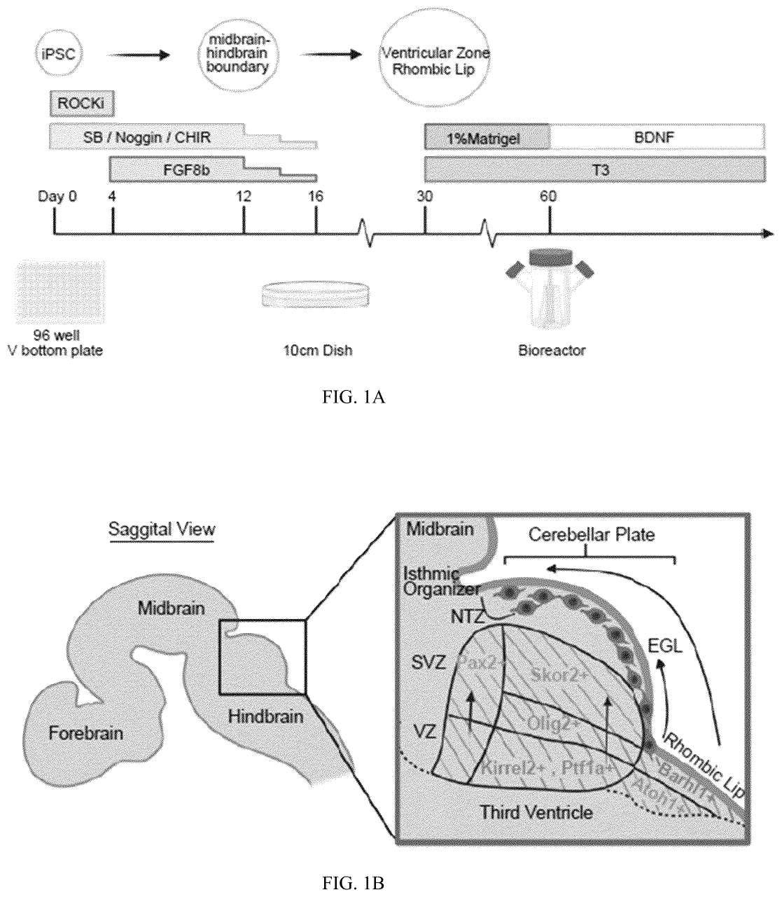

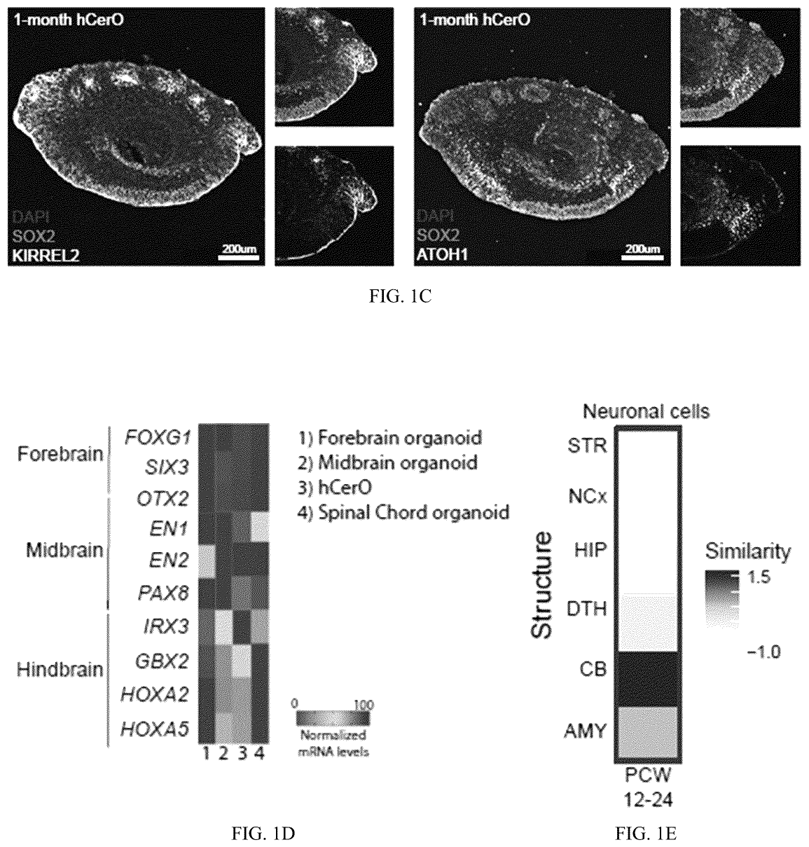

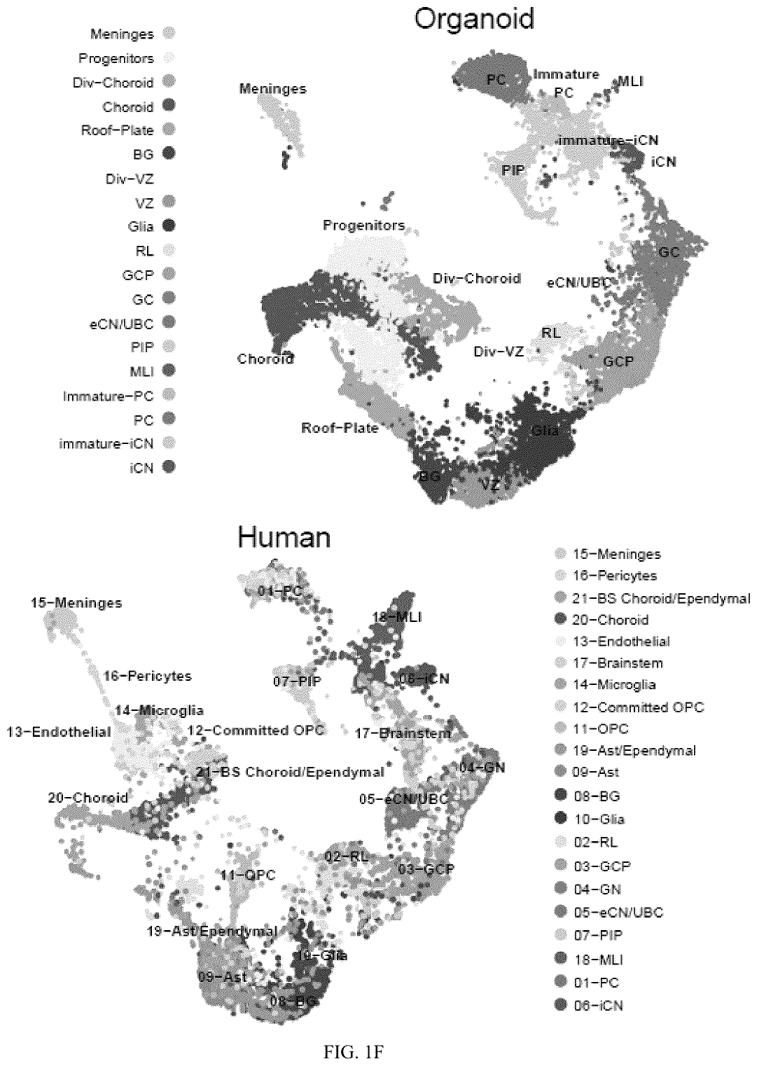

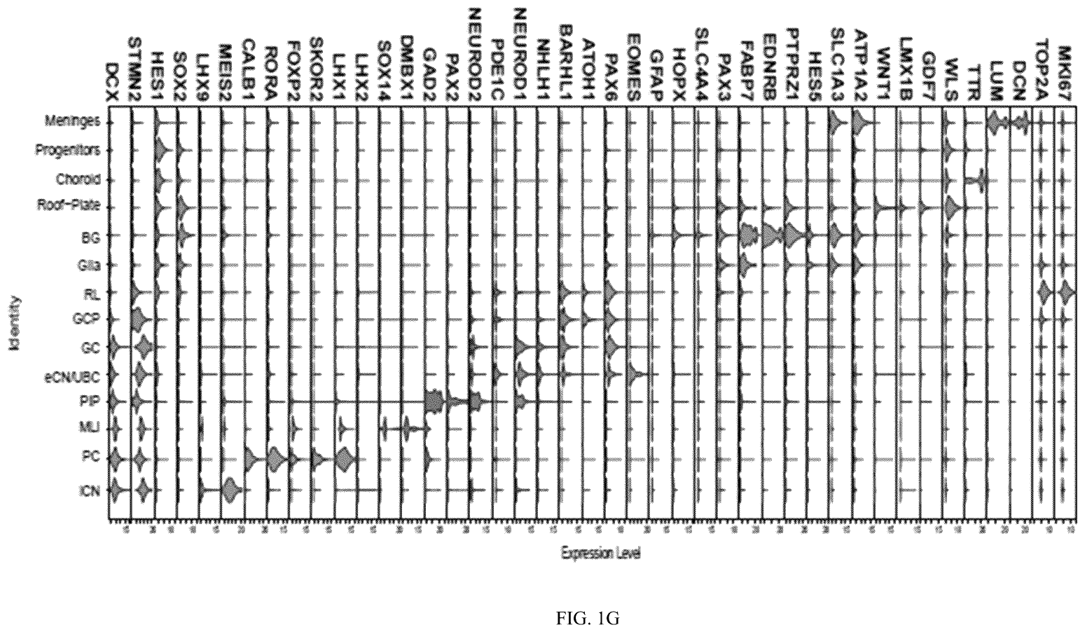

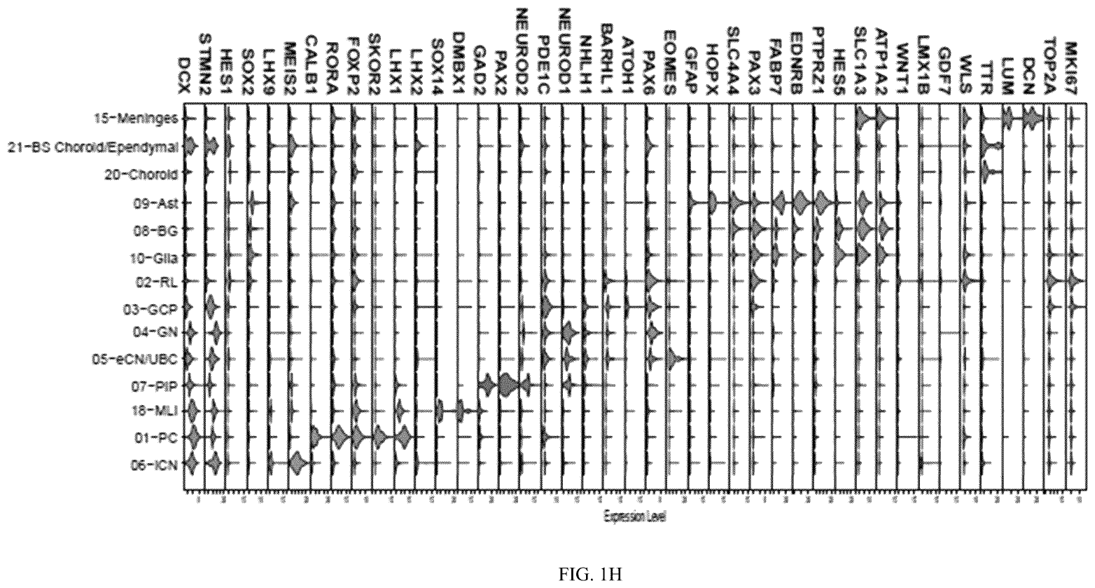

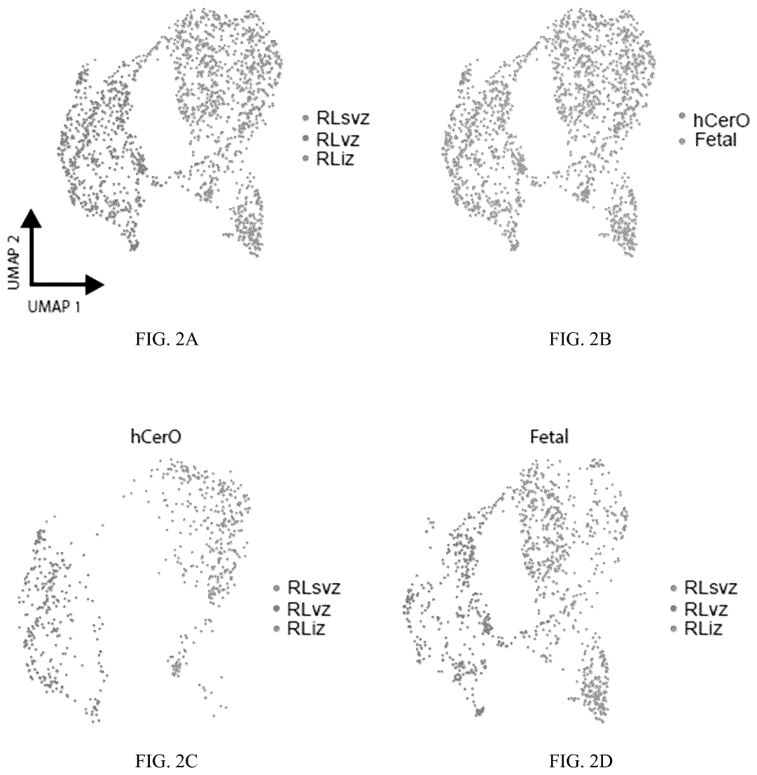

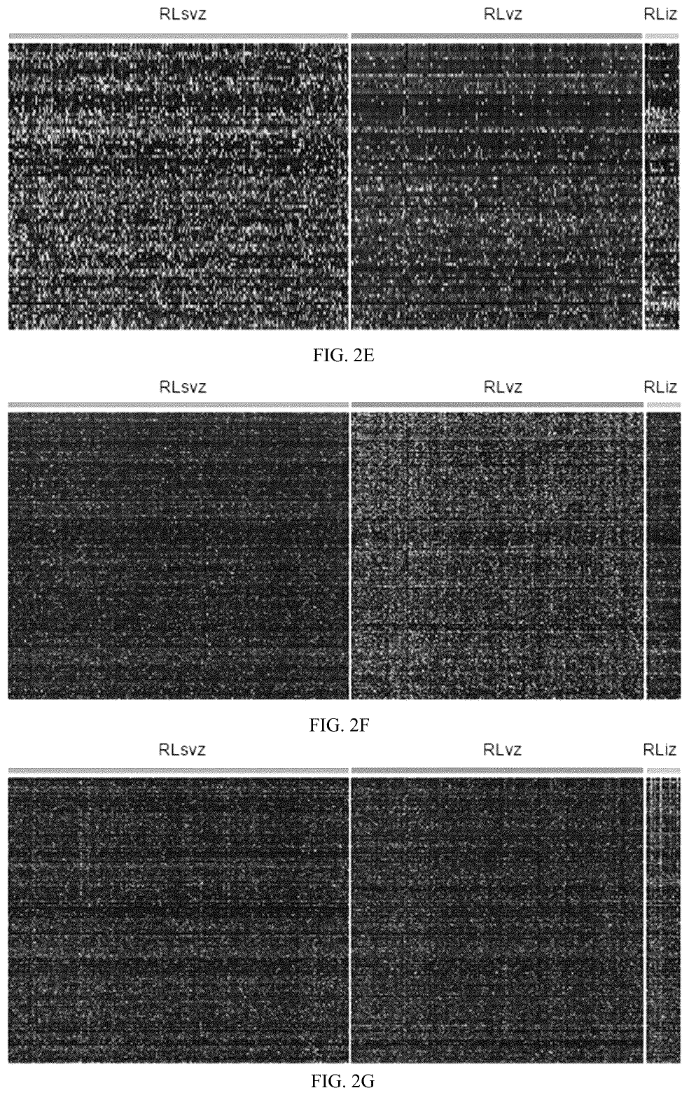

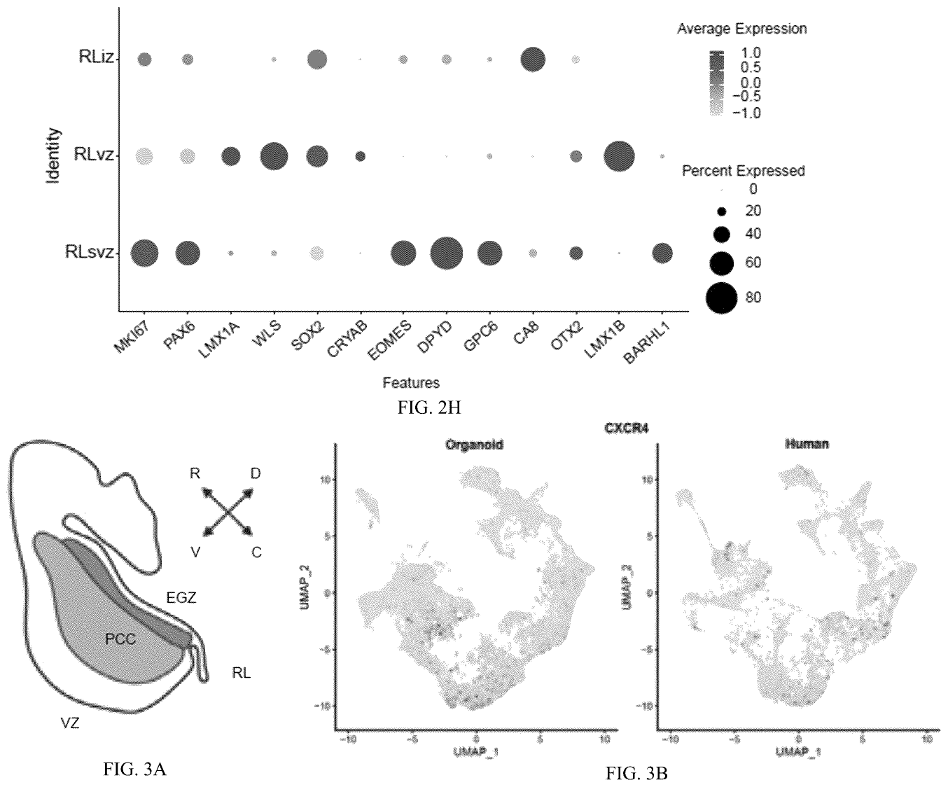

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. Sep; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “organoid” refers to a miniaturized and, in some cases, a simplified version of an organ produced in vitro in three dimensions, which show realistic and anatomically correct micro-anatomy. They are derived from one or more cells from a tissue, embryonic stem cells or induced pluripotent stem cells, which are capable of self-organization in three-dimensional culture owing to, for example, their self-renewal and differentiation capacities. In various embodiments, an organoid of the invention is derived from human embryonic stem cells or human induced pluripotent stem cells.

The term “embryoid bodies” refers to three-dimensional aggregates of pluripotent stem cells. The pluripotent cell types that comprise embryoid bodies include, but are not limited to, embryonic stem cells (ESCs) derived from the blastocyst stage of embryos from, preferably human (hESC) source. Additionally, embryoid bodies can be formed from pluripotent stem cells derived through alternative techniques, including somatic cell nuclear transfer or the reprogramming of somatic cells to yield induced pluripotent stem cells (iPS).

The term “neuronal lineage embryoid bodies” refers to embryoid bodies wherein the pluripotent stem cells have the potential to generate or become neurons.

The term “midbrain”, also known as the mesencephalon, refers to a section of the brain that is located within the brainstem, below the cerebral cortex, and above the hindbrain.

The term “midbrain-hindbrain regionalized tissue” refers to tissue specific to the midbrain-hindbrain region.

The term “stem cell” refers to undifferentiated biological cells that are capable of differentiating into more specialized cells and that are capable of dividing (through mitosis) to produce more stem cells. Stem cells are found in multicellular organisms. In mammals, there are two broad types of stem cells: embryonic stem cells, which are isolated, for example, from the inner cell mass of blastocysts, and adult stem cells, which are found in various tissues. In adult organisms, stem cells and progenitor cells function as a repair system for the body by replenishing adult tissues. In a developing embryo, stem cells can differentiate into all the specialized cells—derived from any one of the three primary germ layers, called ectoderm, endoderm and mesoderm, present in the early stages of embryonic development—but also maintain the normal turnover of regenerative organs, such as blood, skin, or intestinal tissues.

The three commonly known, accessible sources of autologous adult stem cells in humans are the bone marrow, which requires extraction by harvesting cells, usually from the femur or iliac crest; adipose tissue (lipid cells), which requires extraction by liposuction, and blood, which requires extraction, usually through an apheresis machine. Stem cells can also be taken from umbilical cord blood just after birth. Of all stem cell types, autologous harvesting involves the least risk. By definition, autologous cells are obtained from one's own body. Adult stem cells are frequently used in various medical therapies (e.g., bone marrow transplantation). It is now possible to artificially grow and transform (differentiate) stem cells into specialized cell types, with characteristics consistent with cells of various tissues such as muscles or nerves.

Any mammalian stem cell, preferably human stem cell, can be used in accordance with the methods of the invention as disclosed herein, including but not limited to, stem cells isolated from cord blood, placenta and other sources. In various embodiments, the stem cells are obtained from a human. The stem cells may include pluripotent cells, which are cells that have complete differentiation versatility, that are self-renewing, and can remain dormant or quiescent within tissue. The stem cells may also include multipotent cells or committed progenitor cells. In one example, the method as disclosed herein is performed without the use of human embryonic stem cells; and instead of human embryonic stem cells, other types of pluripotent cells can be used in accordance with the present invention. In another example, the method as disclosed herein is performed on induced pluripotent stem cells, preferably of human origin.

A cell culture media used in cell culture usually comprises at least the following items: a carbon or carbohydrate source (for example glucose or glutamine) as a source of energy; amino acids for protein synthesis; vitamins, which promote cell survival and growth; a balanced salt solution, usually a mixture of various ions to maintain optimal osmotic pressure within the cells and to act as cofactors for various cofactor-mediated reactions (for example cell adhesion, enzymatic reactions and the like); a pH indicator (for example phenol red; indicating a change in pH from neutral to basic or acidic, which usually indicate the presence of nutrient depletion, contamination, accumulation of necrotic cells and the like) and a buffer (for example, bicarbonate or HEPES buffer) to maintain the required pH in the media. In addition to the components listed above, cell culture media can be modified. For example, the use of fetal calf or bovine serum is required for the growth and maintenance of some cell lines in vitro, but not required for some and to be avoided in others, for example when serum-starved cells are required for cytokine analysis. As defined in the art, a “defined medium” (also known as “chemically defined medium” or “synthetic medium”) is a cell culture medium in which all the chemicals used are known and no yeast, animal, or plant tissue is present.

The term “inhibitor” refers to a molecule or compound that is capable of decreasing, downregulating or, in some cases, completely ceasing, activity of a target molecule. An inhibitor is usually characterized and named for its target; for example, a compound that binds to an enzyme and thereby decreases its activity is called an enzyme inhibitor. An inhibitor can be either reversible or irreversible, meaning that in terms of binding to its target, this target binding may be subsequently broken (reversible) or not (irreversible). Inhibition of a target molecule can, for example, be competitive, uncompetitive, non-competitive, or mixed. Conversely, a molecule or compound that is capable of increasing, upregulating or initiating the activity of a target molecule is known as an activator.

The term “TGF-β inhibitor” refers to a molecule or compound that is capable of blocking or down regulating the effect of TGF-β. TGF-β is a polypeptide member of the transforming growth factor beta superfamily of cytokines. It is a secreted protein that performs many cellular functions, including, but not limited to, the control of cell growth, cell proliferation, cell differentiation and apoptosis. In humans, TGF-β1 is encoded by the TGFB1 gene. Other members of this superfamily include, but are not limited to, bone morphogenetic proteins (BMPs), growth and differentiation factors (GDFs), anti-mullerian hormone (AMH), Activin (for example, Activin A, B and AB), Nodal and different TGF-β's (for example, TGFβ-1, TGFβ-2, TGFβ-3). For example, the TGF-β inhibitor is, but is not limited to, A83-01 3-(6-Methyl-2-pyridinyl)-N-phenyl-4-(4-quinolinyl)-1H-pyrazole-1-carbothioamide, A 77-01 4-(3-(6-methylpyridin-2-yl)-1H-pyrazol-4-yl)quinoline, SD-208 2-(5-chloro-2-fluorophenyl)-N-pyridin-4-ylpteridin-4-amine, LY2157299 4-[2-(6-methylpyridin-2-yl)-5,6-dihydro-4H-pyrrolo[1,2-b]pyrazol-3-yl]quinoline-6-carboxamide, SB 431542 4-[4-(1,3-benzodioxol-5-yl)-5-pyridin-2-yl-1H-imidazol-2-yl]benzamide, GW788388 N-(oxan-4-yl)-4-[4-(5-pyridin-2-yl-1H-pyrazol-4-yl)pyridin-2-yl]benzamide, SB505124 2-[4-(1,3-benzodioxol-5-yl)-2-tert-butyl-1H-imidazol-5-yl]-6-methylpyridine, SB525334 6-[2-tert-butyl-5-(6-methylpyridin-2-yl)-1H-imidazol-4-yl]quinoxaline, IN 1130 2-[3-[(4-amino-2-methylpyrimidin-5-yl)methyl]-4-methyl-1,3-thiazol-3-ium-5-yl]ethanol, ITD 1 (6,6-dimethyl-5,7-dihydroimidazo[2,1-b][1,3]thiazol-4-ium-3-yl)methyl N,N′-dicyclohexylcarbamimidothioate, LY2109761 4-[2-[4-(2-pyridin-2-yl-5,6-dihydro-4H-pyrrolo[1,2-b]pyrazol-3-yl)quinolin-7-yl]oxyethyl]morpholine, K02288 3-[6-amino-5-(3,4,5-trimethoxyphenyl)pyridin-3-yl]phenol, TGF-1 RI kinase inhibitor [3-(Pyridin-2-yl)-4-(4-quinonyl)]-1H-pyrazole] or derivatives thereof.

The term “SMAD inhibitor” refers to a compound of molecule capable of inhibiting (that is preventing or downregulating) the activity of a SMAD protein. The term “SMAD” refers to intracellular proteins that transduce extracellular signals from transforming growth factor beta (TGF-β) ligands to the nucleus where they activate downstream gene transcription. The SMADs, which form a trimer of two receptor-regulated SMADs and one co-SMAD, act as transcription factors that regulate the expression of certain genes. Other SMAD proteins are, but are not limited to, SMAD1, SMAD2, SMAD3, SMAD4, SMAD5, SMAD6, SMAD7, and SMAD8/9.

The term “neurotrophic factor” refers to a family of biomolecules—nearly all of which are either peptides or small proteins—that support the growth, survival, and differentiation of both developing and mature neurons. Most neurotrophic factors exert their trophic effects on neurons by signaling through tyrosine kinases, usually a receptor tyrosine kinase. In the mature nervous system, they promote neuronal survival, induce synaptic plasticity, and modulate the formation of long-term memories. Neurotrophic factors also promote the initial growth and development of neurons in the central nervous system and peripheral nervous system and that they are capable of re-growing damaged neurons in test tubes and animal models. Exemplary neurotrophic factors include, but are not limited to, neurotrophin, glial cell-line derived neurotrophic factor family ligand (GFL), and neuropoietic cytokine. Exemplary neurotrophin is, but is not limited to, nerve growth factor (NGF), brain-derived neurotrophic factor (BDNF), neurotrophin-3 (NT-3) and neurotrophin-4 (NT-4).

The term “growth factor” is used generally consistent with the meaning in the art. It describes a naturally occurring substance capable of stimulating cellular growth, proliferation, healing, and/or cellular differentiation. In various embodiments, a growth factor is a protein. In some embodiments, a growth factor includes a protein hormone.

Animal extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the extracellular matrix (ECM). Basement membranes are sheet-like depositions of extracellular matrix, on which various epithelial cells rest. Each type of connective tissue in animals has a type of extracellular matrix: collagen fibres and bone mineral comprise the extracellular matrix of bone tissue; reticular fibres and ground substance comprise the extracellular matrix of loose connective tissue; and blood plasma is the extracellular matrix of blood. The extracellular matrix is composed of an interlocking mesh of fibrous proteins and glycosaminoglycans (GAGs). Thus, an extracellular matrix can comprise, but is not limited to, proteoglycans (for example, heparan sulfate, chrondroitin sulfate, keratin sulfate), non-proteoglycan polysaccharides (for example, hyaluronic acid), proteins (for example, collagen, elastin) and other components, such as, but not limited to fibronectin and laminin.

The term “genetic alteration” includes a mutated gene and/or an affected expression or function of a gene product. In some embodiments, a genetic alteration is a mutated gene, i.e., a gene whose sequence has been modified by transitions, transversions, deletions, insertions, or other modifications. In some aspects, a normal gene has allelic variants that are not associated with disease and are considered to be a wild-type version of the gene, and hence a mutant gene is relative to a normal gene in terms of effecting a different or lack of function of the gene product, thereby being associated with a disease.

The term “neural marker” refers to any protein or polynucleotide the expression of which is associated with a neural cell fate. Exemplary neural markers include markers associated with the cortex, retina, cerebellum, brain stem, granular neurons, dopaminergic, and GABAergic neurons. Exemplary cerebellar markers include but are not limited to ATOH1, PAX6, SOX2, LHX2, and GRID2. Exemplary markers of dopaminergic neurons include but are not limited to tyrosine hydroxylase, vesicular monoamine transporter 2 (VMAT2), dopamine active transporter (DAT) and Dopamine receptor D2 (D2R). Exemplary cortical markers include, but are not limited to, doublecortin, NeuN, FOXP2, CNTN4, and TBR1. Exemplary retinal markers include but are not limited to retina specific Guanylate Cyclases (GUY2D, GUY2F), Retina And Anterior Neural Fold Homeobox (RAX), and retina specific Amine Oxidase, Copper Containing 2 (RAX). Exemplary granular neuron markers include, but are not limited to SOX2, NeuroD1, DCX, EMX2, FOXG1, and PROX1. Exemplary brain stem markers include, but are not limited to FGF8, INSM1, GATA2, ASCL1, GATA3. Exemplary spinal cord markers include, but are not limited to, homeobox genes including but not limited to HOXA1, A2, A3, B4, A5, C8, or D13. Exemplary GABAergic markers include, but are not limited to, NKCC1 or KCC2. Exemplary astrocytic markers include, but are not limited to, GFAP. Exemplary oliogodendrocytic markers include, but are not limited to, OLIG2 or MBP. Exemplary microglia markers include, but are not limited to, AIF1 or CD4. Exemplary vascular markers include, but are not limited to, NOS3.

The midbrain-hindbrain boundary (NMB), also called the isthmic organizer, is located at the interface of the midbrain and hindbrain neuromeres, characterized by a constriction in the developing neural tube, and believed to function as a signaling center responsible for patterning cell fates anteriorly in the midbrain and posteriorly in the cerebellum.

Research on human cerebellar development and disease has been hampered by the need for a human cell-based system that recapitulates the human cerebellum's cellular diversity and functional features. Here, we report a human organoid model (hCerOs) capable of developing the complex cellular diversity of the fetal cerebellum, including human-specific rhombic lip progenitor populations and molecular layer interneurons that have never been generated in vitro prior to this study. Importantly, we show, for the first time, the generation of cerebellar neurons that express in vivo canonical markers of Purkinje cells in an all-human system, addressing a long-standing challenge in the field. Over time, hCerOs can form distinct cytoarchitectural features, including laminar organized layering. In addition, they create functional connections between inhibitory and excitatory neurons that display coordinated network activity. Finally, long-term culture of hCerOs allows for healthy survival and maturation of Purkinje cells that display molecular and electrophysiological hallmarks of their in vivo counterparts. Together, our results demonstrate the existence of an intrinsic cellular program that regulates the acquisition of the molecular identity and functional characteristics of the diverse repertoire of human cerebellar neurons. This study therefore provides a physiologically relevant, all-human model system to elucidate the cell type specific mechanisms governing cerebellar development and disease.

Here we report an organoid model that allows for the first time healthy long-term survival and maturation of functional cerebellar cells, including Purkinje neurons, in an all-human 3D context. We show that cerebellar organoids reliably generate spatially segregated ventricular and rhombic lip progenitor zones, which give rise to the cellular diversity of the human cerebellum within and across multiple cell lines. At 2 months in culture, we observed the emergence of an organized laminar layering with inhibitory and excitatory cerebellar neuronal subtypes, which are functionally connected and display coordinated activity, which increases over a period of six months in culture. Finally, long-term culture of cerebellar organoids allows the maturation of Purkinje cells that display the transcriptomic profile and electrophysiological features of functional neurons.

In various embodiments, the method includes culturing the human stem cells and/or stem cell-derived cells (e.g., embryoid bodies derived from the stem cells, tissues or organoids derived from the embryoid bodies) in the presence of two or more SMAD signaling inhibitors. In some embodiments, a SMAD signaling inhibitor is a TGF-β inhibitor. In some embodiments, the two or more SMAD signaling inhibitors are selected from 4-[4-(1,3-benzodioxol-5-yl)-5-pyridin-2-yl-1H-imidazol-2-yl]benzamide (SB 431542), noggin, and derivatives thereof. In some embodiments, the two or more SMAD signaling inhibitors are SB431542 and noggin.

In some embodiments, the TGF-β inhibitor of the two or more SMAD signaling inhibitors is provided, in a cell culture medium, at a concentration of between about 0.5 nM to about 100 μM, or between about 1 nM to about 90 μM, or between about 2 nM to about 80 PM, or between about 3 nM to about 70 μM, or between about 4 nM to about 60 μM, or between about 5 nM to about 50 μM, or between about 10 nM to about 40 μM, or between about 20 nM to about 30 μM, or between about 30 nM to about 20 μM, or between about 40 nM to about 10 μM, or between about 50 nM to about 5 μM, or between about 100 nM to about 2 μM, or between about 200 nM to about 1 μM, or between about 300 nM to about 800 nM, or between about 500 nM to about 700 nM, or about 1 nM, 5 nM, 10 nM, 15 nM, 20 nM, 25 nM, 30 nM, 35 nM, 40 nM, 45 nM, 50 nM, 75 nM, 100 nM, 150 nM, 200 nM, 250 nM, 300 nM, 400 nM, 500 nM, 600 nM, 700 nM, 800 nM, 900 nM, or about 1 μM, 2 μM, 3 μM, 4 μM, 5 μM, 7.5 μM, 10 μM, 12.5 μM, 15 μM, 17.5 μM or 20 μM. In some embodiments, the TGF-β inhibitor is provided in a cell culture medium at a concentration of about 10 μM.

In some embodiments, noggin is provided, in a cell culture medium, at a concentration of between about 5 ng/mL to about 5 mg/mL, or between about 5 ng/mL to about 1 mg/mL, or between about 10 ng/mL to about 800 μg/mL, or between about 20 ng/mL to about 600 μg/mL, or between about 40 ng/mL to about 400 μg/mL, or between about 50 ng/mL to about 200 μg/mL, or between about 100 ng/mL to about 180 μg/mL, or between about 150 ng/mL to about 160 μg/mL, or between about 300 ng/mL to about 150 μg/mL, or between about 500 ng/mL to about 100 μg/mL, or between about 1 μg/mL to about 50 μg/mL, or about 1 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 75 ng/mL, 100 ng/mL, 150 ng/mL, 200 ng/mL, 250 ng/mL, 300 ng/mL, 400 ng/mL, 500 ng/mL, 600 ng/mL, 700 ng/mL, 800 ng/mL, 900 ng/mL, or about 10 μg/mL, 20 μg/mL, 30 μg/mL, 40 μg/mL, 50 μg/mL, 75 μg/mL, 100 μg/mL, 125 μg/mL, 150 μg/mL, 175 μg/mL or 200 μg/mL. In some embodiments, noggin is provided in a cell culture medium at a concentration of about 100 ng/mL or 150 ng/mL.

In some embodiments, the method includes culturing the human stem cells and/or stem cell-derived cells (e.g., embryoid bodies derived from the stem cells, organoids derived from the embryoid bodies) in the presence of a WNT-signaling activator. In some embodiments, a WNT-signaling activator is a GSK3 inhibitor. In some embodiments, the GSK3 inhibitor is CHIR-99021, whose IUPAC name is 6-[2-[[4-(2,4-dichlorophenyl)-5-(5-methyl-1H-imidazol-2-yl)pyrimidin-2-yl]amino]ethylamino]pyridine-3-carbonitrile, or a derivative thereof. Other exemplary GSK3 inhibitors include, but are not limited to, BIO(6-bromoindirubin-3′-oxime), SB 216763 (3-(2,4-dichlorophenyl)-4-(1-methylindol-3-yl)pyrrole-2,5-dione), CHIR-98014 (6-N-[2-[[4-(2,4-dichlorophenyl)-5-imidazol-1-ylpyrimidin-2-yl]amino]ethyl]-3-nitropyridine-2,6-diamine), TWS119 (3-[[6-(3-aminophenyl)-7H-pyrrolo[2,3-d]pyrimidin-4-yl]oxy]phenol), IM-12 (3-[2-(4-fluorophenyl)ethylamino]-1-methyl-4-(2-methyl-1H-indol-3-yl)pyrrole-2,5-dione), 1-azakenpaullone 9-bromo-7,12-dihydropyrido[3′,2′:2,3]azepino[4,5-b]indol-6(5H)-one, AR-A014418 1-[(4-methoxyphenyl)methyl]-3-(5-nitro-1,3-thiazol-2-yl)urea, SB415286 3-(3-chloro-4-hydroxyanilino)-4-(2-nitrophenyl)pyrrole-2,5-dione, AZD1080 (3E)-3-[5-(morpholin-4-ylmethyl)-1H-pyridin-2-ylidene]-2-oxo-1H-indole-5-carbonitrile, AZD2858 3-amino-6-[4-(4-methylpiperazin-1-yl) sulfonylphenyl]-N-pyridin-3-ylpyrazine-2-carboxamide, indirubin (3E)-3-(3-oxo-1H-indol-2-ylidene)-1H-indol-2-one or derivatives thereof.

In some embodiments, the WNT-signaling activator, or the GSK3 inhibitor is provided, in a cell culture medium, at a concentration of between about 0.5 nM to about 100 μM, or between about 1 nM to about 90 μM, or between about 5 nM to about 80 μM, or between about 10 nM to about 70 μM, or between about 25 nM to about 60 μM, or between about 50 nM to about 50 μM, or between about 100 nM to about 40 μM, or between about 150 nM to about 30 μM, or between about 200 nM to about 20 μM, or between about 300 nM to about 10 μM, or between about 500 nM to about 5 μM, or between about 1 μM to about 2 μM, or between about 1.5 μM to about 2 μM, or between about 1.6 μM to about 1.8 μM, or about 1.0, 1.1, 1.2, 1.3, 1.4, 1.5, 1.6, 1.7, 1.8, 1.9, or 2.0 μM.

In some embodiments, the method includes culturing the human stem cells in a medium free of ROCK inhibitors. Removing ROCK inhibitors from a culture medium promotes stem cell proliferation and aggregate growth. Exemplary rho-kinase inhibitors (ROCK inhibitor) include Y-27632 (also referred to as Y27632), Y-30141, Y-33075, Y-39983, AT-13148, BA-210, beta-elemene, belumosudil, Blebbistatin, chroman 1, DJ4, Fasudil, GSK-576371, GSK429286A, H-1152, hydroxyfasudil, ibuprofen, LX-7101, netarsudil, RKI-1447, ripasudil, TCS-7001, thiazovivin, and verosudil. In some embodiments, the ROCK inhibitor is Y27632, which is (1R,4r)-4-((R)-1-aminoethyl)-N-(pyridin-4-yl)cyclohexanecarboxamide, or a derivative thereof.

In some embodiments, the ROCK inhibitor is provided, in a cell culture medium, at a concentration of at a concentration of between about 0.5 μM to about 100 μM, or between about 1 μM to about 80 μM, or between about 5 μM to about 60 μM, or between about 10 μM to about 40 μM, or between about 15 μM to about 30 μM, or about 15, 16, 17, 18, 19, 20, 21, 22, 23, 24, 25, 26, 27, 28, 29, or 30 μM.

In various embodiments, the method includes culturing embryoid bodies derived from human stem cells, or tissues derived therefrom, in the presence of a midbrain-hindbrain morphogen. In some embodiments, the midbrain-hindbrain morphogen is a fibroblast growth factor. In some embodiments, the midbrain-hindbrain morphogen is FGF8, which regulates the size and positioning of the functional areas of the cerebral cortex. In some embodiments, the midbrain-hindbrain morphogen is FGF8b. In other embodiments, other FGFs such as FGF1, FGF2, FGF3, FGF4, FGF5, FGF6, FGF7, FGF9, FGF10, FGF11, FGF12, FGF13, FGF14, and combinations thereof may be used.

In some embodiments, the fibroblast growth factor is at a concentration of between about 0.5 nM to about 100 μM, or between about 1 nM to about 90 PM, or between about 5 nM to about 80 μM, or between about 10 nM to about 60 μM, or between about 20 nM to about 40 PM, or between about 40 nM to about 20 μM, or between about 60 nM to about 10 μM, or between about 80 nM to about 1 μM, or between about 100 nM to about 500 nM, or between about 100 nM to about 300 nM, or about 75, 100, 150, 200, 250, 300, 350, 400, 450, or 500 nM.

In various embodiments, the present invention includes a culture medium comprising a basal growth cell medium and one or more supplements.

In some embodiments, a basal growth cell medium is, but is not limited to Iscove's modified Dulbecco's medium (IMDM), Eagle's minimal essential medium (EMEM), alpha minimum essential medium (aMEM), Dulbecco's modified Eagle's medium (DMEM), Dulbecco's Modified Eagle medium/Nutrient Mixture F-12 (DMEM/F-12), Roswell Park Memorial Institute medium (RPMI or RPMI 1640), Glasgow's Minimal Essential Medium (GMEM), Biggers, Gwatkin, and Judah medium (BGJ), Biggers, Gwatkin, and Judah medium Fitton-Jackson modification (BGJb), Basal Medium Eagle (BME), Brinster's medium for ovum culture (BMOC-3), Connaught Medical Research Laboratories medium (CMRL), neurobasal medium, C02-Independent medium, Ham's F-10 Nutrient Mixture, Ham's F-12 Nutrient Mixture, Improved MEM, medium 199, Leibovitz's L-15, McCoy's 5A, MCDB 131, Media 199, mTeSR media, Minimum Essential Media (MEM), Modified Eagle Medium (MEM), Waymouth's MB 752/1, Williams' Media E, or combinations, known substitutions or modifications thereof.

In some embodiments, the basal cell growth medium is Iscove's modified Dulbecco's medium (IMDM) with Ham's F-12 Nutrient Mixture (IMEM/F12). Specifically, Iscove's Modified Dulbecco's Medium (IMDM) is a medium suited for rapidly proliferating, high-density cell cultures. IMDM is a modification of Dulbecco's Modified Eagle Medium, and IMDM includes selenium as well as additional amino acids and vitamins but lacks iron (with potassium nitrate replacing ferric nitrate) and contains no proteins, lipids, or growth factors.

Ham's F-12 nutrient mix is a medium containing no proteins or growth factors. Compared to other basal media, F-12 contains a wider variety of components, including zinc, putrescine, hypoxanthine, and thymidine.

In some embodiments, the basal cell growth medium is Dulbecco's Modified Eagle's Medium with Nutrient F-12 (DMEM/F12).

In some embodiments, the one or more supplements include N-2 Supplement, which is used for expansion of undifferentiated cells.

In some embodiments, the one or more supplements include B27, which may be used for the maintenance of neurons. In some embodiments, the B27 is without vitamin A. In other embodiments, the one or more supplements includes B27 and vitamin A.

In some embodiments, the one or more supplements include heparin, which may be used for cell proliferation.

In some embodiments, the one or more supplements include insulin, which may be a growth supplement.

In some embodiments, the one or more supplements include laminin, which may be a growth enhancer of stem cells.

In some embodiments, the one or more supplements include a glutamine supplement, such as GlutaMAX (L-alanyl-L-glutamine dipeptide).

In some embodiments, the one or more supplements include chemically defined lipid concentrate (CDLC), which is a concentrated lipid emulsion. According to product information sheet, Gibco CDLC contains unsaturated fatty acids and a surfactant.

In some embodiments, the one or more supplements include amphotericin B or derivatives thereof.

In some embodiments, a cerebellar differentiating medium contains knockout serum replacement (KSR). In some embodiments, the KSR replaces fetal bovine serum, and so a cerebellar differentiating medium does not contain fetal bovine serum. In some embodiments, KSR is not used, and fetal bovine serum is used. In some embodiments, the KSR includes Glycine, L-histidine, L-isoleucine, L-methionine, L-phenylalanine, L-proline, L-hydroxyproline, L-serine, L-threonine, L-tryptophan, L-tyrosine, L-valine, Thiamine, reduced glutathione, ascorbic acid 2-PO₄, Ag⁺, Al³⁺, Ba²⁺, Cd²⁺, CO²⁺, Cr³⁺, Ge⁴⁺, Se⁴⁺, Br⁻, I⁻, Mn²⁺, Si⁴⁺, V⁵⁺, Mo⁶⁺, Ni²⁺, Rb⁺, Sn²⁺, Zr⁴⁺,Transferrin (iron-saturated), insulin, and lipid-rich albumin (AlbuMAX).

In some embodiments, the one or more supplements include apo-transferrin. Serum transferrin facilitates the transport and uptake of iron cells in culture. Apo-transferrin may be used to supplement culture media that are formulated with elevated levels of ionic iron, such as DMEM.

In some embodiments, one or more of N-2 supplement, B-27 supplement, amphotericin B (AmphoB), 3, 5, 3′-triiodo-L-thyronine (T3), Matrigel, among others, are added to supplement a cerebellar differentiating medium. For example, N-2 Supplement is a chemically defined, serum-free supplement based on Bottenstein's N-1 formulation. N-2 Supplement can be used as a substitute for Bottenstein's N-1 formulation. B-27 Supplement is an optimized serum-free supplement used to support the low- or high-density growth and short- or long-term viability of embryonic, post-natal, and adult hippocampal and other CNS neurons. T3 is an active, circulating thyroid hormone, while tetra-iodothyronine (T4) is believed to be the precursor of T3. Matrigel is rich in extracellular matrix proteins, including laminin (a major component), collagen IV, heparan sulfate proteoglycans, entactin/nidogen, and a number of growth factors.

In some embodiments, a cerebellar differentiating medium contains a basal medium comprising a mixture of one or more DMEM, F-12, and neurobasal medium. Neurobasal Medium is a basal medium typically used without the need for an astrocyte feeder layer when used with Gibco B-27 supplements. According to ThermoFisher (www.thermofisher.com/us/en/home/technical-resources/media-formulation.251.html), the neurobasal medium contains amino acids, vitamins, and inorganic salts, and other components including D-glucose, HEPES, and sodium pyruvate.

In some embodiments, the present invention includes a culture medium for deriving and maintaining neuronal lineage embryoid bodies, which is a growth factor-reduced medium. In some embodiments, a growth factor-reduced medium has zero or substantially zero growth factor. In other embodiments, a growth factor-reduced medium has more than 90%, 80% or 70% reduction in total growth factor amount compared to corresponding medium without the growth factor reduction. In some embodiments, all components in a growth factor-reduced medium (including concentrations) are known.

In some embodiments, a growth factor-reduced medium comprises or consists of a basal cell growth medium (e.g., IMDM/F12) at a volume percentage of about 80% to 100% (optionally 80% to 90%, 90% to 95%, 95% to 99%), a serum albumin supplement (e.g., bovine serum albumin) optionally at a percentage of 0.1% to 5% (optionally at about 1%, 2%, 3%, 4%), a chemically defined lipid concentration optionally at a percentage of 0.1% to 5%, apo-transferrin optionally at a percentage of 0.1% to 5%, a supplement to stimulate proliferation (e.g., mono-thioglycerol) optionally at a percentage of 0.1% to 5%, and a growth supplement (e.g., insulin) optionally at a percentage of 0.1% to 5%.

In some embodiments, the present invention includes one or more cerebellar differentiation media. In some embodiments, a cerebellar differentiation medium comprises or consists of a basal cell growth medium (e.g., DMEM/F12) at a volume percentage of about 80% to 100% (optionally 80% to 90%, 90% to 95%, 95% to 99%), supplemented with knockout serum replacement optionally at a percentage of 0.1% to 5%, apo-transferrin optionally at a percentage of 0.1% to 5%, a growth supplement (e.g., insulin) optionally at a percentage of 0.1% to 5%, a glutamine supplement (e.g., L-alanyl-L-glutamine dipeptide) optionally at a percentage of 0.1% to 5% (optionally 0.5% to 3%), and a reducing agent (e.g., 2-mercaptoethanol) optionally at a percentage of 0.05% to 0.5% (optionally at 0.1%).

In other embodiments, a cerebellar differentiation medium comprises or consists of a basal cell growth medium (e.g., DMEM/F12) at a volume percentage of 80% to 100% (optionally 80% to 90%, 90% to 95%, 95% to 99%), supplemented with 0.5% to 3% of N-2 Supplement, 0.5% to 5% of B27, and 0.5% to 3% L-alanyl-L-glutamine dipeptide.

In yet other embodiments, a cerebellar differentiation medium comprises or consists of a basal cell growth medium and a basal embryonic neuronal cell growth medium, combined at a percentage of 80% to 100% (optionally 80% to 90%, 90% to 95%, 95% to 99%), supplemented with 0.5% to 3% of N-2 Supplement, 0.5% to 5% of B27, and 0.5% to 3% L-alanyl-L-glutamine dipeptide, 0.1 to 5 g/ml of heparin, 0.5% to 5% of CDLC, antibiotics and antifungals (e.g., Amphotericin B) at a percentage of 0.5% to 3%. In various aspects, a basal embryonic neuronal cell growth medium is also a neurobasal medium.

In further embodiments, one or more cell culture medium may be added with (if not already present) any one or more of: non-essential amino acid (NEAA) supplement optionally at 0.5% to 3%, penicillin G and streptomycin optionally at 0.5% to 3%, and a reducing agent (e.g., β-mercaptoethanol) optionally at 0.05% to 0.5%.

Methods for cell culture can require agitation of the cells, for example, when the cells are grown in suspension or when scaffolds are used. Thus, in one example, an orbital shaker is used in the culturing.

In further embodiments, the methods may include continue culturing the generated cerebellum-like organoids for 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 36 months, 48 months, or more; and preferably the mature functional Purkinje cells in the cerebellum-like organoids maintain the biological activity and markers for the duration of the culture.

Various embodiments further provide cerebellum-like organoids, which may be prepared by one or more generation methods disclosed herein and demonstrated in Examples. In some embodiments, a cerebellum-like organoid is derived from human stem cells and contains spatially segregated ventricular zone progenitors and rhombic lip progenitors, and the cerebellum-like organoid contains cells expressing Purkinje cell markers. In some embodiments, the cerebellum-like organoid further comprises a cluster of Bergmann glial marker-positive cells, a cluster of neuronal cells, a cluster of interneuron precursors, a cluster of interneurons, a cluster of cerebellar nuclei, and glutamatergic neurons; the cerebellum-like organoid further comprising: choroid plexus or TTR+ cells, meninges or LUM+DCN+ cells, and roof plate cells or GDF7+ cells. In further embodiments, the ventricular zone progenitors are KIRREL2+, PTF1A+, and VIM+, the rhombic lip progenitors are ATOH1+ and BARHL1+, the Bergmann glial marker-positive cells express or are positive for PTPRZ1, EDNRB, GFAP, HOPX, SLCA4A4, and EDNRB, the cluster of neuronal cells comprises the cells expressing the Purkinje cell markers, the cluster of interneuron precursors express or are positive for PAX2, the cluster of interneurons express or are positive for SOX14 and DMBX1, the cluster of cerebellar nuclei express or are positive for MEIS2, LHX9, and IRX3, and the glutamatergic neurons express or are positive for STMN2+ and SLC17A7+, wherein the glutamatergic neurons comprise unipolar brush cells (UBC), granule cells (GC), and granule cell progenitors (GCP); or the glutamaterigic neurons comprise a cluster of cells expressing or positive for EOMES, a cluster of cells expressing or positive for NEUROD1 and NHLH1, and a cluster of cells expressing or positive for ATOH1 and BARHL1.

Various embodiments provide that cerebellum-like organoids can be used for toxicity and efficacy screening of agents that treat or prevent the development of a neurological condition.

In some embodiments, an organoid generated according to the methods described herein is contacted with a candidate agent. The viability of the organoid (or various cells within the organoid) is compared to the viability of an untreated control organoid to characterize the toxicity of the candidate compound. Assays for measuring cell viability are known in the art, and are described, for example, by Crouch et al. (J. Immunol. Meth. 160, 81-8); Kangas et at (Med. Biol. 62, 338-43, 1984); Lundin et al., (Meth. Enzymol. 133, 27-42, 1986); Petty et al. (Comparison of J. Biolum. Chemilum. 10, 29-34, 0.1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988).

In some embodiments, the organoid comprises a genetic mutation that effects neurodevelopment, activity, or function. Polypeptide or polynucleotide expression of cells within the organoid can be compared by procedures well known in the art, such as Western blotting, flow cytometry, immunocytochemistry, in situ hybridization, fluorescence in situ hybridization (FISH), ELISA, microarray analysis, RT-PCR, Northern blotting, or colorimetric assays, such as the Bradford Assay and Lowry Assay.

In some embodiments, one or more candidate agents are added at varying concentrations to the culture medium containing a cerebellum-like organoid. An agent that promotes the expression of a polypeptide of interest expressed in the cell is considered useful; such an agent may be used, for example, as a therapeutic to prevent, delay, ameliorate, stabilize, or treat an injury, disease or disorder characterized by a defect in neurodevelopment or neurological function. Once identified, agents of the invention may be used to treat or prevent a neurological condition. Exemplary neurological diseases or conditions include, but are not limited to, intellectual disability, autism spectrum disorder, spinocerebellar ataxia, Joubert Syndrome, and cerebellar malformation.

In other embodiments, the activity or function of a cell of the organoid is compared in the presence and the absence of a candidate compound. Compounds that desirably alter the activity or function of the cell are selected as useful in the methods of the invention.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Human Cerebellar Organoids (hCerOs) Reproducibly Generated the Cellular Diversity of the Human Cerebellum.

The cerebellum develops from the most rostral region of the rhombomere 1, an area of the developing neural tube just caudal to the isthmic organizing center, which defines the midbrain-hindbrain boundary. Modulation of key morphogen pathways has allowed for the establishment of protocols that differentiate 3D cultures of human pluripotent stem cells into organoids specific to particular brain regions, including the cerebellum. However, it has been shown that caudalization relying on only FGF2+insulin is inferior to other patterning strategies in generating a hindbrain fate. This is not surprising, as foundational neurodevelopmental biology studies have shown that the combinatorial activity of several caudalizing factors is required in vivo to overcome the preference of neural tissue to develop an anterior character. To develop a reproducible in vitro model of the cerebellum we established an organoid protocol based on the modulation of the signaling molecules required for the development of the isthmic organizer in vivo. To promote neuroectodermal differentiation toward a cerebellar fate, we treated cell aggregates simultaneously with two inhibitors of SMAD signaling, Noggin and SB431542, and the canonical WNT pathway activator CHIR-99021. On day 4, FGF8b was then added to specify the midbrain-hindbrain boundary and suppress midbrain fate, re-creating the isthmic organizing center seen in vivo.

The use of this patterning strategy along with the use of insulin and growth factor reduced media (gfCDM) led to the successful elimination of forebrain progenitors, as observed through the use of a forebrain reporter cell line (PGP::tdTOMATO::FOXG1). However, this protocol led to incomplete caudalization and yielded a midbrain-like tissue identity with the presence of BARHL1+ granule cell progenitors and a complete lack of SKOR2+ postmitotic Purkinje cell precursors, which was confirmed through increased expression levels of EN1, EN2, and PAX8 midbrain regional identity markers. It is known that base media can affect regional patterning. We thus created a cerebellar induction strategy based on an optimized combination of base media to lock in the midbrain/hindbrain boundary identity and to generate a pure compendium of cerebellar cell types. With this protocol, at day 30, the hCerOs reliably generated spatially segregated ventricular (KIRREL2) and rhombic lip (ATOH1+) progenitor zones, as well as their derivatives: excitatory BARHL1+ granule cell progenitors and newborn SKOR2+ inhibitory Purkinje neurons. Importantly, the ability to generate these two populations of neuronal progenitors was identified across three hiPSC lines including PGP1, 11a, and D2, and one hESC line, 1-9. These data indicated the potential of our protocol to generate all the cell types of the developing cerebellum across multiple hPSC lines.

To determine the cellular diversity developed within our hCerOs, we performed single cell RNA sequencing on 2-month-old organoids (n=14,947 cells from 3 individual organoids). We systematically categorized the clusters by comparing signatures of differentially expressed genes to a pre-existing dataset of endogenous cell types of the human developing cerebellum described by K. A. Aldinger et al., Nat Neurosci. 24, 1163-1175 (2021). Uniform Manifold Approximation and Projection (UMAP) dimensionality reduction identified 16 major clusters, including the two main progenitor types, the KIRREL2+, PTF1A+, and VIM+ ventricular zone progenitors, and ATOH1+ and BARHL1+ rhombic lip progenitors, which derive the inhibitory and excitatory neuronal populations. In vivo, the ventricular zone also gives rise to Bergmann glia, a scaffolding cell type necessary to aid in the migration of maturing granule cells into their proper orientation. Indeed, within our datasets, we were also able to identify a cluster of cells that expressed key Bergmann glial markers including PTPRZ1, EDNRB, GFAP, HOPX, SLCA4A4, and EDNRB. Among the neuronal clusters, we identified cells expressing in vivo canonical markers of Purkinje cells, including SKOR2, RORA, FOXP2, and CALB1. To our knowledge, this is the first time Purkinje cells with the transcriptional profile of their bona fide in vivo counterparts have been generated in vitro in an all-human system. In addition, we classified three other GABAergic (STMN2+, GAD2+) clusters: interneuron precursors expressing PAX2, molecular layer interneurons expressing SOX14 and DMBX1, and inhibitory deep cerebellar nuclei with markers MEIS2, LHX9, and IRX3. Additionally, the Purkinje cells and cerebellar nuclei had corresponding “immature” clusters, which had a similar transcriptomic profile as their more mature counterparts but lacked the expression of genes associated with later stages of maturation in vivo. These include CALB1, FOXP2, and RORA for Purkinje cells, and NEUROD1/2 for cerebellar nuclei. We also identified three types of glutamatergic neurons (STMN2+, SLC17A7+) expressing Unipolar Brush Cell (UBC) marker EOMES, Granule Cell progenitor (GCP) markers ATOH1 and BARHL1, and mature Granule Cell (GC) expressing higher levels of more mature neuronal markers including NEUROD1 and NHLH1.

Previously reported work has revealed that the human rhombic lip is divided into a ventricular and subventricular zone (RLvz and RLsvz), a feature that is not shared by any other vertebrates. Because of the human specific nature of our system, we then sought to identify these uniquely human rhombic lip progenitor subsets. To determine if the cell types specific to the RLvz and RLsvz are present in our system, we subclustered the RL and dividing VZ clusters, which were then integrated with 1018 cells identified to have RL identity in the human developing cerebellum dataset. We observed that our cells correlated well with the RLvz and RLsvz clusters of the human dataset, indicating that we have both populations of progenitors present in our organoids. We then took the DEGs from the human dataset used to characterize the RLvz, RLsvz, and the IZ. This comparison confirmed the transcriptomic similarity of hCerO-derived cells to distinct sub clusters found in the human dataset. We also saw the expression of classic RL markers, including MKI67, PAX6, and LMX1A throughout our dataset, as was seen in the human dataset. Additionally, the expression of WLS, SOX2, and CRAYAB was predominantly expressed in the RLvz cluster, while higher levels of expression for EOMES, DPYD, GPC6, OTX2, and BARHL1 were seen in the RLsvz.

In addition to the cell types deriving directly from the two progenitor zones of the cerebellum, we also observed the ability of hCerOs to co-develop other cell types also found in the human fetal dataset known to be important contributors to cerebellar development in vivo. These cell types include TTR+ Choroid Plexus (TOP2A+ dividing choroid plexus), LUM+DCN+ Meninges, and GDF7+ Roof Plate cells.

As an additional line of evidence, we characterized the regional identity generated within hCerOs in an unbiased manner through VoxHunt (github.com/quadbio/VoxHunt), a tool used to assess cell type composition and developmental stage of neural organoids. Using this tool, we mapped the 2-month-old hCerO's scRNA-seq data onto the BrainSpan human transcriptomic dataset and found that our dataset showed the highest scaled correlation with cerebellum (CB) when compared to primary brain samples between 10-25 post-conception weeks (PCW).

Finally, to assess organoid-to-organoid reproducibility in cell type composition, we performed scRNA-seq on three 2-month-old hCerOs (org1: 4,817 cells, org2: 6,768 cells, org3: 3,362 cells) and found that they individually generated an indistinguishable compendium of cell types.

Altogether, our data show that our hCerO protocol reproducibly generates all the major cell types of the developing cerebellum, including human-specific progenitor subtypes of the fetal rhombic lip and Purkinje cells that express the transcriptional profile of their bonafide counterparts.

hCerOs Display Organized Laminar Layering Reminiscent of the EGL and PCL of the Developing Cerebellar Anlage.

While analysis of the cell type composition of hCerOs demonstrated that the generation of cerebellar cellular diversity is an intrinsic process that does not require the in vivo cues of the developing embryo, we next investigated whether distinctive cytoarchitectural features could also organically emerge from this system. During the second trimester of human cerebellar ontogenesis, the excitatory granule cell precursors undergo directed tangential migration along the pial surface to form the external granule layer (EGL), meanwhile the Purkinje cells migrate radially toward the pial surface, settle below the EGL, and form the multicell Purkinje layer (PCL). Despite the presence of these neuronal populations, we found that this mechanism is not intrinsically regulated in 2-month-old hCerOs, a stage roughly equivalent to the second trimester of human fetal development. Thus, the 2-month-old hCerOs are unable to organize as seen in vivo. Therefore, we decided to instruct their migration by using external cues.

During fetal development, stromal cell-derived factor 1 (SDF1, or SDF-1; SDF-1 alpha (SDF1a); also known as CXCL12), a chemoattractant released by meningeal cells, guides the tangential migration of granule cell progenitors along the pial surface through the activation of the corresponding CXCR4 receptor. In agreement with in vivo expression patterns, we found that granule cells in hCerOs express CXCR4 in our 2-month-old hCerOs, thus indicating potential sensitivity to this chemoattractant. To assess the response of hCerOs to SDF1, we added 100 ng/ml of recombinant SDF1a into our maintenance media every 3 days (CerDM3) for 1 month (between day 30-60). After treatment, we observed the BARHL1+ and PAX6+ cells aligned adjacent to the edge of the organoid, while the SKOR2+ cells appeared beneath that layer. When co-staining for the Purkinje cell zone (CALB1+) and the RL-derivative zone (BARHL1+), we noticed that there appear to be two distinct layers in the +SDF1a treated hCerOs, reminiscent of the apical-basal polarity seen in the early stages of murine cerebellar development (˜E15). Interestingly, a previous attempt to modify the structural organization of cerebellar organoids using 300 ng/mL SDF1a for 7 days following FGF19 treatment, yielded a reserve organization of the cerebellar anlage as described in K. Muguruma et al., Cell Reports. 10, 537-550 (2015), indicating the importance of the fine tuning the duration and concentration of chemoattractants to build specific cytoarchitectrual features within organoid models. Altogether, we found that while laminar layering is not intrinsically encoded in developing hCerOs, administration of development-inspired external cues at the right concentration and timing, can aid in improving the ability of organoids to recapitulate features of the anatomical organization found in the developing human brain.

hCerOs Display Functionally Mature Network Activity in Long-Term Cultures, Resembling Patterns of In Vivo Cerebellar Circuits.

Following the characterization of the cellular diversity and cytoarchitectural features of hCerOs, we interrogated whether spontaneously active networks were present. We performed live two-photon microscopy-based imaging of intact 2-month-old hCerOs, following infection with AAV8 GCAMP6f virus to record intracellular calcium dynamics. By performing an unbiased analysis and categorization of single-cell calcium dynamics, we identified individual Tetrodotoxin (TTX, a sodium channel blocker) sensitive Ca2+ neuronal calcium signals within hCerOs, indicative of spontaneous neuronal activity. Importantly, we found that levels of spontaneous activity, as shown by the average activation per frame, amplitude, and duration of calcium events, were similar across organoids derived from 3 hiPSC lines: D2, PGP1, and 11a. This further indicates that hCerOs display a high degree of organoid to-organoid reproducibility, not only in terms of cellular composition, but also at a functional level.

In addition, the single cell calcium signals were then used to characterize brain organoid physiological activity at network levels. We performed clustering analysis and found the presence of multiple functional clusters spatially distributed across the entire organoid, indicating that as early as 2 months, neurons in hCerOs display coordinated firing indicative of the emergence of spontaneous network activity.

We then investigated whether long-term culture of hCerOs would lead to changes in network activity. s. comparison of 2- and 6-month-old hCerO neuronal spiking profiles showed that 6-month-old organoids had an overall increase in average activation per frame and decrease in interpeak time per neuron, indicating an increase in overall calcium transient activity and more neurons contributing to the activation of organoids. Additionally, we see an increase in the average correlation coefficient and increase in burstiness within clusters, indicating a transition from single mature neuronal events into spontaneous coordinated activity across multiple neurons.

As hCerOs contain a mixture of glutamatergic granule cells/eCN/UBC and GABAergic Purkinje cells/BLI/iCN/PIP cells, we sought to assess their distinct functional contributions through both application of N-methyl-D-aspartate (NMDA) and picrotoxin (PTX) to 2- and 6-month-old organoids. We found that combined treatment of 6-month-old hCerOs with NMDA+PTX greatly increased burstiness in each cluster, compared to treatment with NMDA alone. This indicates a contribution of both glutamatergic and GABAergic neurons to the overall activity within clusters. The level of activity post-treatment with NMDA+PTX is significantly higher than 2-month-old hCerOs, indicating an increase in the level of functional connectivity in long-term cultured hCerOs.

Overall, this data shows the emergence of functional network activity in hCeros after as little as 2 months in culture. Long-term culture increases the level of functional connectivity between excitatory and inhibitory neurons within hCerOs. The data provide, for the first time, a foundation for the use of human cerebellar organoids to model functional impairment associated with dysfunction in the development of cerebellar microcircuits.

Functionally Mature, Bona Fide Human Purkinje Neurons Develop within Long-Term Culture of hCerOs.

To our knowledge, functional maturation of bonafide (e.g., functional and mature) Purkinje cells has never been obtained in an all-human culture system prior to our invention. Therefore, we focused our investigation on the functionality of these neurons, which are dysfunctional in an array of neurological disorders. We identified Purkinje cells in 6-month-old hCerOs through the expression of classical in vivo marker genes, such as PCP2, DAB1, RORA, and FOXP2, via spatial transcriptomics, which also gave us access to the spatial localization of these neurons. Having identified these neurons on the outer edge of the organoids, we decided to conduct whole cell patch clamp recordings on intact hCerOs to prevent the disruption of the complex neuronal connections.

Given that PCP2 is differentially expressed within our Purkinje cells and has been used to identify Purkinje cells in live human culture systems, we decided to infect the hCerOs with a virus that fluorescently reports for the expression of PCP2. Following whole-cell patch-clamp recordings of Purkinje Cell Protein 2 (PCP2)+ neurons labeled with an AAV8.L7-6.eGFP.WPRE.hBG, we found that PCP2+ cells (n=19) at days 135-220 presented an average resting membrane potential (RMP) of −53.88 mV (table 9). 84.2% of the recorded cells showed the ability to fire multiple mature induced action potentials with an average amplitude of 68.80 mV. 42.10% of the recorded neurons presented the ability to fire spontaneous action potentials with an average frequency of 14.63 Hz, highlighting the mature profile of the recorded neurons. Importantly, as observed in cerebellar Purkinje neurons in vivo, we were able to detect the presence of hyperpolarization-activated current (Ih) current and repetitive spontaneous firing, a profile distinctive of Purkinje cells, in our PCP2+ cells.

Although postnatal mice glia and granule cells were previously deemed necessary for the functional maturation of hiPSC-derived Purkinje cells, our all-human system shows for the first time, the development of bonafide Purkinje cells that express classical in vivo Purkinje cell markers and display the distinct electrophysiological profile of their in vivo counterparts.

hCerO Cell Type Enrichment in Various Diseases

The cerebellum's protracted development makes it susceptible to early neurodevelopmental disorders, and it is also vulnerable to adult-onset degenerative diseases. To validate the fidelity of hCerOs as a viable in vitro model to assess the selective vulnerability of distinct cerebellar cell types to cerebellar disorders, we crossed our 2-month-old hCerO single cell dataset with genes associated with intellectual disability (ID), autism spectrum disorder (ASD), spinocerebellar ataxia (SCA), Joubert Syndrome, cerebellar malformations, Alzheimer's disease (AD). The gene lists associated with these disorders were obtained from Aldinger et al. Nat Neurosci. 24, 1163-1175 (2021).

First, we assessed gene enrichment for intellectual disability (ID) and found enrichment in nearly all neuronal cell types. In addition to finding enrichment within inhibitory neuronal clusters such as PIP, MLI, immature iCN, iCN, Purkinje Cells (PC), and immature PC, the ID gene set was also enriched in the excitatory granule cells (GC) with high levels of expression of SOX5, SATB2, and KCNQ3. Of the ID gene set, notable expression of a subset of these genes was observed within the MLI (EHMT1, ZC4H2, PPKAR1A, PPP1CB, QRICH1, KCNH1, U2AF2, NALCN, GNAI1, KCNB1, CHD2, CLTC, NSD1, CYP27C1, and GOLPH3).

Next, we examined the enrichment of autism spectrum disorder (ASD), a disorder known to result in structural and functional cerebellar abnormalities. The risk gene set was most significantly enriched in the neuronal cell types including PIP, molecular layer interneurons (MLI), inhibitory cerebellar nuclei (iCN), immature iCN, Purkinje Cells (PC), and immature PC with most notable levels of expression in MLI (TNRC6B, SMARCC2, FAM98C, CHD2, UBN2, DSCAM, TBL1XR1, CUL3), PIP (USP45, ERBIN, PYHIN1), immature PC and PCs (ASXL3, SHANK2, CACNA2D3, SLC6A1, PTEN, P2RX5, UIMC1), and immature CN and CNs (PAX5 and ACHE).

We also investigated spinocerebellar ataxia (SCA), which is defined by Purkinje cell loss and associated with movement control and muscle coordination issues. At this stage in development, we found notable levels expression of SCA risk genes (DAB1, KIF26B, and ITPR1) in the PC cluster including a significant enrichment of the SCA gene set in immature PC (TBP) and the MLI (NOP56, FGF14, ATXN8OS, and PRKCG).

We continued our examination with Joubert Syndrome, an autosomal recessive ciliopathy. The genes associated with this syndrome were significantly enriched in the choroid plexus and roof plate cell types with most notable levels of expression in TMEM231, B9D1, and NPHP1 in choroid plexus and CC2D2A in the roof plate cells.

Finally, we investigated the enrichment of genes associated with Dandy-Walker malformations and hypoplasia under the umbrella of cerebellar malformations. We see this gene set enriched significantly in PAX2+ interneuron precursors (PIP) with high levels of expression in PTF1A and EBF2. As expected, we found no significant enrichment in any of the genes associated with Alzheimer's disease, a progressive disease leading to adult-onset cerebellar atrophy. Together, these data demonstrate the ability of the hCerOs to uncover the cell type specific mechanisms underlying these and other disorders stemming from dysfunctions of early human cerebellar development.

Overall, our study establishes an organoid model to investigate the cellular interactions that orchestrate development, homeostasis, and diseases of the human cerebellum. This is enabled by the establishment of a protocol that reproducibly generates all the major cell and supporting cell types that aid in overall cerebellar development, including choroid plexus and roof plate cells. This diverse compendium of cell types includes populations that have never been generated in vitro prior to this study, including a human-specific progenitor subtype of the Rhombic Lip (RLsvz) and fast spiking interneurons of the molecular layer (MHLIs).

The generation of the RLsvz indicates that hCerOs can be leveraged to advance understanding of cerebellar evo-devo biology and of diseases that have been shown to stem from dysfunction in rhombic lip development, including Dandy Walker Syndrome and pediatric cancers. Additionally, MLIs play an important role in cerebellar circuitry by shaping the spike activity of Purkinje cells. Therefore, the presence of these cells within hCerOs could contribute to creating an environment that aids in the functional maturation of bonafide Purkinje cells, which has previously only been achieved through the co-culture of mouse glia and granule cells (K. Muguruma et al., CellReports. 10, 537-550 (2015); M. Sundberg et al., Mol Psychiatry. 23, 2167-2183 (2018); D. E. Buchholz et al., Proceedings of the National Academy of Sciences. 117, 15085-15095 (2020)). While Purkinje cells are the main cell type affected in a large number of neurodegenerative disorders, it is becoming increasingly evident that interaction with other cell types, including but not restricted to glia cells, may play a central role in disease pathogenesis. Therefore, hCerOs will transform our ability to model these disorders with high fidelity in an all-human system.

As the link between cerebellar dysfunction and neuropsychiatric connectopathies, including ASD and ID, becomes more evident, the generation of multiregional organoids that can model long-range connections within the patients' brains becomes a priority. In this scenario, hCerOs with bonafide Purkinje cells, the main neuronal output of the cerebellum, will enable the recapitulation of cerebellar connectivity with other brain regions and accelerate therapeutic discovery.

### Techniques and Procedures

### Pluripotent Stem Cell Culture

The PGP1 (Personal Genome Project 1) human iPS cell line was from the laboratory of G. Church; PGP1-FOXG1 human iPS cell lines were obtained from Harvard Stem Cell Institute; the H9 human ES cell line was purchased from WiCell, the 11a human iPS cell line was from the laboratory of Kevin Eggan, and the D2 human iPSC cell line was from the laboratory of Marcelo Coba. All stem cell lines were cultured on Geltrex (Gibco)-coated tissue culture plates, using mTeSRlmedium (Stem Cell Technologies) with 100 U/ml penicillin and 100 μg/ml streptomycin (Corning) at 37° C. in 5% CO₂. All human stem cells were maintained below passage 50 and periodically karyotyped via the G-banding Karyotype Service at Children's Hospital Los Angeles and were negative for mycoplasma (MycoAlert Plus Mycoplasma Detection Kits).

### Organoid Differentiation

For differentiation, feeder-free cultured human stem cells, 80% confluent, were dissociated to single cells using room temperature Accutase (Sigma) and were reaggregated in ultra-low-attachment, V-bottomed, 96-well plates (Sbio) at 6,000 cells per 100 ul per well, in a growth factor reduced chemically defined medium (gfCDM), consisting of 1:1 of Iscove's Modified Dulbecco's Medium (IMDM) (Gibco) and F-12 (Gibco), 5 mg/ml >99% Bovine Serum Albumin (BSA) (Sigma), 1% chemically defined lipid concentrate (CDLC) (Gibco), 15 ug/ml Apo-transferrin, and 450 uM Mono-Thioglycerol (Sigma) with 7 ug/ml insulin (Sigma), 10 μM of SB431542 (Selleckchem), 50 ng/ml Noggin (Peprotech), and 1.7 μM CHIR (Reprocell). On Day 2, the concentration of Noggin was increased to 100 ng/mL by removing 40 uL of the media and adding 50 uL containing 150 ng/ml Noggin in addition to the same concentration of the previous morphogens. To initiate the specification of the midbrain/hindbrain boundary, 40 ul of media was removed and 50 ul of 200 ng/mL FGF8b was added to each well to bring the final concentration in the well to 100 ng/mL. A rho-associated, coiled-coil containing protein kinase (ROCK) inhibitor (ROCKi) was added on the day of seeding (Day 0) at a final concentration of 20 μM and once again at 20 μM on Day 2. Although SB431542, CHIR, and Noggin were still added at the same concentration on day 4, ROCKi was no longer added on day 4 and beyond to promote cell proliferation and aggregate growth. To promote the cerebellar cell fate, on Day 6, 40 ul of media was removed and 50 ul of fresh cerebellar differentiation medium (CerDM) I, containing DMEM/F-12 (Corning/Cytiva), 20% Knockout Serum Replacement (KSR) (Gibco), 15 μg/ml Apo-Transferrin (Sigma), 7 μg/ml Insulin (Sigma), 2 mM Glutamax (Gibco), 0.1 mM 2-Mercaptoethanol (Gibco), 100 U/ml penicillin and 100 μg/ml streptomycin (Corning) was added to each well while supplementing the media with the same final concentration of morphogens as in Day 4 (i.e., 10 μM SB431542, 100 ng/mL Noggin, 1.7 μM CHIR, 100 ng/mL FGF8b). On day 8, the final concentration of FGF8b was increased from 100 ng/ml to 300 ng/ml to push further toward the midbrain/hindbrain specification and 100 ul of media was added to each well after the removal of 40 ul taking the final volume to 150 ul. On Day 10, 80 ul of media was removed and supplemented with 100 ul of media with the same final concentration of the drugs from Day 8. On day 12 and 14, half media change was conducted without any drugs by removing 70 ul of media and adding 75 ul of fresh CerDM1.

From day 16 to day 30, organoids were cultured in ultra-low-attachment 10-cm culture plates (Falcon) with orbital agitation (10 r.p.m) in CerDM II, containing DMEM/F12 medium (Corning), 1% N2 (Gibco), 1% B27 (Gibco), 2 mM Glutamax (Gibco), 100 U/ml penicillin and 100 μg/ml streptomycin (Corning) and full media change was conducted every 3 days. This media was specifically formulated to help with neuronal survival (N2 and Glutamax) and growth (B27), which is a simplified and more cost efficient version compared to CerDMIII below.

From day 30 to day 60, organoids were cultured in CerDM III, consisting of DMEM/F12 (Corning) and Neurobasal medium (Gibco), 1% N2, 1% B27, 2 mM Glutamax, 5 μg/ml Heparin (Sigma), 1% Chemically Defined Lipid Concentrate (Gibco), 100 U/ml penicillin, 100 μg/ml streptomycin (Corning), 0.25 μg/ml AmphoB (Gibco), 0.5 ng/ml T3 (Sigma) and 1% Matrigel (Corning). CerDM III still helps with neuronal survival and growth, but compared to CerDM II, CerDM III includes additional reagents that are cofactors (heparin) of proliferative morphogens, lipids to help with neuronal function, T3 for purkinje cell survival, AmphoB for preventing potential fungal infections due to it being a long-term culture, and Matrigel to help with organization of the neurons. This stage of culture is where the Purkinje cells start to become more fragile and need support to survive long term and thus necessary to add a thyroid hormone (e.g., T3).

From day 60 onwards, organoids were cultured in CerDM IV, consisting of CerDM III without Matrigel and supplemented with 14 ng/ml Brain Derived Neurotrophic Factor (BDNF) (R&D Systems). Full media change was conducted once a week on organoids after day 30. This stage aids neuronal differentiation survival for long term culture, hence the addition of BDNF. Without BDNF, the survival of the neurons in just CerDMIII media (without BDNF) may be limited. Matrigel is no longer added since only adding Matrigel (without other signaling cues) does not aid in structural support or organization.

### Quantitative PCR

Organoids were lysed and the RNA was collected using the RNeasy Mini kit (Qiagen, #74004). Template cDNA was prepared by reverse transcription using the RevertAid First Strand cDNA Synthesis Kit (Thermo Fisher Scientific, #K1621). qPCR was performed using the SYBR Green PCR Master Mix (Thermo Fisher Scientific, #4309155) on a ViiA7 Real-Time PCR System (Thermo Fisher Scientific). Primers used in this study are listed in Table 9.

### Immunohistochemistry

Organoids were fixed in 4% paraformaldehyde for 30 min at room temperature before overnight incubation at 4° C. in 30% sucrose solution. Organoids were then embedded in Tissue-Tek O.C.T. compound (Sakura, #62550) and cryosectioned at 20 μm thickness onto glass slides (Globe Scientific, #1354W). Slides were washed 3× with a 0.1% Tween20 (Sigma #P9416) solution before a 1-hour incubation in 0.3% TritonX-100 (Sigma, #T9284) and 6% bovine serum albumin (Sigma, #AA0281) solution. Slides were incubated for 1-hour at room temperature in a primary antibody solution followed by a 1-hour room temperature incubation in a secondary antibody solution, both consisting of 0.1% TritonX-100 and 2.5% BSA with 3 washes before and after secondary antibody incubation. Slides were coverslipped using Fluoromount G (EMS, #50-259-73). Primary antibodies and dilutions used are specified in Table 10.

### Microscopy and Image Analysis

Organoids in culture were imaged using a Leica DMIL LED microscope (Leica). Immunofluorescence images were taken with a Leica Model TL LED Thunder widefield scope (Leica) and analysed using ImageJ software and MATLAB software.

### Calcium Imaging

Organoids were transduced with pAAV-CAG-SomaGCaMP6f2 (Addgene, #158757) using 1.5 μl of virus in 150μ of CerDM3 medium overnight on static condition. One day after infection, a full media change was performed using CerDM3 media. A full media change to BrainPhys Medium (STEMCELL Technologies, #05792) was performed on day three after transduction. Organoids were kept for at least one week in culture before the imaging session. Organoids were randomly selected and transferred to a recording chamber kept at 37° C. using a heating platform and a controller (TC-324C, Warner Instruments) in BrainPhys Optimized Medium (STEMCELL Technologies, #05796). Imaging was performed using a SP-8X microscope with a multiphoton laser. Time-lapse images were acquired at 1 frame for 860 ms, using a 25×0.95 NA water objective (2.5 mm WD) and resulting in a view of 200×200 μm². All imaging conditions including excitation light intensity, camera sensor gain, and exposure time were identical for all calcium imaging experiments.

Basal activity was recorded for 10 mins of the imaged organoid. Pharmacological treatment was performed with a bath application of Tetrodotoxin, TTX (Tocris, #1078/1), at a final concentration of 2 μM, 3 μM NMDA (Tocris, #0114) and 100 μM PTX (Hello Bio, #HB0506). Raw tiff calcium imaging files were analyzed using the CNMF and CalmAN package (CaImAn an open-source tool for scalable calcium imaging data analysis), to identify fluorescent transients and spike estimation in MATLAB (MathWorks). Calcium traces were plotted as relative scaled height in function of time. Hierarchical clustering and pairwise correlation was performed, with Correlation, linkage parameter was set to 1.5.

### Whole-Cell Patch-Clamp Recording

Organoids were transduced with AAV8.L7-6.eGFP.WPRE.hBG (Addgene, #126462) using 1.5 μl of virus in 150 μl of CerDM3 medium overnight on static condition in a single well of a 24 well low attachment plates. One day after transduction, a full media change was performed using CerDM3 media. On day three after transduction organoids were transferred to a 6 well low attachment plate and a full media change to BrainPhys Medium (STEMCELL Technologies, #05792) supplemented with NeuroCult SM1 Neuronal Supplement (STEMCELL Technologies, #05711) was performed. Organoids were kept for at least one week in culture before the recordings. Prior to recording, each organoid was transferred into 35 mm petri dishes (Ibidi, #80136) on a 10 μl geltrex drop (Thermo Fisher Scientific, #A1413301) and let for 15 mins in a 37° C. incubator. Afterwards, organoids were incubated in BrainPhys Optimized Medium (STEMCELL Technologies, #05796) until recording. PCP+ neurons were visualized under a fluorescence microscope (Olympus BX51 WI). Recordings were performed at RT. Multi-clamp 700B (Molecular Devices) was used for recordings and signals were acquired at 10 kHz using pClamp10 software and filtered at 2 kHz for voltage clamp recordings. Data acquisition was performed with Digidata 1440A (Molecular Devices). Borosilicate glass capillaries were used for patch pipettes ranging between 4.5-8 MOhm. Patch pipettes were filled with intracellular solution (in mM): 122.5 potassium gluconate, 12.5 KCl, 0.2 EGTA, 10 Hepes, 2 MgATP, 0.3 Na3GTP and 8 NaCl adjusted to pH 7.3 with KOH. Intracellular solution was kept on ice during recordings. Passive properties of the membrane were monitored immediately after break-in in current clamp mode. Membrane potential was kept at −70 mV and currents were injected from −100 pA to +100 pA with 10 pA increments to induce action potentials. Inward sodium and delayed rectifying potassium currents were measured in voltage clamp at depolarising steps of 10 mV. Recording data were analyzed by using pClamp 11 software (Molecular Devices). Baseline gap free signals acquired in current mode were manually adjusted and frequency calculated with automated event detection with event rejection set to 1 ms and 67.26 dV. Gap free traces acquired in voltage clamp mode were used to monitor postsynaptic currents and filtered with a low-pass 2000 Hz Gaussian filter during post hoc traces analysis.

### Dissociation of Cerebellar Organoids and Single-Cell RNA-Seq

Individual brain organoids were dissociated into single cells using the Worthington Papain Dissociation System kit (Worthington Biochemical). To give an estimated recovery of 6,000 cells per channel dissociated cells were resuspended in ice-cold PBS containing 0.04% BSA at a concentration of 1,000 cells/μl, loaded onto a Chromium Single Cell 3′ Chip (10×Genomics) and processed through the Chromium controller to generate single-cell gel beads in emulsion (GEMs). scRNA-seq libraries were prepared with the Chromium Single Cell 3′ Library & Gel Bead Kit v.2 (10× Genomics). Libraries from different samples were pooled based on molar concentrations and sequenced on a NextSeq 500 instrument (Illumina) with 26 bases for read 1, 57 bases for read 2 and 8 bases for index 1. After the first round of sequencing, libraries were re-pooled on the basis of the actual number of cells in each and re-sequenced to give an equal number of reads per cell in each sample and to reach a sequencing saturation of at least 50% (in most cases >70%).

### Single-Cell RNA-Seq Data Analysis

Bioinformatic analysis was performed using the Seurat R package. Briefly, all single cell transcriptomes from 3 organoids were combined, and a set of highly variable genes was determined. The initial PCA analysis was performed using this gene set and the number of statistically significant principal components (PCA) was identified via Jackstraw analysis. Cluster structures were remapped to two dimensions using Uniform Manifold Approximation and Projection (UMAP). Finally, a graph-based clustering approach was used to cluster the cells to produce putative cell-type clusters and return sets of differentially expressed marker genes for each cluster. These were compared to known marker genes in order to ascribe a cellular identity to each cluster. This data was then integrated with the human dataset to confirm cell type identifications.

### Spatial Transcriptomics

seqFISH

RNA expression data from multiple sections from two 6-month-old organoids were obtained on the Spatial Genomics platform. Briefly, sections were hybridized overnight at 37° C. with probes against a set of RNA transcripts for Purkinje cell and other cell type specific markers. Samples were washed the following day and loaded onto the automated Spatial Genomics microscope for imaging. Threshold values were manually chosen to select transcript dots with signal above background. Cells were segmented based on expanding the nuclear DAPI stain. Cell-by-gene count matrices were then used for further downstream analyses. Raw counts of transcripts were used for all analyses.

### Single-Cell Pre-Processing

The cell-by-gene matrix of counts was pre-processed using the scanpy pre-processing module. For sample 1, cells with less than 1 or greater than 1500 transcript counts were removed. For sample 2, cells with less than 30 or greater than 1500 transcript counts were removed; the higher minimum count threshold was used to remove likely necrotic cells in the center of the organoid.

### Identification of Purkinje Cells

Purkinje cells (PCs) were identified using CA8 expression. Cells with carbonic anhydrase 8 (CA8) counts greater than or equal to the 99th quantile were defined as PCs; cells with zero CA8 counts were defined as non-PCs and cells with CA8 counts between zero and the 99th quantile were defined as ambiguous.

### Differential Expression Between Purkinje and Non-Purkinje Cells

We tested for differential expression of gene transcripts between Purkinje cells and non-Purkinje cells as identified above. Ambiguous cells were removed. To avoid psuedo-replication we aggregated cellular transcript counts within samples prior to fitting a gamma-Poisson generalized linear model with glmGamPoi (C. Ahlmann-Eltze et al., doi.org/10.1101/2021.06.24.449781). We accounted for the difference in cell size between Purkinje cells and non-Purkinje cells by dividing cell counts by cell area prior to aggregation. Total transcript counts are often included as a size factor in RNA-seq based pseudo-bulk analysis. This accounts for variation in sequencing depth and cell count per sample. However, Spatial Genomics transcript counts are image based and therefore transcripts are not resampled. Size factors for the model were the number of cells per pseudo-bulked sample.

### Example 2. Generation and Maturation of Human Cerebellar Organoids from Pluripotent Stem Cells

Provided herein includes a human organoid model (hCerOs), derived from human pluripotent stem cells, which is capable of generating complex cellular diversity of the fetal cerebellum as well as distinct cytoarchitectural features. This method can be implemented in standard tissue culture rooms and give rise to Purkinje Cells, granule cells, human-specific rhombic lip subventricular zone progenitors among others within 1-2 months in culture. Modulating the basal media composition within the first two weeks can allow for the generation of different proportions of inhibitory and excitatory neuronal progenitor populations. Culturing of hCerOs for 6 months allows for healthy survival and maturation of Purkinje cells that display molecular and electrophysiological hallmarks of their in vivo counterparts, addressing a long-standing challenge in the field. Furthermore, as these organoids can be maintained for more than 1 year in long-term culture, they have the potential to model later events.

The emergence of human organoid model systems to study brain development and disease has the potential to pave the way toward answering the most challenging questions in neurobiology. With limited access to human tissue at early developmental stages, organoids provide a strong foundation to begin studying human development in a way that animal models or 2D in vitro culture systems cannot fully recapitulate. Organoids that generate a forebrain-like regional identity have already been well established. These models have proven essential for a deeper understanding of early human forebrain development and disease. However, with increasing evidence on the cerebellum's role in higher order cognitive function emerging, there is a need to model this region of the brain in an all-human system. This will allow for a more thorough understanding about the contributions of these different brain regions within a healthy and diseased state.

To develop a reproducible 3D in vitro model of the cerebellum we established an organoid protocol that addresses three main steps: (i) the patterning of hPCS to resemble an isthmic organizer-like environment, (ii) expansion of inhibitory and excitatory cerebellar progenitor pools, and (iii) the maturation of cerebellar neural cell types.

In order to generate cerebellar cells in vitro, our first goal was to understand the intricate signaling that occurs in vivo to establish a cerebellar identity within the developing neural tube. Cerebellar development is initiated by a series of patterning cues that specify the alar lamina of the midbrain/hindbrain boundary from which both inhibitory and excitatory populations of the cerebellum derive from. To obtain these cell types, we mimicked the signaling cues in conjunction with various media formulations to model this region in vitro.

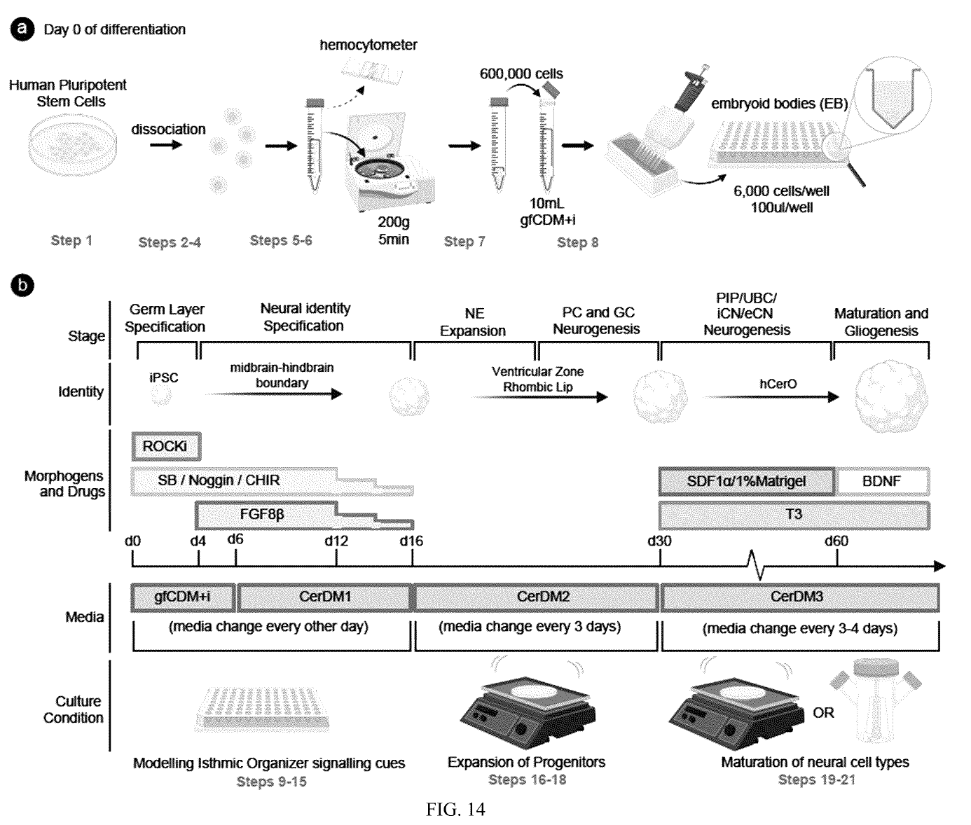

The first step toward modeling the midbrain/hindbrain boundary in hPSC cultures involves patterning towards neuroectoderm and further caudalizing this neuroectoderm. Previous studies have shown the rapid and efficient generation of neuroectoderm through inhibition of the SMAD pathways of TGFB and BMP (dual SMAD inhibition). However, without any caudalization factors, the hPSCs will automatically adopt a forebrain-like cellular identity. Therefore, we needed to expose the hPSCs to various morphogens to caudalize the cells toward a hindbrain fate. Key morphogen gradients within the developing neural tube, which govern brain region specification, include molecules that regulate the BMP, RA, WNT, and FGF pathways. Given that the WNT family of proteins have been identified as a main caudalization factor within the developing neural tube, we applied a canonical WNT agonist (CHIR-99021) to promote caudalization of these neural progenitors. However, to generate the most rostral region of the hindbrain (rhombomere 1) from which the cerebellar cell types derive from, we modeled the signaling cues from an adjacent organizing center at the midbrain/hindbrain boundary known as the isthmic organizer (IsO) (FIG. 14).

The isthmic organizer is one of many “organizing centers” with a defined genetic profile that specifies the neighboring neuroectodermal identity along the dorsal-ventral and rostro-caudal axes through morphogenic regulation. This specific organizer is identified at the midbrain/hindbrain boundary of the neural tube with high levels of expression of FGF8 once the strict boundary between the mesencephalon (OTX2+ midbrain) and metencephalon (GBX2+ hindbrain) is formed. FGF8 specifies this boundary while working in conjunction with other signaling molecules such as WNT1, which is constrained to the most caudal portion of the OTX2+ midbrain, adjacent to the isthmic organizer; and loss of function experiments of FGF8 yielded a complete loss of the cerebellum and tectum. Therefore, we included the use of FGF8 into our protocol. FGF8b is also believed to have the ability to convert cells destined toward a midbrain fate into hindbrain. As such, we used FGF8b and increased its concentration from 100 ng/mL to 300 ng/mL on Day 8 of the differentiation.

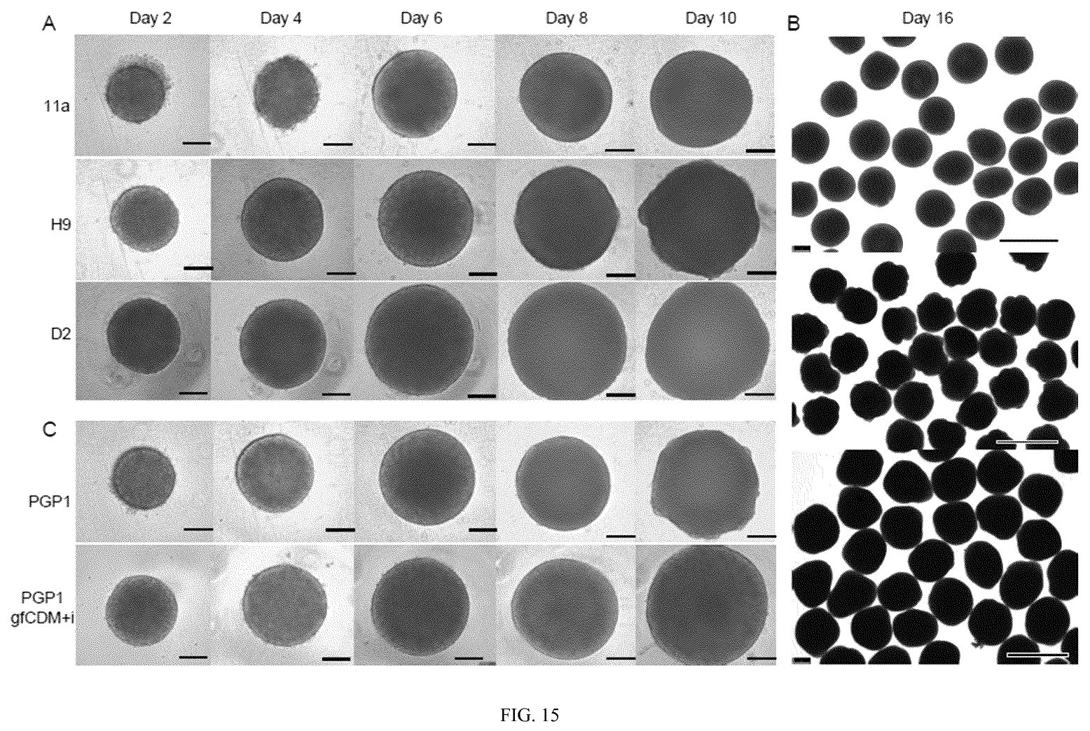

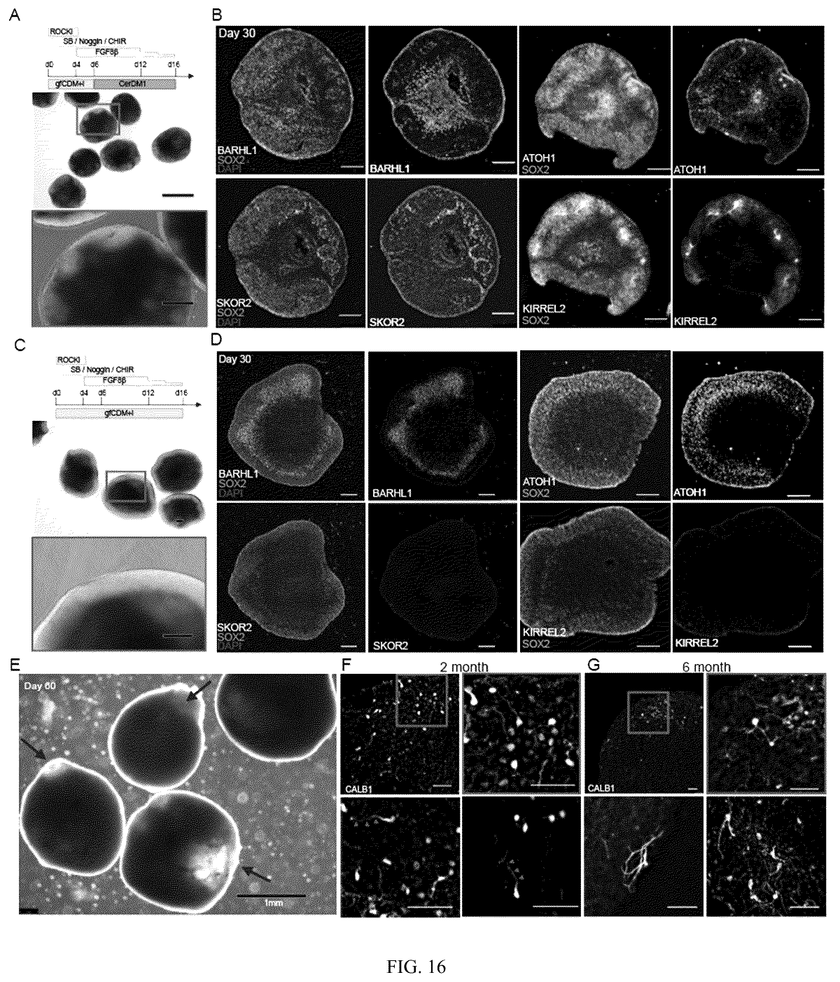

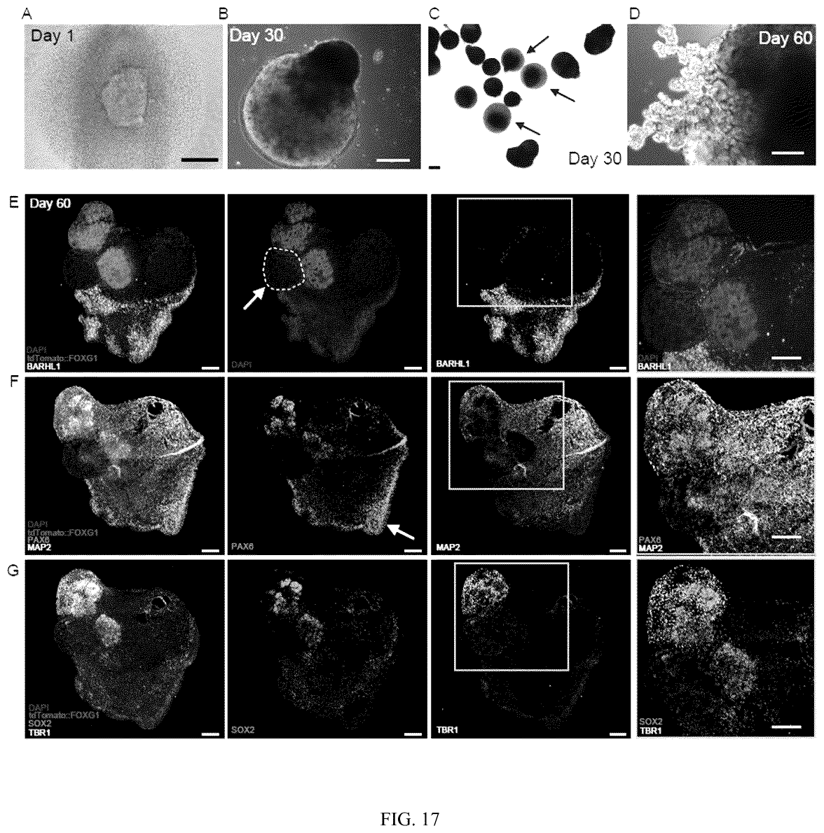

One interesting finding was the ability to modulate the cellular composition of the organoids simply by switching the basal media in which the organoids were cultured in without any alteration to the small molecule and patterning factor cocktail. Without the change to CerDM1 on Day 6 of the differentiation, the organoids cultured in gfCDM+i for 16 days generated only the excitatory neurons of the cerebellum (FIG. 15).

After 16 days in the 96-well, V-bottom plates where the organoids are exposed to isthmic organizer-like conditions, we transferred the organoids into 10 cm shaking culture conditions to expand the neuroepithelium and continue with the organoid maturation. By day 30, these organoids started to generate both inhibitory and excitatory neurons of the developing cerebellum, both of which derived from this established isthmic organizing-like center in vitro.

The protocol described here can be used in an array of studies that examine questions relating to human cerebellar development and disease. More importantly, this system can give us insight into the human specific traits that cannot be studied in other model systems. For example, it has been shown that the human rhombic lip possesses a human specific subventricular zone, which is not found in mice and other primates. Therefore, this human cerebellar organoid model system provides a platform to study abnormal development of the human rhombic lip, which has been associated with various disorders including Dandy-Walker malformations, cerebellar vermis hypoplasia, and medulloblastoma. More specifically, it has recently been shown that medulloblastoma, the most common childhood brain tumor, originates from the human specific, rhombic lip subventricular zone progenitor cells further emphasizing the need for a human based model system. The migration of the rhombic lip derivatives can also be tracked within this system as they move toward the edge of the organoid when treated with the chemoattractant for the CXCR4 receptor, SDF1a. This can bring deeper insight into the migration dynamics of these excitatory progenitors in the way that has been extensively conducted for migrating interneurons from the MGE and CGE. Additionally, this system has the potential to more physiologically model human specific disease phenotypes such as the loss of Purkinje cells in patients with ataxia telangiectasia, which is not observed in murine models of this disease. By doing so, human cerebellar organoid can also potentially be a better platform to study ways to address these phenotypes through various interventions and drug screenings. Therefore, this can be a platform to examine the emergence of human cerebellar diseases and its phenotypes, which can aid in developing more precise and targeted therapies.

To address potentially an issue of stressed core of the organoid (e.g., limited nutrient and oxygen delivered to the core), we cultured the organoid(s) under shaking/bioreactor conditions. However, unlike the forebrain organoids, which undergo a rapid expansion of the radial glial population leading to a dramatic increase in size, cerebellar organoids remain relatively smaller and therefore do not have as extensive of a necrotic core as cortical organoids do. We also administered SDF1a to aid in the organization as an exogenous guidance cue. In some embodiments, climbing fibers and mossy fibers are introduced by other co-culturing methods with the cerebellar organoids herein. In some embodiments, isogenic cell lines are used to generate the cerebellum-like organoids; which may limit cell line variability.

Compared with previous methods to generate cerebellar organoids, our work aims to develop Pukinje cells and incorporates 3D tissue culturing systems. We initially plate our hPSCs into low-attachment V-bottom plates in the presence of ROCKi to aid in cell survival. In our protocol, these cells self-aggregated and were simultaneously treated with the dual SMAD inhibitors, SB43152 and Noggin, as well as the canonical WNT pathway activator, CHIR-99021, to promote neuralization and commence caudalization. On Day 4, the aggregates are exposed to FGF8b to specify the midbrain-hindbrain boundary and suppress midbrain fate and re-creating the isthmic organizing center as seen in vivo. In addition to the morphogens used to pattern our organoids towards a cerebellar fate, our basal media composition during the first 16 days has been optimized to obtain both inhibitory and excitatory neurons. In the following days, our culture media includes the pro-neuronal B27+vitA component as well as shaking culture conditions on an orbital shaker to promote improved exchange of oxygen and nutrients delivery to the organoid. After 30 days in culture, our organoids are cultured in a serum-free media that contains lipids and heparin and is based on a combination of previously published brain organoid long-term culture techniques that allow for the growth and maturation of neurons over the course of many months. This media also includes BDNF and T3 for neuronal survival and maturation. By day 30, these cerebellar organoids resemble aspects of cerebellar development including the spatially segregated excitatory and inhibitory progenitor populations as well as their corresponding neuronal derivatives.

Overall this protocol has the ability to develop the fetal cerebellum's cellular diversity including Purkinje cells that display functional features of their in vivo counterparts. It seems that in previous studies the use of FGF2+insulin in addition to other morphogens such as BDNF and GDNF in long term cultures does not mature neuronal firing profile of the Purkinje cells enough to resemble the firing profile found in vivo. Additionally, most of previous studies required the use of mice granule cells and glia; whereas our methods do not require these mouse cells and still are able to achieve a level of maturation that allows these in vitro neurons to display the electrophysiological feature of a multi-peak firing profile specific to Purkinje cells. Additionally, a single-cell transcriptomic study of the FGF2+insulin patterning strategy has shown that these “Purkinje cells” from previous studies do not express the canonical markers of the developing Purkinje cell. This work reveals that our cerebellar organoids, within an all-human system, generate neurons that not only retain the electrophysiological properties of in vivo Purkinje cells, but also exhibit the canonical markers of Purkinje cells at the single-cell transcriptomic level.

Our protocol initiates with the maintenance of pluripotent stem cells in feeder-independent conditions. Following stem cell maintenance, the cells are dissociated from the plates and aggregated in into small spheres and cultured in various media conditions to (1) generate the isthmic organizer, (2) expand the progenitors, and (3) mature the neuronal cell types.

We have differentiated hESC and hiPSC lines in feeder-independent conditions. hPSCs are initially dissociated to single-cells and reaggregated in a low attachment V-bottom plate. Other devices such as low attachment U-bottom plates or AGGREWELL microwell plates may be alternatively used. The embryoid bodies (EBs) are simultaneously exposed to various morphogens that select for neuroectodermal development and followed by FGF8b to specify the midbrain-hindbrain boundary. At this stage, the media composition affects the cell types generated in these cultures. Following the static, suspension culture of aggregates in 96-well V-bottom plates, the EBs are then transferred to orbital shaking condition or spinning bioreactors to aid in oxygen and nutrient exchange within the organoids for long term growth and maturation.

Once generating cerebellar organoids, downstream assays including immunohistochemistry staining (IHC), calcium imaging, and Patch-clamp recording can be performed to assess the organoids.

TABLE 1
Growth media and supplements.

| | | |
|---|---|---|
| AmphoB | Gibco | 15290018 |
| Apo-transferrin | Sigma | T1147-100MG |
| BONE | R&D Systems | 248-BDB-050 |
| BrainPhys Optimized Medium | STEMCELL | 05796 |
| | Technologies | |
| Bovine Serum Abumin (BSA) (fatty | Sigma | A0281 |
| acid free and globulin free) | | |
| BSA (for all else) | | |
| B-27 + vitA supplement | Gibco | 17504001 |
| chemically defined lipid concentrate | Gibco | 11905031 |
| (CDLC) | | |
| CHIR | Reprocell | 04-0004 |
| DMEM/F-12 | Corning/Cytiva | SH30023.FS |
| FGF8b | Peprotech | 100-25 |
| Glutamax | Gibco | 35050061 |
| Ham's F-12 medium | Gibco | 11-765-054 |
| Heparin | Sigma | H314-25KU |
| IMDM medium | Gibco | 12440053 |
| Insulin | Sigma | 19278-5ML |
| Knockout Serum Replacement (KSR) | Gibco | 10828028 |
| Mono-Thioglycerol | Sigma | M1753 |
| mTeSR1medium | Stem Cell | 4309155 |
| | Technologies | |
| 5X supplement | | |
| Neurobasal medium | Gibco | 21103049 |
| NeuroCult SM1 Neuronal Supplement | STEMCELL | 05711 |
| | Technologies | |
| NMDA | Tocris | 0114 |
| Noggin | Peprotech | 120-10C |
| N2 supplement | Gibco | 17502001 |
| ROCK inhibitor | STEMCELL | 72302 |
| | Technologies | |
| SB431542 (TGFBi) | Selleckchem | S1067 |
| SDF1a | Peprotech | 300-28A |
| T3 | Sigma | T2877-250MG |
| 2-Mercaptoethanol | Gibco | 21985023 |

TABLE 2
Enzyme and other reagents

| | | |
|---|---|---|
| Accutase | Sigma | 07922 |
| DMSO | | |
| Fluoromount | EMS | 50-259-73 |
| Geltrex | Thermo Fisher Scientific | A1413301 |
| Growth-factor reduced Matrigel | Corning | 354230 |
| Trypan blue | | |
| Papain Dissociation solution | | |
| Paraformaldehyde | | |
| Penicillin/streptomycin | Corning | SV30010 |
| PTX | Hello Bio | HB0506 |
| Sucrose | | |
| SYBR Green PCR Master Mx | Thermo Fisher Scientific | 4309155 |
| Tetrodotoxin, TTX | Tocris | 1078 |
| Tissue-Tek O.C.T. compound | Sakura | 62550 |
| TritonX-100 | Sigma | T9284 |
| Tween20 | Sigma | P9416 |

### Exemplary Equipment:

CO2 incubators (Thermo Scientific, cat. no. 51026280)
Biological safety cabinet (Thermo Scientific, cat. no. 51022482)
Rainin Pipet Plus (P 1000, P200 and P10) Sterile filter pipette tips (1 ml, 200 and 10 al; Sigma-Aldrich, cat. nos. A3348, A3098 and A2473, respectively)
Sterile microcentrifuge tubes (1.5-ml size; Fisher Scientific, cat. no. 05-408-129)
Sterile 10-ml syringe without needle (Sigma, cat. no. Z248029)
Syringe filter, 0.2 μm (Sigma-Aldrich, cat. no. Z259969)
Stericup 0.2-μm filter unit (500 and 250 ml; Millipore, cat. nos. SCGVUO2RE SCGVU05RE, respectively)
U-bottom ultralow attachment plates, 96 well (Corning, cat. no. CLS7007)
Conical tubes, 15 ml (Fisher Scientific, cat. no. 05-527-90)
Tissue culture dish, 60 mm (Sigma-Aldrich, cat. no. CLS430589)
Spinner flask, 125 ml (Corning, cat. no. 4500-125)
Stir plate (2mag, bioMIXdrive and bioMIXcontrol)
Orbital shaker (IKA, cat. no. 0009019200)
Pipetboy (Integra Biosciences, cat. no. 155 000)
Serological pipettes, 10 ml (BD Falcon, cat. no. 357551)
Sterilized scissors
Water bath, 37° C. (Fisher Scientific, Isotemp water bath)
Stereomicroscope (Zeiss, Stemi 2000)
Inverted contrasting tissue culture microscope (Leica, DMIL)
Automated cell counter (Bio-Rad, TC10) or Hemocytometer
Benchtop centrifuge (Thermo Scientific, Heraeus Multifuge Is)
Weighing dishes (Sigma-Aldrich, cat. no. Z154873)
Scalpel blade (Sigma-Aldrich, cat. no. S2771)
Isopentane (2-methylbutane; Sigma-Aldrich, cat. no. M32631)
Dry ice
Low-temperature thermometer (Sigma-Aldrich, cat. no. Z257400)
Laboratory spatula (Sigma-Aldrich, cat. no. S3897)
Cryostat (Leica)
Cryomold
   Feeder-Independent hESC and hiPSC Lines:

The hESC and hiPSCs were cultured with mTeSR1 medium using standard practice in 5% CO₂ incubators set to 37° C. Cells were thawed into 60 mm dishes that were coated with geltrex (1:100 dilution dissolved in DMEM-F12). Thawed cells were passaged with 0.5 mM EDTA in sterile 1×PBS without calcium or magnesium from one 60 mm dish at 80% confluency into three 60 mm dishes.

### Reconstituting Growth Factors, Morphogens, and Small Molecules:

Initially, prepare a 10% (wt/vol) BSA/1×PBS solution by dissolving 1 g of BSA into 10 ml of sterile 1×PBS, which was stored in −80° C. for 1 year.

### Reconstitute 2 mg of CHIR in 2.52 mL of DMSO.

Reconstitute 100 mg of apo-transferrin in 10 mL of sterile water to obtain a 10 mg/mL solution.

Reconstitute 100 μg of Noggin in 1 mL of sterile 1×PBS+0.1% (wt/vol) final concentration BSA to obtain a 100 μg/mL solution. Reconstitute 100 μg of FGF8b in 1 mL of sterile PBS+0.1% (wt/vol) final concentration BSA to obtain a 100 μg/mL solution. Reconstitute 10 mg of SB431542 in 2.602 mL of DMSO to obtain a 10 mM solution.

Reconstitute 10 mg of ROCKi in 3.122 mL sterile water to obtain a 10 mM solution.

Reconstitute 100 μg of SDF1a in 1 mL of sterile PBS+0.1% (wt/vol) final concentration BSA to obtain a 100 μg/mL solution. Reconstitute 1 mg of BDNF in 10 mL of sterile 1×PBS+0.1% (wt/vol) final concentration BSA to obtain a 100 μg/mL solution. Reconstitute 50 mg of Heparin in 5 mL of sterile water to obtain a 10 mg/mL solution.

Reconstitute 250 mg of T3 (Triiodothyronine) into 12.8 mL of DMSO to get a 1:2000 dilution. Dissolve each of the 200 uL aliquots of DMSO in 200 uL of ammonia solution (4M in methanol) when ready to use at 1:1000 dilution.

Make 5 ml aliquots of N-2, 10 ml aliquots of B-27, 50 ml aliquots of knock-out serum replacement (KSR) and store it at −20° C. for up to 1 year. (The N-2 supplement formulation includes insulin, transferrin, progesterone, putrescine, and selenium; whereas the B-27 supplement formulation includes many of the same components as N-2 supplement, but also includes the thyroid hormone T3, fatty acids, and antioxidants, such as vitamin E and glutathione.) The components in N-2 Supplement are described by vendors such as ThermoFisher and include human transferrin, insulin recombinant full chain, progesterone, putrescine, and selenite. The components in B-27 serum-free are described by vendors such as ThermoFisher and include biotin, DL alpha tocopherol acetate, DL alpha-tocopherol, vitamin A (acetate), BSA (fatty acid free fraction V), catalase, human recombinant insulin, human transferrin, superoxide dismutase, corticosterone, D-galactose, ethanolamine HCl, glutathione (reduced), L-carnitine HCl, linoleic acid, linolenic acid, progesterone, putrescine 2HCl, sodium selenite, and T3 (triodo-l-thyronine). In some embodiments, KSR is not used, and fetal bovine serum is added. In some embodiments, the KSR includes Glycine, L-histidine, L-isoleucine, L-methionine, L-phenylalanine, L-proline, L-hydroxyproline, L-serine, L-threonine, L-tryptophan, L-tyrosine, L-valine, Thiamine, reduced glutathione, ascorbic acid 2-PO4, Ag+, A13+, Ba2+, Cd2+, Co2+, Cr3+, Ge4+, Se4+, Br−, I−, F−, Mn2+, Si4+, V5+, Mo6+, Ni2+, Rb+, Sn2+, Zr4+,Transferrin (iron-saturated), insulin, and lipid-rich albumin (AlbuMAX).

After thawing growth factors, morphogens, and small molecules, store them at 4 C for up to 1 week. Reconstituted solutions are stored at −20° C. for up to 6 months. Limit freeze thawing of all reagents. Once thawed, store at 4° C. for up to 1 week.

Aliquoting Matrigel: Thaw Matrigel on ice at 4° C. overnight. Pre-cool a sterile, 1 mL pipette tip box and ten microcentrifuge tubes at −20° C. for 15 min. In a sterile hood, quickly transfer 1 ml of Matrigel into each pre-cooled microcentrifuge tube. Store aliquots at −20° C. for up to 1 year. Avoid repeated freezing and thawing. Once thawed, place aliquot at 4 C and use as needed.

hiPSC and hESC medium: Add 100 ml of 5× supplement to ˜400 ml of mTeSR1 medium to form a complete medium that is used to conduct full media change on stem cells daily.

gfCDM+i medium: Weigh out 0.25 g of BSA and add it to a combined 1:1 ratio of 25 mL of IMDM with 25 mL of Ham's F-12, (fatty acid free+globulin free), leading to a final concentration of 5 mg/ml BSA. Let that dissolved in the 4° C. for about 1 hour prior to adding apo-transferrin (final concentration of 15 ug/mL), insulin (final concentration of 7 ug/mL), 1% (vol/vol) chemically-defined lipid concentrate (CDLC; final concentration 2 mM), mono-thioglycerol (final concentration of 0.1 mM), and 1% (vol/vol) Pen/Strep. Once all components are added, filter sterilize the media. Thus, Patterning Media for days 0-6 of the protocol (about 50 ml) is obtained.

CerDM1 medium: Combine DMEM-F12 with KSR (final KSR volume percentage is 20% (vol/vol)), 1% (vol/vol) GlutaMAX supplement (final concentration 2 mM), apo-transferrin (final concentration of 15 ug/mL), insulin (final concentration of 7 ug/mL), beta-mercaptoethanol (BME) (final concentration of 0.1 mM), Pen/Strep (final volume percentage: 1% (vol/vol)). Thus, Patterning Media (for days 6-16 of the protocol) is obtained.

CerDM2 medium: Combine DMEM-F12 with N-2 (final N-2 volume percentage is 1% (vol/vol)), 2% (vol/vol) B27+vitA, 1% (vol/vol) GlutaMAX (final concentration 2 mM), P/S (final concentration 1% (vol/vol)). Thus, Induction Media (for Days 16-30 of the protocol) is obtained.

CerDM3 medium: Combine 1:1 ratio of DMEM-F12 and Neurobasal with 1% (vol/vol) N2 (final volume percentage of N-2 is 1%), 2% (vol/vol) B27+vitA, 1% (vol/vol) GlutaMAX (final concentration 2 mM), 1% (vol/vol) CDLC (final volume percentage 1%), Heparin (final concentration of 5 ug/mL), 0.1% (vol/vol) Fungizone. T3 is added right before use at a final concentration of 0.5 ng/mL, Matrigel is added right before use at a final volume percentage of 1%, and SDF1a is added right before use at a final concentration of 100 ng/mL. Thus, Maintenance Media (for Days 30-60 of the protocol) is obtained.

Note: gfCDM+i, CerDM1, CerDM2, and CerDM3 can be stored for up to 2 weeks at 4° C. after making it.

CerDM4 medium: Same as CerDM3 media but with BDNF (target concentration is 14 ng/mL; added right before use). Thus, Maintenance Media for Days 60+ is obtained.

BrainPhys medium: Maintenance Media prior to Ephys analysis (50 mL) includes BDNF (target concentration 14 ng/mL; added right before use) and T3 (target concentration 0.5 ng/mL; added right before use). K-gluconate internal solution (Maintenance Media prior to Ephys analysis): K-D-gluconate (2873 mg in 100 ml), KCl (93.19 mg in 100 ml), KOH-EGTA (7.6 mg in 100 ml), KOH-Hepes acid (238.3 mg in 100 ml), NaCl (46.7 mg in 100 ml), Mg2ATP (101.44 mg in 100 ml), Na3GTP (15.69 in 100 ml), and KOH added to adjust pH to 7.3. In some embodiments, this is prepared in water, distilled water, or milliQ water.

### Toxins and Drugs Preparation:

Reconstitute 1 mg of NBQX in 496 ul of Milli-Q water to obtain a 5 mM solution. Use at final concentration of 5 uM. Reconstitute 50 mg of D-AP5 in 101.45 ul of Milli-Q water to obtain a 50 mM solution. Use at final concentration of 50 uM. Reconstitute 10 mg of NMDA in 6.8 mL of Milli-Q water to obtain a 10 mM solution. Use at final concentration of 3-100 uM. Reconstitute 301.295 mg of Picrotoxin in 5 mL of DMSO to obtain a 100 mM solution. Use at final concentration of 100 uM. Reconstitute 1 mg of Tetrodotoxin in 3.13 mLl of Milli-Q water to obtain a 1 mM solution. Use at final concentration of 1 uM.

### Papain Dissociation Preparation:

Prepare a 10% (wt/vol) BSA/1×PBS solution by dissolving 1 g of BSA into 10 ml of sterile 1×PBS, which was stored in −80° C. for 1 year. Use this to further dilute it to a 0.04% (wt/vol) BSA/1×PBS solution in order to create final cell suspension after dissociation of organoids. Dissolve one bottle of lyophilized papain with 5 mL of Earle's media. Dissolve one bottle of DNase with 500 uL of Earle's media. Dissolve one bottle of Inhibitor with 32 mL of Earle's media.

### Spinning Bioreactors:

Place and install a CO₂ resistant low-speed stir plate into the tissue culture incubator by sterilizing it with 70% ethanol. Place the shelves of the incubator in a way that allows easy placement and removal of the bioreactors on the stir plate. Exit the power cord out the back of the incubator and through a stopper to prevent excess CO₂ leakage out of the incubator. Tape the chords to hold them in place.

### Orbital Shaker

Sterilize the rubber mat platform with 70% ethanol prior to installing it onto the orbital shaker. Place and install a CO₂ resistant orbital shaker into the tissue culture incubator by sterilizing it with 70% ethanol. Move the shelves to allow ample vertical space to stack the 10 cm dishes as well as to easily place/remove the 10 cm dishes. Place the control box on the side of the incubator and exit the power cord and control cord out the back of the incubator and through a stopper to prevent excess C02 leakage out of the incubator. Tape the chords to hold them in place.

Note: Make sure that the 10 cm dishes have ridges on the bottom of the plate that allow them to lock into one another or else stacking plates is not possible. A standard tissue culture incubator will allow for the placement of either an orbital shaker or a spinning bioreactor, but not both.

### 2-Photon Calcium Imaging

Set wavelength of 2-photon to 880 nm with emission spectrum between 400-550 nm. Set time lapse image acquisition for 10 minutes at acquisition rate 10 Hz. Set image size to 512×512 pixels. Before the start of recordings, set the stage to 37.5 Celsius. Place pipettes and tips at the microscope station. Safely position bleach in a glass bottle for the disposal of tips contaminated with drugs.

### Patch Rig

Thaw the internal solution and keep it on ice for the remainder of the experiment. Add 2 mL of BrainPhys Optimized Medium into the recording chamber. Prepare 3-5 MOhm patch pipettes from glass capillaries. Add 3 ul of internal solution in each pipette. Test the resistance by mounting the pipette on the electrophysiology rig holder and submerge into the recording chamber medium. Before the start of recordings, set the stage to 37.5 Celsius.

### Procedures

### Making EBs: Timing 1-2 hrs (Day 0)

Grow the hPSCs (or iPSCs) in one 60 mm dish coated with Geltrex (Geltrex matrix is a basement membrane extract that contains laminin, collagen IV, entactin, and heparin sulfate proteoglycans) until the plate is 70-80% confluent. This whole plate will be used to start the differentiation. Typically, one confluent plate can be used to seed about three 96-well plates.

Note: The proper morphology of the stem cell colonies is essential for a successful differentiation. Therefore, make sure that the colonies do not have any signs of differentiation.

Take feeder-free hPSCs being cultured and feed the plate 1 hour prior to starting differentiation with fresh mTeSR media.

Wash the plate of hPSCs by removing mTeSR media and add 2 ml of 1×PBS.

After swirling the plate a few times, remove the 1×PBS and add 2 ml of ACCUTASE (an enzyme mixture with proteolytic and collagenolytic enzyme activity) to the plate to initiate dissociation of the cells and place plate in the incubator set to 37° C. for 3-5 minutes depending on the confluency of the plate and the density of the cell colonies.

Note: Make sure to not keep the ACCUTASE on the cells for longer than 7 minutes as this causes damage to the cells and does not yield a good starting EB for differentiation.

Quench the ACCUTASE with at least 4 mL of DMEM/F12 and transfer to a 15 ml conical tube to triturate the cell clumps ˜10 times to break them up into a single cell suspension.

Using 10 μl of Trypan blue at a 1:1 ratio with 10 μl of the cells in DMEM/F12 suspension, count the live cells on a hemocytometer while the cells spin in a centrifuge at 200×g for 5 minutes at room temperature.

Remove the supernatant from the cell pellet and resuspend the pellet in 1 ml of DMEM/F12. Transfer 600,000 cells from the resuspended pellet into a 15 mL conical tube containing 10 mL of gfCDM+i media containing 10 μM TFGBi (TGF-β Small Molecule Inhibitor, SB431542), 1.7 μM CHIR, 50 ng/mL of Noggin, and 20 μM of ROCKi.

Pour this cell suspension into a reservoir and add 100 ul to each well of one 96-well V-bottom plate using a multichannel P200 pipette. 24 hours later you should observe a small embryoid body (EB) with defined borders under a microscope. Maintain these organoids within a tissue culture incubator set at 37° C. and 5% CO₂.

Note there can be a few dead cells around the EB, but if there is excessive cell death, the differentiation must be terminated.

### Feeding EBs and Initiation of Germ Layer Differentiation: Timing 4 Days (Day 0-4)

Day 2: prepare gfCDM+i containing 10 μM TGFBi, 1.7 μM CHIR, 150 μg/mL Noggin, and 20 μM ROCKi. Remove 40 μL of media from each well without disturbing the cells at the bottom of the well and add 50p of the freshly prepared gfCDM+i.

Day 4: prepare gfCDM+i containing 10 μM TGFBi, 1.7 μM CHIR, 100 μg/mL Noggin, 200 ng/mL FGF8b. Remove 40 μl of media from each well and add 50p of this freshly prepared gfCDM+i.

### Induction of Isthmic Organizing Region: Timing 11 Days (Day 4-16)

Day 6: prepare CerDM1 containing 10 μM TGFBi, 1.7 μM CHIR, 100 ng/mL Noggin, and 100 ng/mL FGF8b. Remove 40 ul of media from each well and add 50 uL of this freshly prepared CerDM1.

Day 8: prepare double the volume of CerDM1 containing 10 μM TGFBi, 1.7 μM CHIR, 100 ng/mL Noggin, and 300 ng/mL FGF8b. Remove 40 μl of media from each well and add 100 μL of this freshly prepared CerDM1, which brings the total volume up to 150 μL.

Note: Be sure to increase the concentration of FGF8b from 100 ng/mL to 300 ng/mL from Day 6 to Day 8.

Day 10: repeat the previous step of Day 8, but this time remove 80 uL of media and add 100 uL of freshly prepared CerDM1.

Day 12: simply conduct a half media change by removing 70 uL of media and replacing it with CerDM1 that does not contain any drugs or morphogens.

Day 14: repeat half media change as was done on Day 12. By this day, a bit of cell debris may be noticed around the edge of the organoid in which case a “wash step” may be conducted by disturbing the pellet by pipetting the media in the well up and down prior to removing it and replacing it with fresh CerDM1 to remove as much debris as possible.

### Shaking Culture of Expanding Neuroepithelial Buds: Timing 13 Days (Day 16-29)

On Day 16 of differentiation, transfer the organoids from the 96-well V-bottom plates equally into two 10 cm dishes.

Prepare sterile scissors by spraying the open scissors with 70% ethanol and leaving it open to dry in the culture hood. Then cut the edge of a 200 uL pipette tip to create a wide-bore pipette tip.

Transfer the organoids into a 10 cm plate containing one fifth of old media (3 mL) from the 96-well V-bottom plate and four fifths (12 mL) of CerDM2 media (bringing the total to 15 mL in the 10 cm plate).

Noted: Do not add more than half a 96 well plate full of organoids (48 organoids) into one 10 cm dish as this may affect the overall health of the organoids over the long term.

Culture the organoids on a CO2 resistant orbital shaker placed inside of an incubator set at 37° C. and 5% CO₂. Set the shaker for 70 r.p.m. Make sure to use a low speed, CO2 resistant shaker intended for tissue culture use inside of an incubator. A standard orbital shaker may overheat and be damaged by humidity.

Conduct a full media change by removing the media within the 10 cm plate and replacing it with fresh 15 mL of CerDM2 every 3 days.

Growth and Maturation of Cerebellar Tissue (from Day 30 to 1 Year): Timing Over 1 Year

Conduct a full media change by removing the media within the 10 cm plate and replacing it with fresh 15 mL of CerDM3 starting at Day 30 with of T3 added to a final concentration of 0.5 ng/mL directly to the media prior to feeding and add BDNF to a final concentration of 14 ng/mL starting from Day 60.

If you intend to create a stratified layered organization within the organoids, add 100 ng/mL of SDF1a and a 1:100 dilution (1%) of growth factor reduced matrigel dissolved directly into the media from Day 30-60. The SDF1a and growth factor reduced matrigel are not necessary to obtain the cell types of interest. Add these components to the 10 cm plate every 3 days in addition to the T3 when conducting the full media change until Day 60 to obtain the organized layering. Alternatively a 125 mL flask can be used on a low-speed stir plate instead of a 10 cm dish on orbital shaker

Note: Add Matrigel, T3, SDF1a, or BDNF into CerDM3 on the same day that you intend to use the media. The base CerDM3 media can be stored for up to 2 weeks in 4° C. gfCDM1+i, CerDM1, and CerDM2 can also be stored at 4° C. for up to 4 weeks after preparing.

Starting from Day 30 onward, the organoids can be subject to various procedures to be analyzed at various time points. These may include cryosectioning and immunostaining the organoids (option A), dissociation into single cell suspension for further analysis at a single cell resolution (option B), calcium imaging (option C), patch-clamp recordings on whole organoids (option D).

### Option A: Cryosectioning and Immunostaining Organoids: Timing 2-3 d

1. Transfer organoids into a 1.5 mL conical tube using a cut 200 ul tip (or 1000 ul tip organoids are over 60 days old).
Note: Make sure to use sterile scissors to cut the tip.
2. Remove as much of the media as possible without damaging the organoids and completely submerge the organoids in at least 500 uL of 4% PFA (wt/vol) and let it stay at room temperature for 30 min. Gently remove the PFA, dispose of it properly, and replace it with 1 mL of 1×PBS. Let it sit at room temp for 5 min and repeat this 1×PBS wash twice more.
3. Remove the last 1×PBS wash and replace it with 500 uL of 30% sucrose (wt/vol) and let it sit overnight at 4 C until the organoids submerge to the bottom of the tube.
Note: Make sure the organoid is floating on the surface when placed in sucrose. If the organoid is still floating after overnight incubation at 4 C, attempt to push the organoid down with a 200 uL tip to see if it starts to sink. If the organoids sink immediately after sucrose is added to the tube or does not sink after overnight incubation, terminate the experiment.
4. The next day, transfer the organoids into a cryomold and remove all of the sucrose by pipetting it off.
5. As the organoids sit on the bottom of the cryomold, add Tissue-Tek O.C.T. into the mold and cover the organoids completely.
Note: Do not add too many organoids as this makes sectioning and collecting intact samples difficult. Make sure the organoids have some space between one another and are away from the walls of the mold.
6. Place the embedded organoids on dry ice directly until the O.C.T. turns completely white.
Note: You may store these molds at −80° C. for over 1 year until they are ready to section.
7. Transfer the organoid blocks to a cryostat and let it equilibrate to the chamber temperature of −20 C for 15 min prior to sectioning.
8. Section the organoids at 14-20 um and collect them onto glass slides.
9. Stain organoids using a standard immuno technique. Antibodies used are found in Table 10.
10. If you wish to perform whole organoid staining, increase the incubation times of primary and secondary antibodies to 48 hours at 4 C on a nutator.
   Option B: Dissociating Organoids into Single Cells: Timing 2-3 hr
1. Transfer one organoid from the spinner flask or 10 cm dish into a 60 mm cell culture dish containing 5 mL of prewarmed Worthington papain solution 500 μL of Worthington DNase solution.
2. Mince the organoid into small pieces with a razor blade.
3. Place the 60 mm dish that contains the tissue onto an orbital shaker in a cell culture incubator, and incubate the chopped up organoid on the shaker with a speed set at 70 rpm for 30 minutes at 37° C.
4. Triturate gently using a 1 mL pipette tip several times to break up the minced pieces.
5. Incubate for an additional 5-10 min at 37° C.
6. Triturate with 10 mL serological pipette ˜10 times. Allow any pieces of undissociated tissue remaining after trituration to settle to the bottom of the tube.
7. Transfer cell suspension to a 15 mL conical tube and let the debris and undissociated tissue settle to the bottom of the tube after 1-3 min.
8. Transfer cell suspension (not containing settled debris) to a new 15 mL conical tube already containing 5 mL of Earle's medium+3 mL of Inhibitor Solution.
9. Pellet the cells by centrifugation for 7 min at 300 g.
10. Remove supernatant and resuspend the cells in 500 uL to 1 mL of 0.04% BSA/1×PBS.
11. Filter cells through a 0.22 um filter basket.
12. Count cells using trypan blue.
13. Resuspend cells to a target concentration of 1000 cells/uL
14. Place cells on ice until ready for further experimentation.

### Option C: Calcium Imaging of Whole Organoids: Timing 7-10 d

1. Place 3 organoids in one well of a 24 low attachment well plate with 150 ul of CerDM3 media.
Note: Add in the well enough media to cover the organoids. No excess media should be added to the organoid as this will impact the success of the viral transfection.
2. Add 1.5 ul of pAAV-CAG-SomaGCaMP6f2 by tilting the plate, letting the organoids sink on the bottom of the well and releasing the volume directly on top of the organoids without contacting them with the pipette tip.
If a calcium imaging experiment is performed together with optogenetic stimulation, add 1.5 ul of L7-eOPN3 Lentivirus immediately after SomaGCaMP6f2 infection. Note: Carefully rotate the dish to prevent organoids from coming into contact with each other and to avoid the fusion of organoids. Place the plate in the incubator on static conditions.
3. After 24 hrs perform a full media change with 500 ul of the same CerDM4 media used for viral infection.
4. After 48 hrs perform half media change with BrainPhys Medium.
5. After 72 hr transfer the organoid to a 6-well low attachment plate with 2 mL of BrainPhys Medium. Perform half-media change with BrainPhys Medium every 2 days.
6. After 5 days from viral transfection, routinely check the appearance of GreenFluorescentProtein (GFP) for SomaGCaMP6f2.
7. Prepare organoids for calcium imaging by selecting the ones with the appearance of a strong cell specific fluorescent signal. Add 5 ul of Culturex to a Glass Bottom Microwell Dish Promptly positioning the organoid over it with a 50 ul media droplet. Place in a static condition in the incubator.
8. After 30 minutes, add 2 mL of BrainPhys Optimized Medium and bring the organoid to the 2-photon microscope. Note: Transfer the organoid with minimal motion, using a styrofoam box, to prevent fluctuations in room temperature.
9. Perform time-lapse recording of the organoids. Note: Be aware that the laser generates heat. Therefore, closely monitor the stage temperature to ensure it does not exceed 37.5 degrees Celsius.
10. For optogenetic stimulation of L7-eOPN3, perform a 525 nm LED laser stimulation for 500 ms at maximal intensity. After stimulation, time-lapse record calcium responded as in step 9.
11. Drugs can be added to the petri dish to modulate the organoid activity. Specifically, the AMPA antagonist NBQX, the NMDA antagonist D-AP5, the GABA antagonist Picrotoxin, the NMDA agonist NMDA and the Na+ channel blocker Tetrodotoxin. Note: Pause the recording to avoid eye exposure to laser and carefully add the drug of interest in the media by using gloves and tossing the used pipettes in decontaminating bleach. Wait 60 seconds to allow drug diffusion and start recording again.

OPTOGENETICS (OPTOGENETIC MODULATION OF PURKINJE CELL FUNCTIONALITY; Timing: 7-10 d): additional reagent/material/equipment includes L7-eOPN3 Lentivirus and 525 nm LED laser. Procedures include: 1) Perform lentiviral infection with L7-eOPN3 Lentivirus as in step 2-6 in “Option C”. Start recordings upon the appearance of strong Red Fluorescent Protein (RFP) for opsins expression. 2) Position the organoids in the imaging chamber as performed in step 7 and 8. 3) Place the LED laser in a position where it directs towards the organoid, covering the exact area where imaging will take place. 4) Perform a 525 nm LED laser stimulation for 500 ms at maximal intensity. Perform time-lapse recording of the organoids.
Note: Be aware that the laser generates heat. Therefore, closely monitor the stage temperature to ensure it does not exceed 37.5 degrees Celsius.

### Option D: Patch-Clamp Recording of Whole Organoids: Timing 7-10 d

1. Perform a viral infection with AAV8.L7-6.eGFP.WPRE.hBG to label Purkinje Neurons. Follow step 1 to 6 of “Option C” for viral infection procedure.
2. Prepare organoids for Patch-clamp recordings by choosing those exhibiting a robust, cell-specific fluorescent signal.
3. Transfer a single organoid in the recording chamber and let the organoid sink at the bottom of the recording chamber.
4. Focus by brightfield on the edge of the organoid and use the green channel for selecting the cell to patch.
5. Reach the seal with the patch pipette and perform current clamp recording after break-in. (See Atamian et al for different recordings made with patch clamp).
6. Drugs can be added to the recording chamber to modulate the organoid activity. Note: Pause the recording to avoid noise signals and carefully add the drug of interest in the media by using gloves and tossing the used pipettes in decontaminating bleach. Wait 60 seconds to allow drug diffusion and start recording again.

Overall, the time involved for this protocol:
Step 1-8, Making EBs: 1-2 hr
Step 9-10, Feeding EBs and Initiation of germ layer differentiation: 4 days
Step 11-15, Induction of Isthmic organizing region: 11 days
Step 16-18, Shaking culture of expanding neuroepithelium: 13 days
Step 19-21, Growth and maturation of cerebellar tissue: >1 year

TABLE 3
Exemplary trouble-shooting details.

| Problem | Possible Reason | Solution |
|---|---|---|
| Small EBs, no | Seeded too little cells | Check accuracy of cell counting. |
| expansion | Unhealthy hPSCs | Robust stem cells should still be alive in Trypan |
| | | blue solution after 30 min. If most/all cells are |
| | | blue, then perform dissociation faster and more |
| | | gently. |
| | Density of hPSCs | Check pluripotency by morphology of stem |
| | was too high prior to | cells and stain for pluripotent markers. |
| | dissociation | |
| Disintegrating | Too many cells | Check accuracy of cell counting. |
| organoids in 96-well | plated in one well | |
| V-bottom plates and | | |
| media is very yellow. | | |
| Outgrowths and buds | Generation of other | Check pluripotency by morphology of |
| forming from the | germ layers | stem cells and stain for pluripotent markers. |
| organoid | | Make sure dual SMAD inhibitors have been |
| | | stored properly, not undergone too many freeze |
| | | thaws, and added at the correct concentration. |
| Disintegrating | Unhealthy hPSCs | Check pluripotency by morphology of stem |
| organoids after transfer | | cells and stain for pluripotent markers. |
| | | Make sure CerDM2 media is fresh. |
| | Rough transfer | Make sure the tip cut off of the pipette tip to |
| | step/serrated pipette | create a wide bore is smooth and without jagged |
| | tip | edges. |
| Media yellow/acidic | Too many organoids | Feed the plates with 12-15 ml of media between |
| prior to next feeding | in one plate | Day 16-30 |
| | | Feed the organoids in the bioreactor with a |
| | | minimum of 50 mL |
| | | If not adjusted for, the organoids will be more |
| | | sensitive overtime and disintegrate easily. |
| Low/no | Low viral titer | Make sure viral titers are sufficient, if not then |
| GcAMP/opsins viral | | add more of the virus to the initial incubation. |
| labeling after | Not enough | Wait at least 24 hours prior to conducting full |
| transfection | incubation time | media change after initial inoculation of |
| | | organoids with virus |
| | Disintegrating | Examine under brightfield microscopy to ensure |
| | organoids | that organoids exhibit a healthy morphology with |
| | | smooth edges. Any organoid displaying |
| | | disintegrating edges or cell death following viral |
| | | transfection should be excluded. |
| | Diffuse fluorescent | The GFP and RFP signals should manifest as |
| | signal | sharp and contained within the cell soma. While |
| | | a diffuse signal may be acceptable within the |
| | | initial days post-infection, if no cell-specific |
| | | fluorescent signal is observed by the second |
| | | week after infection, it is necessary to infect new |
| | | organoids. |
| Equipment setup Patch | Noise signals in the | Volume of media in the recording chamber |
| Clamp | recordings | depends on the type and size of chamber used. |
| | | Adjust volume so that is enough for the organoid |
| | | and the reference electrode to be submerged while |
| | | avoiding over floating and spillage of media as |
| | | this can cause noise signals in the recordings and |
| | | damaging equipment. |
| | Difficulty in creating | amount of internal solution should be adjusted |
| | a stable patch | depending on the length and shape of patch |
| | | pipettes. It is important to have enough internal |
| | | solution to reach the recording electrode, however |
| | | large volumes of internal solution should be |
| | | avoided for ensuring optimal seal |
| | Wrong resistance on | find proper resistance of patch pipette before |
| | Patch Pipettes | the beginning of the recordings. If resistance is |
| | | not within 3-5 MOhm check under brightfield |
| | | image that the pipette has a clear internal |
| | | solution without debris. If debris or small |
| | | particles are visible, filter the solution with a |
| | | 0.22-μm PES Filter System. If no debris are |
| | | present, change parameters on puller (according |
| | | to provider) to adjust up to the right values. |
| Patch-clamp | Cells are difficult to | select gfp cells on the edge of the organoid with |
| recordings | patch | a clear visible soma coming out from the edge. |
| | | Internal cells will not be accessible for patch |
| | | clamp recordings. |
| | Difficulty in | leave the patch in attached mode for up to 10 |
| | reaching a seal | minutes and apply negative pressure for few |
| | | times to help braking in the membrane |

This protocol aims to generate mature cerebellar organoids that contain both inhibitory and excitatory populations of the developing cerebellum. This method is superior to previous techniques that rely on the use of co-culturing cells with mice granule cells and glia to achieve a high level of maturation within the Purkinje Cells. The hCerOs have the ability to model long term growth and maturation of the nascent cerebellar cell types allowing for increased level of network activity and connection between neuronal cell types further enhancing the system and making it more physiologically relevant.

Across cell lines, there is a consistent way in which these organoids grow within the first few days. They start to show signs of neuroectodermal differentiation with the appearance of rosette-like structures within the organoid around day 6 of differentiation. If the cells do not aggregate properly, the differentiation will yield suboptimal results. Once transferred to 10 cm dishes at day 16, the organoids may display slightly different morphologies by either appearing slightly smaller and more translucent, large/smooth and more opaque, or bulbs of growing neuroepithelium around the organoid giving it a bumpy shape. These morphologies are typical of cerebellar organoids deriving from various cell lines, however, it is essential to culture these organoids out to day 30 in order to confirm the presence of both progenitor markers.

As the organoids are cultured out to day 30, the presence of translucent rosette-like neuroepithelial structure may appear, which is indicative of a successful differentiation. However, modifying the protocol by maintaining the organoids in gfCDM instead of switching them to CerDM1 at day 6 changes the way these organoids develop in terms of their continuous, translucent neuroepithelial morphology and their cell type composition. It is important to note that the typical morphology of an optically translucent organoid to indicate a successful protocol may not apply to all cerebellar organoids. This is because some cell lines may not be as successful in forming rosette-like structures given that consistent WNT administration has been shown to disrupt ventricle formation. If the organoids seem large and opaque, it is important to first section the organoid to determine if there are smaller rosettes forming within the organoid prior to terminating the experiment. Additionally, the presence of translucent rosette structures alone will not be a good indicator of a successful cerebellar differentiation as these tighter and more uniform translucent rosette structures are more likely cortical organoids derived from suboptimal patterning.

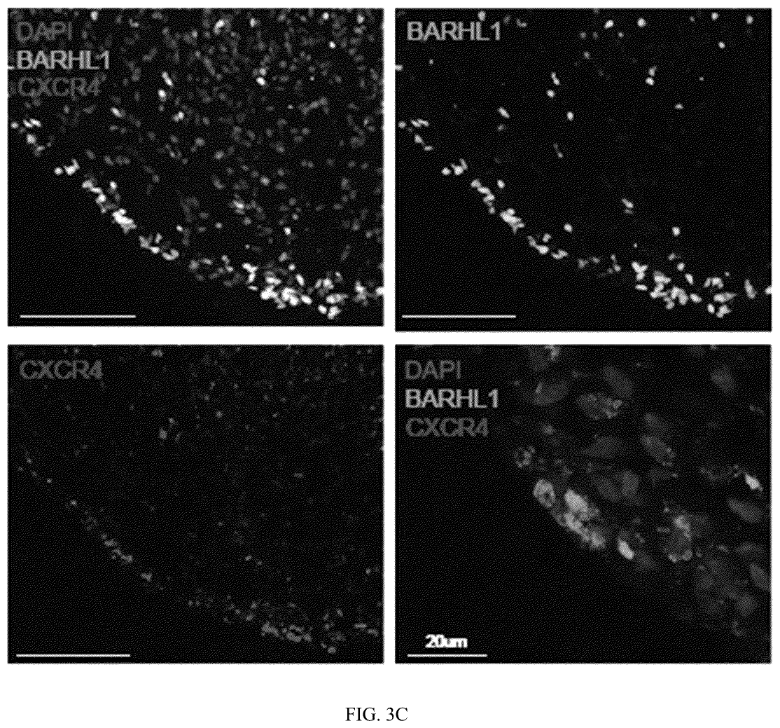

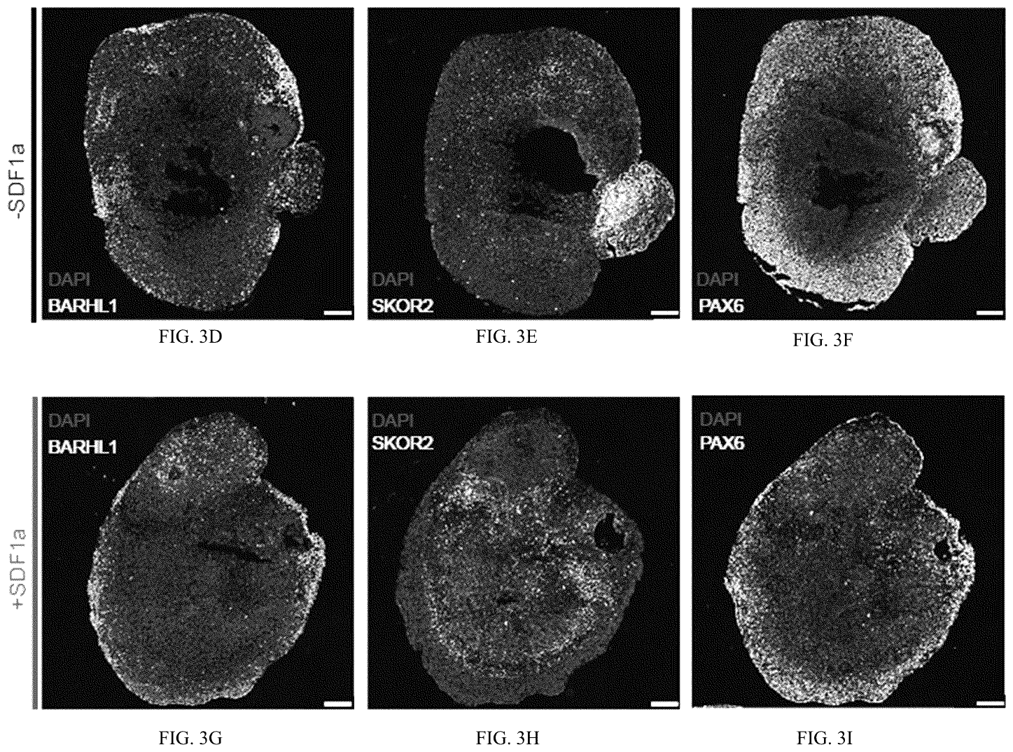

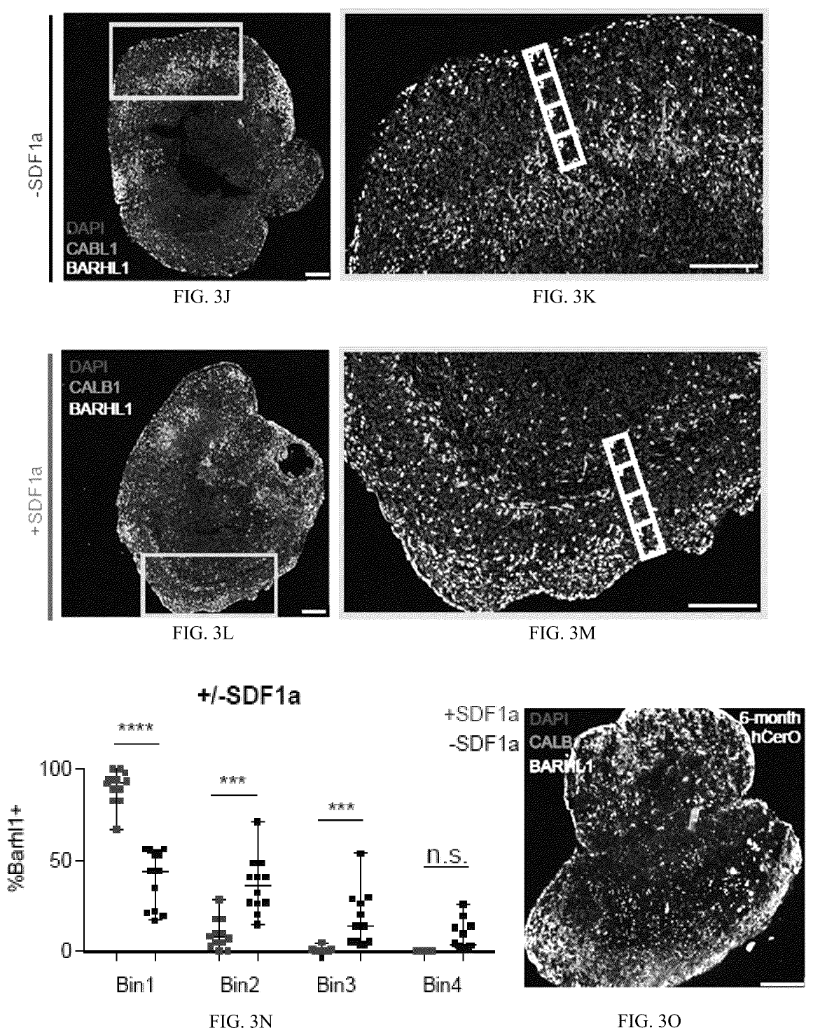

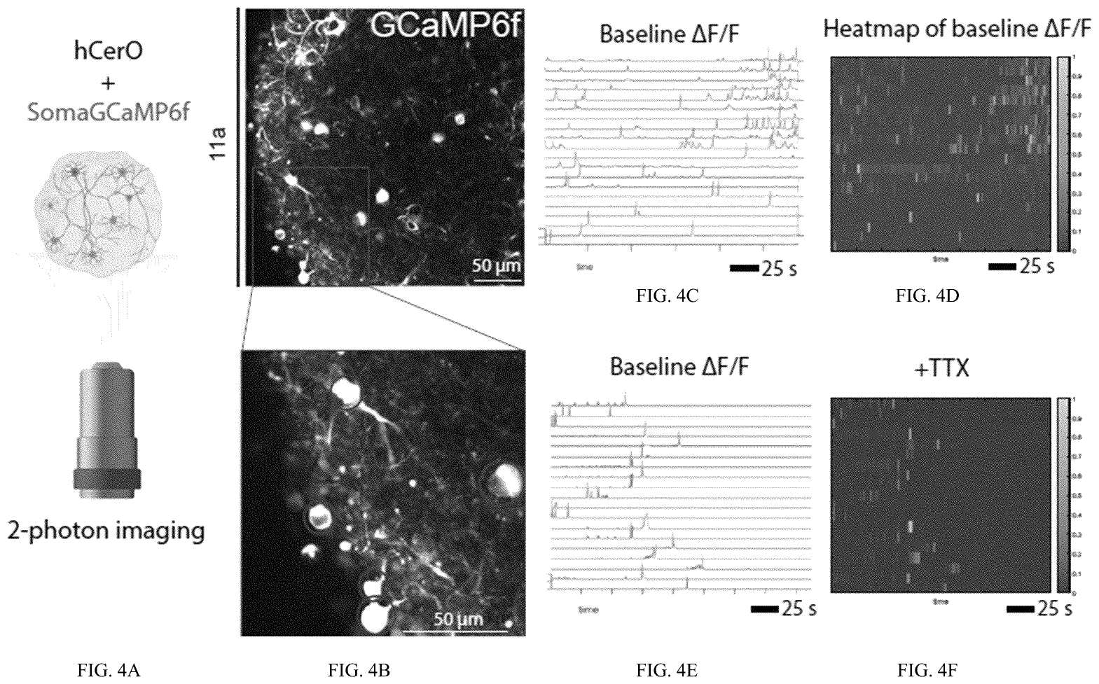

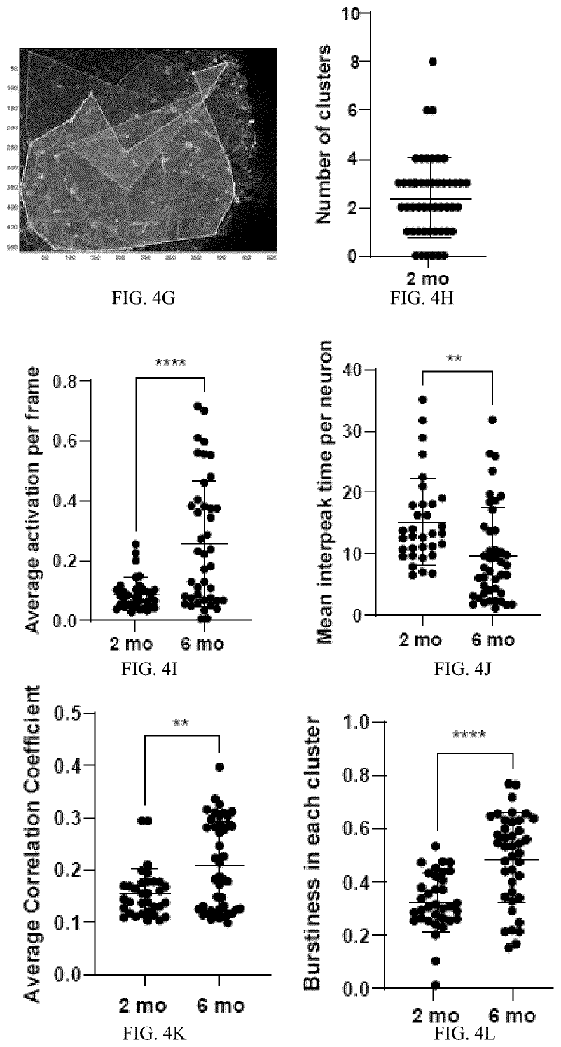

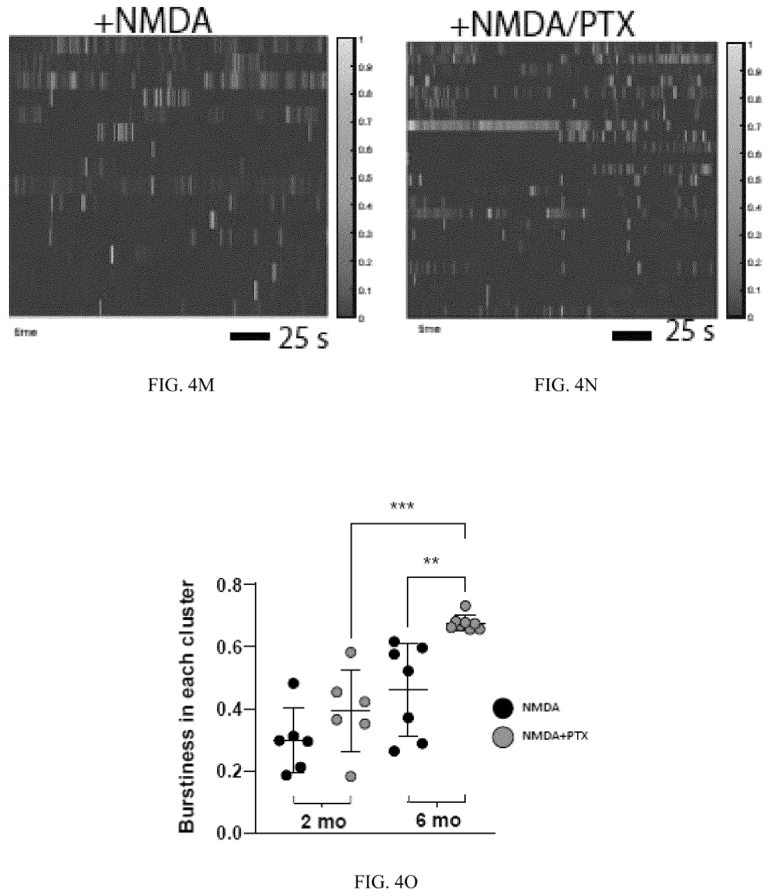

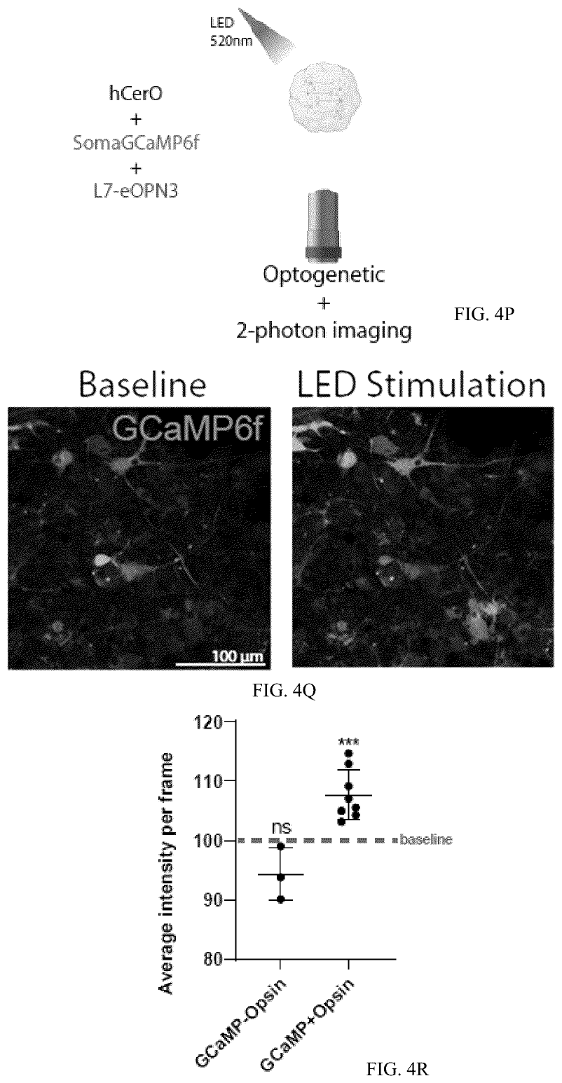

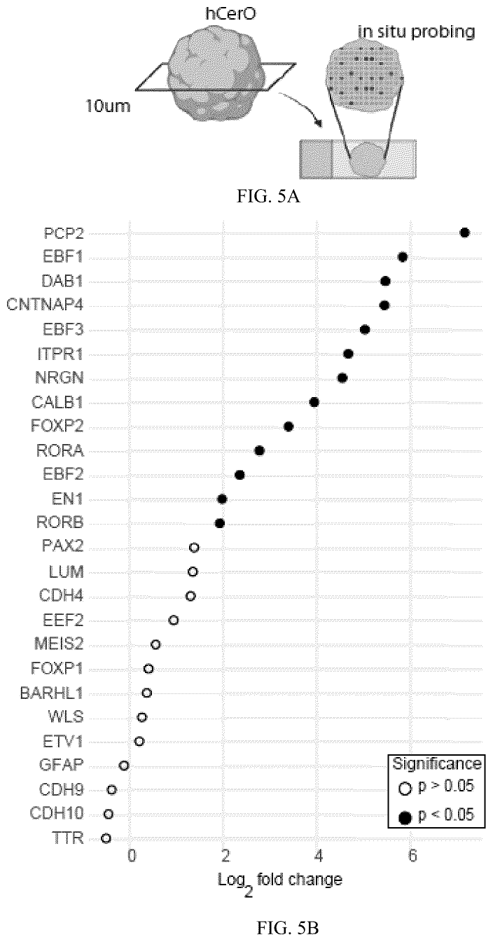

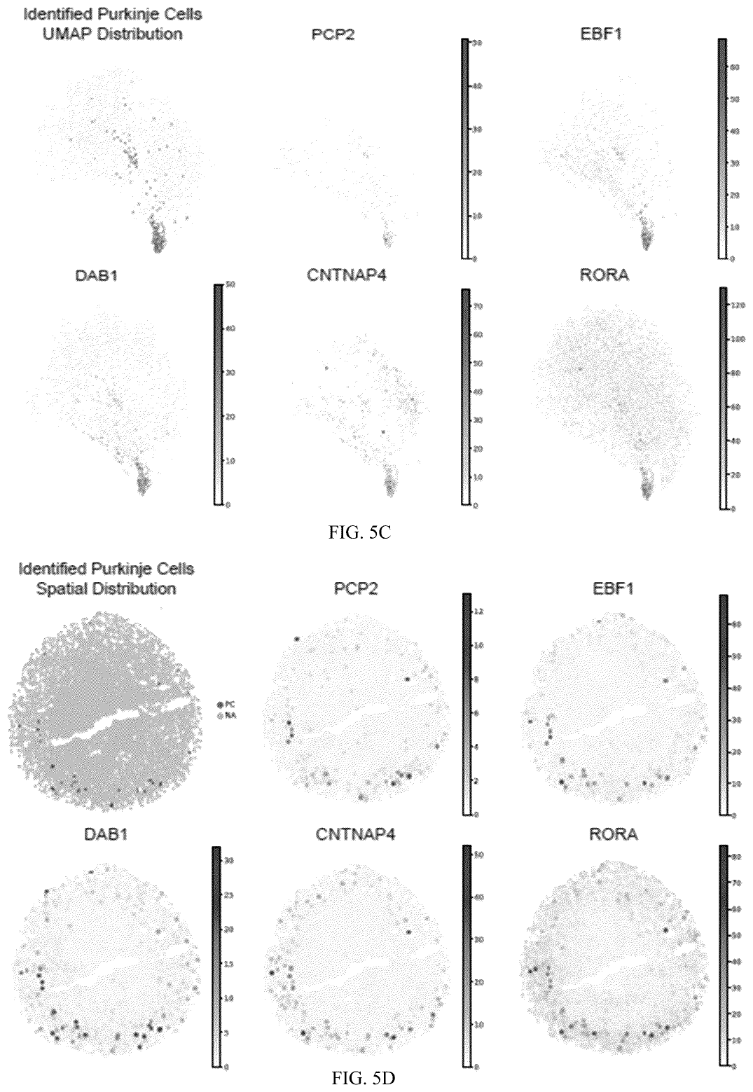

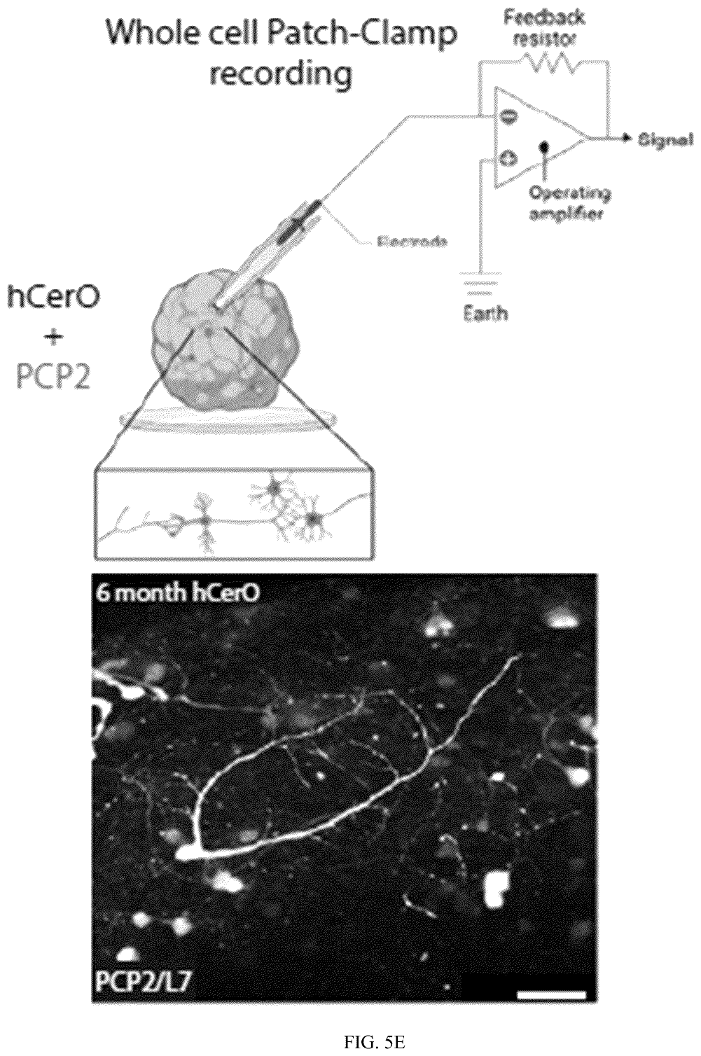

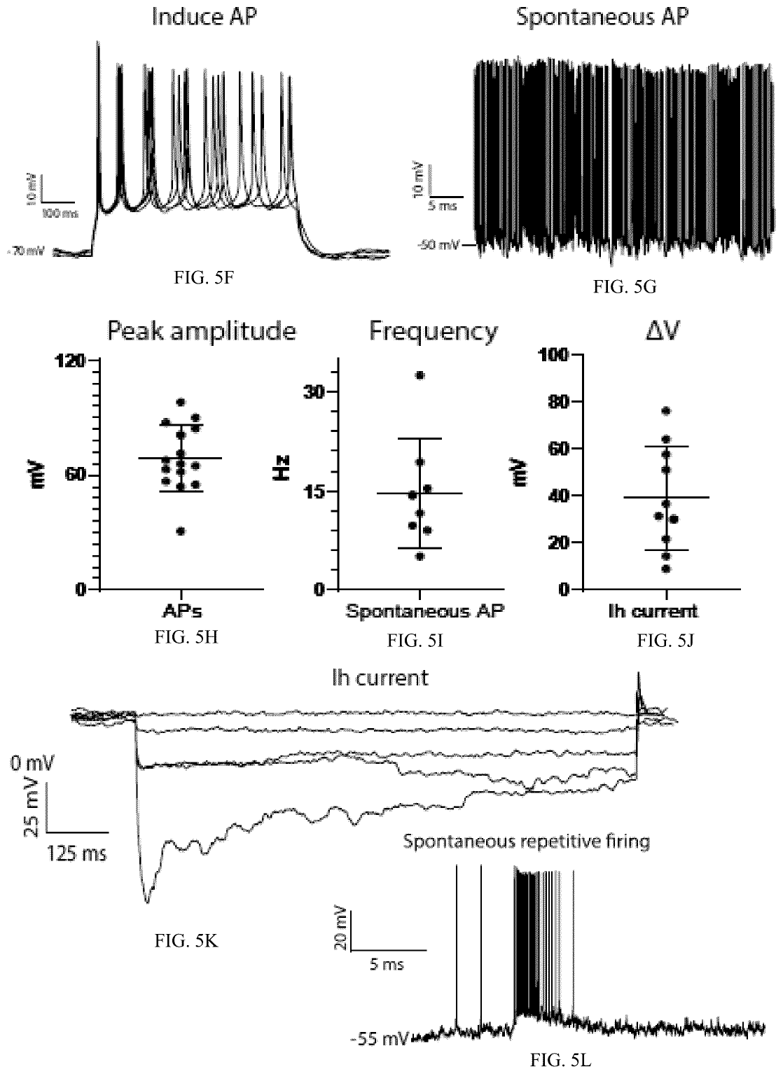

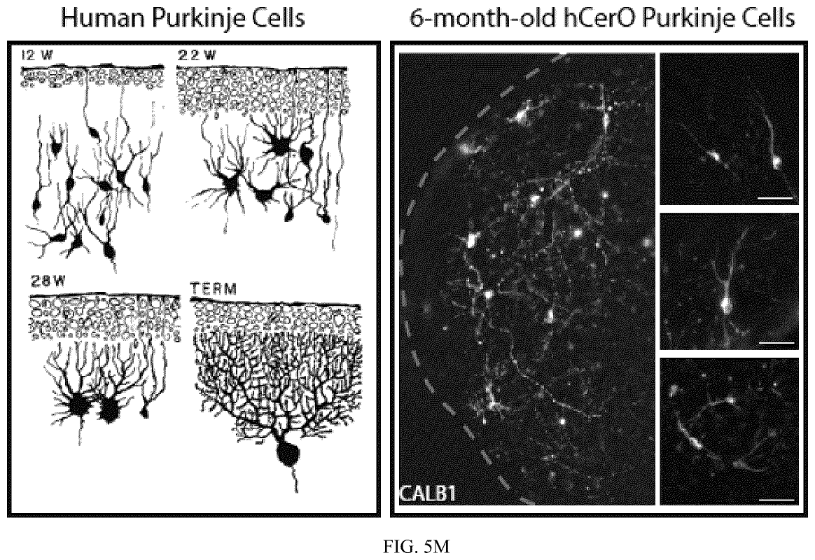

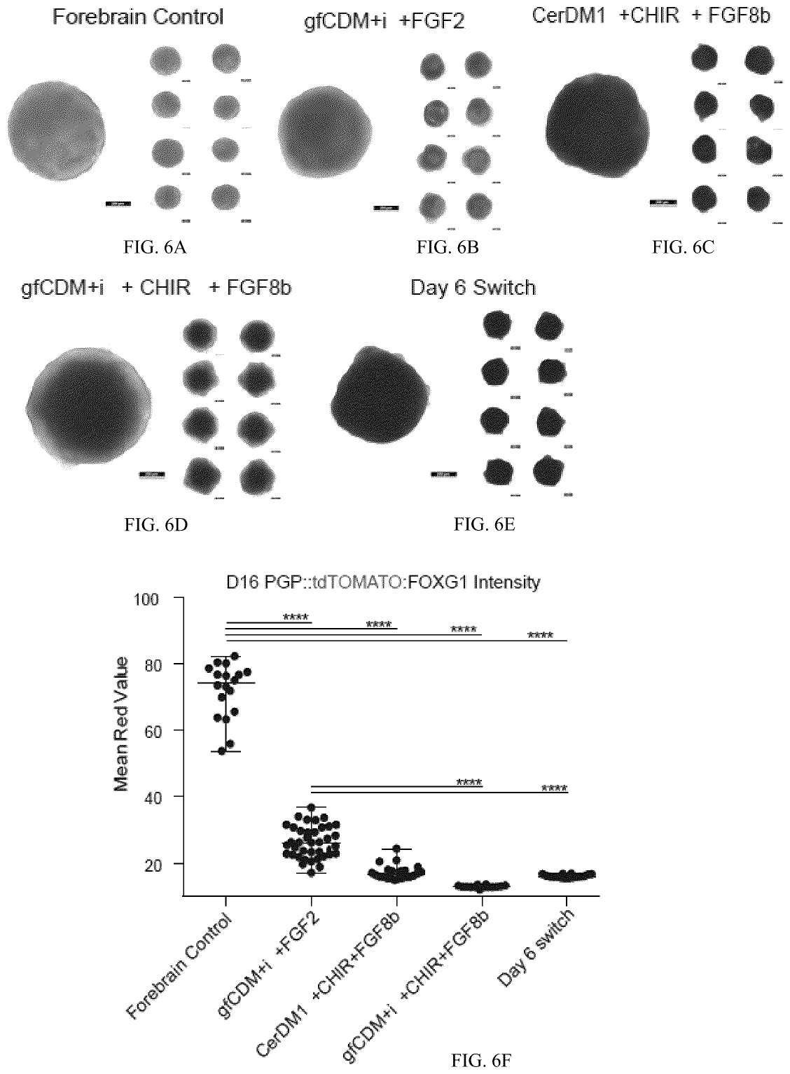

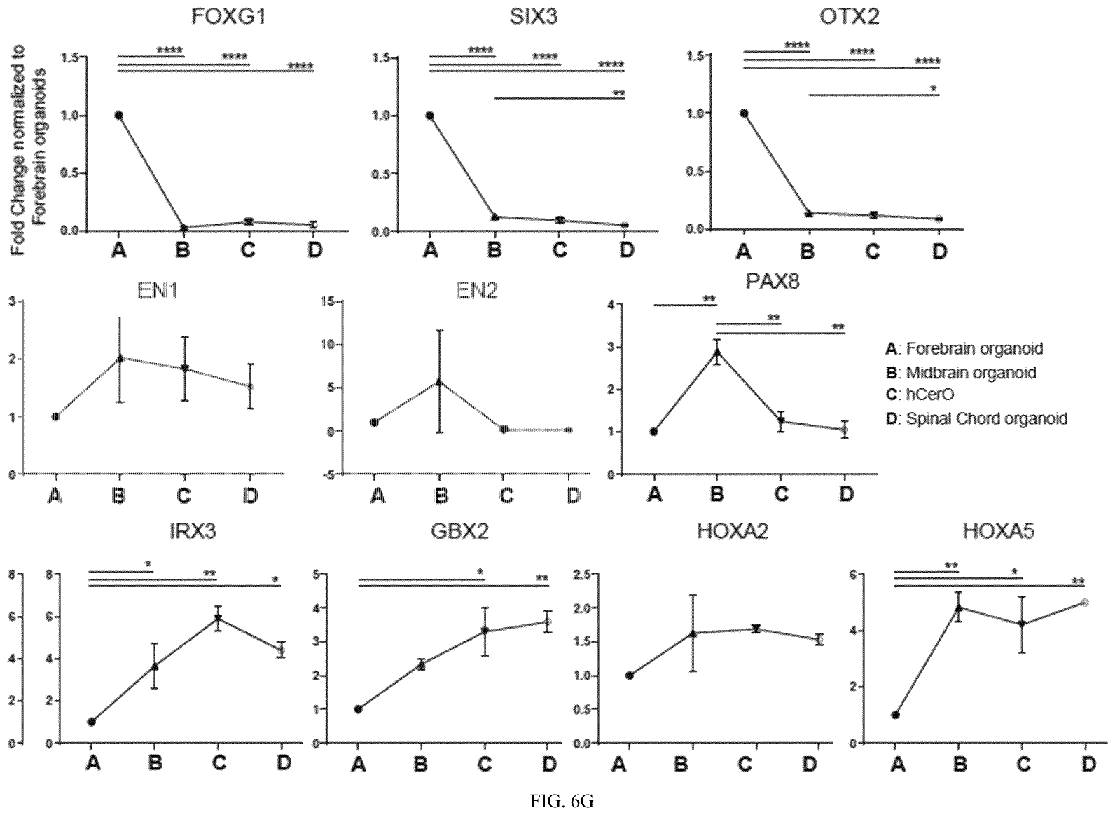

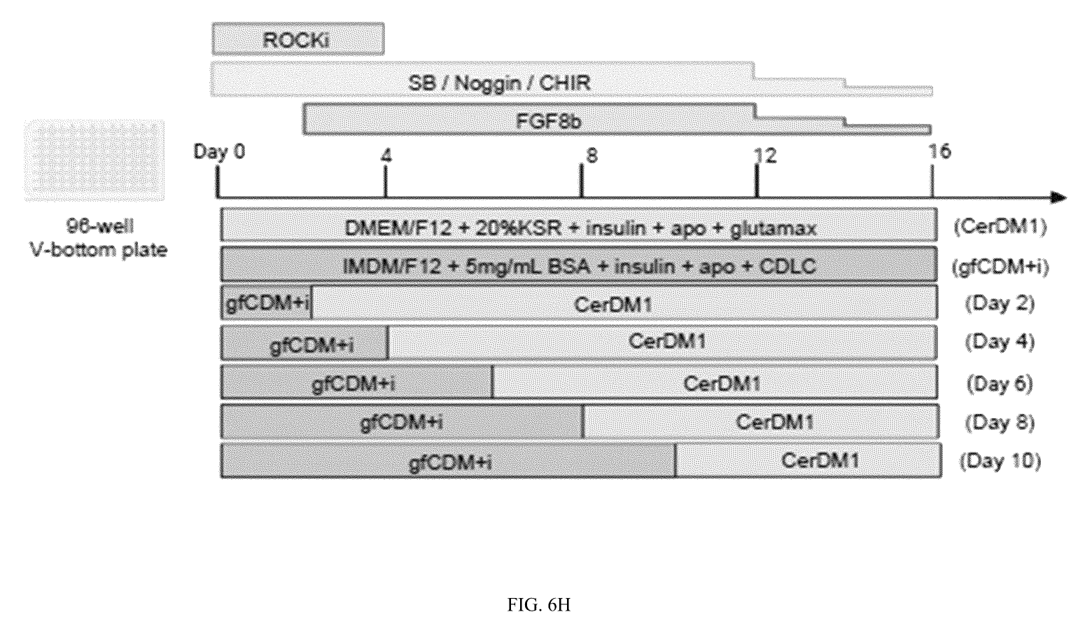

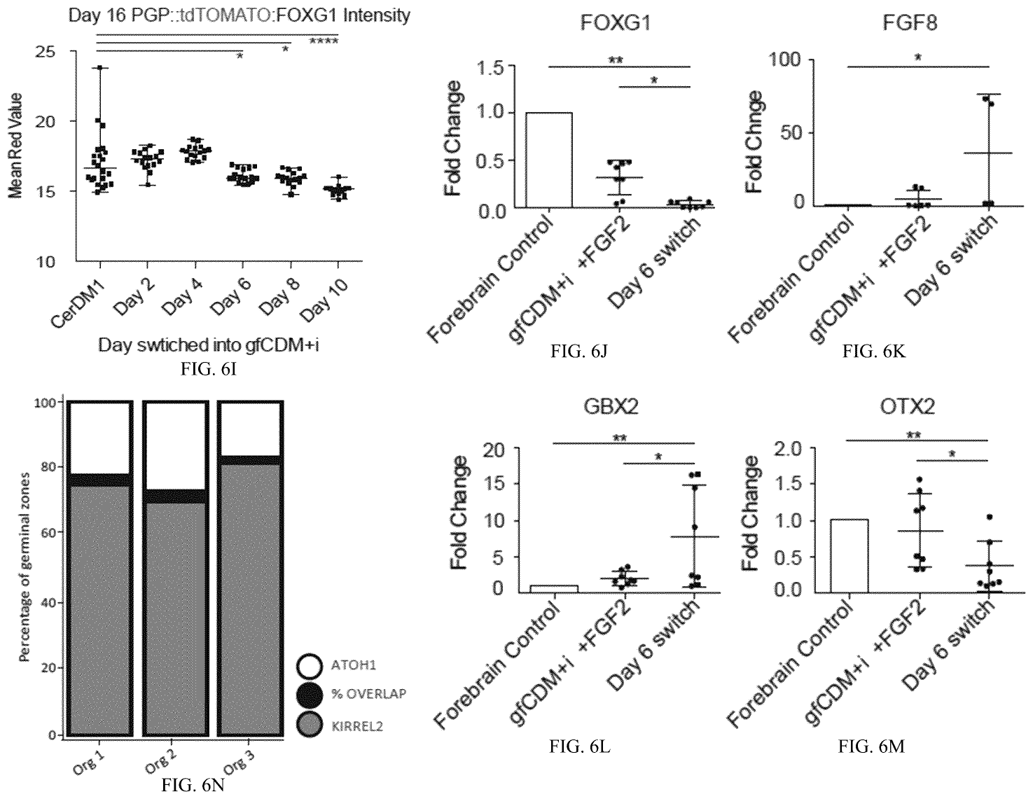

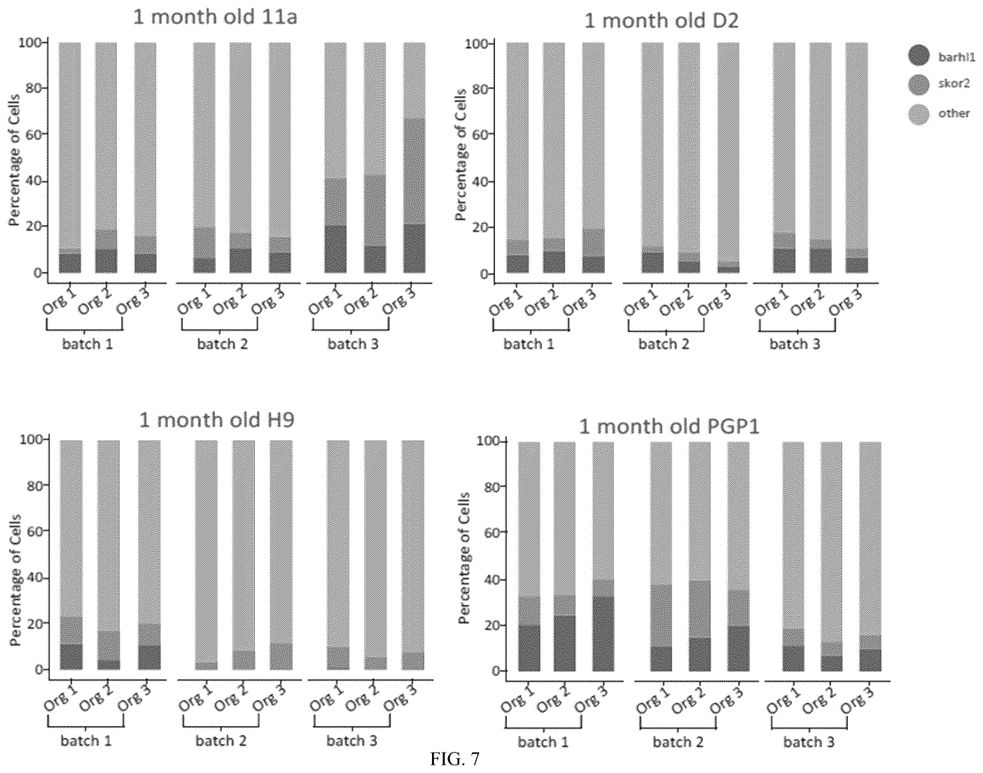

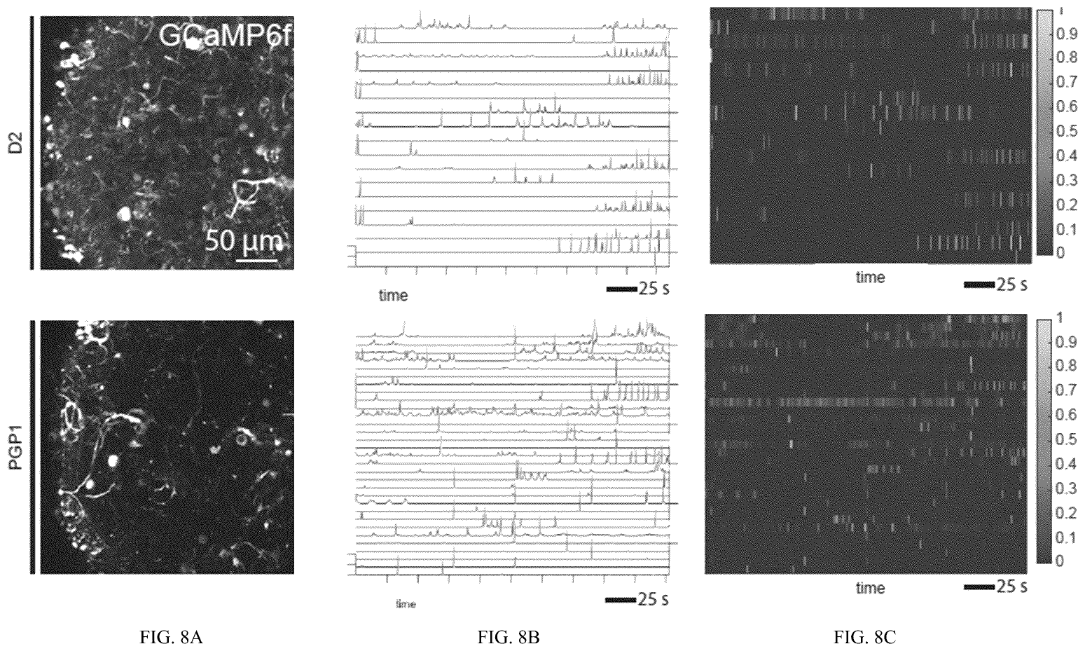

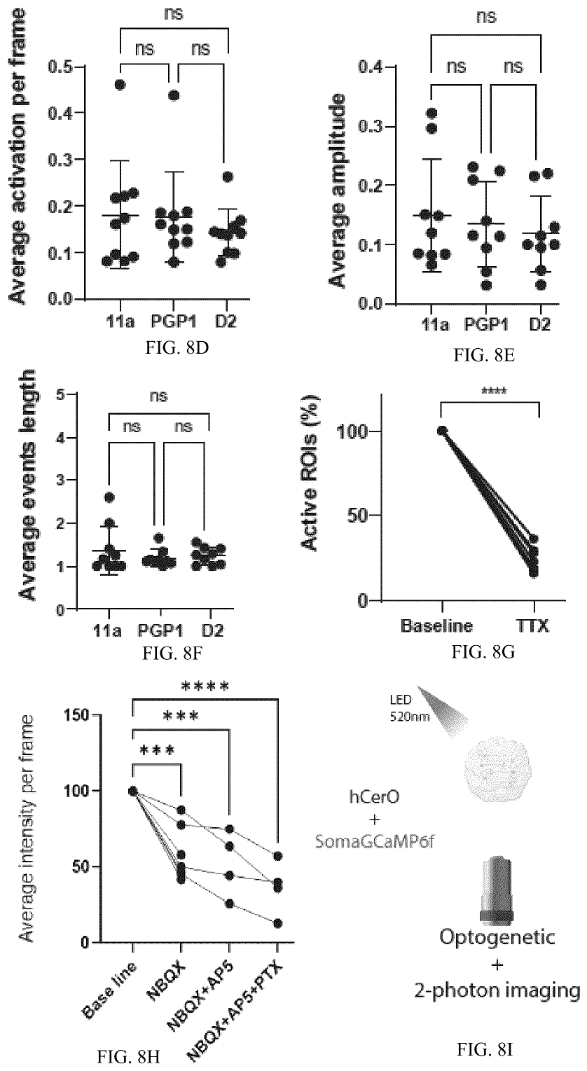

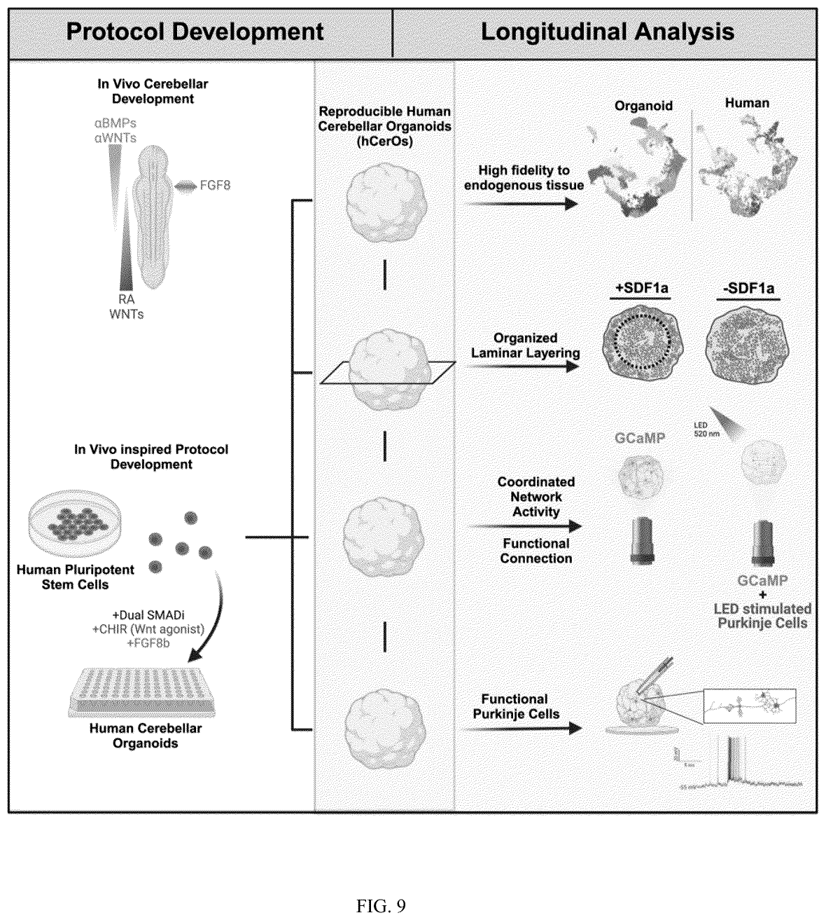

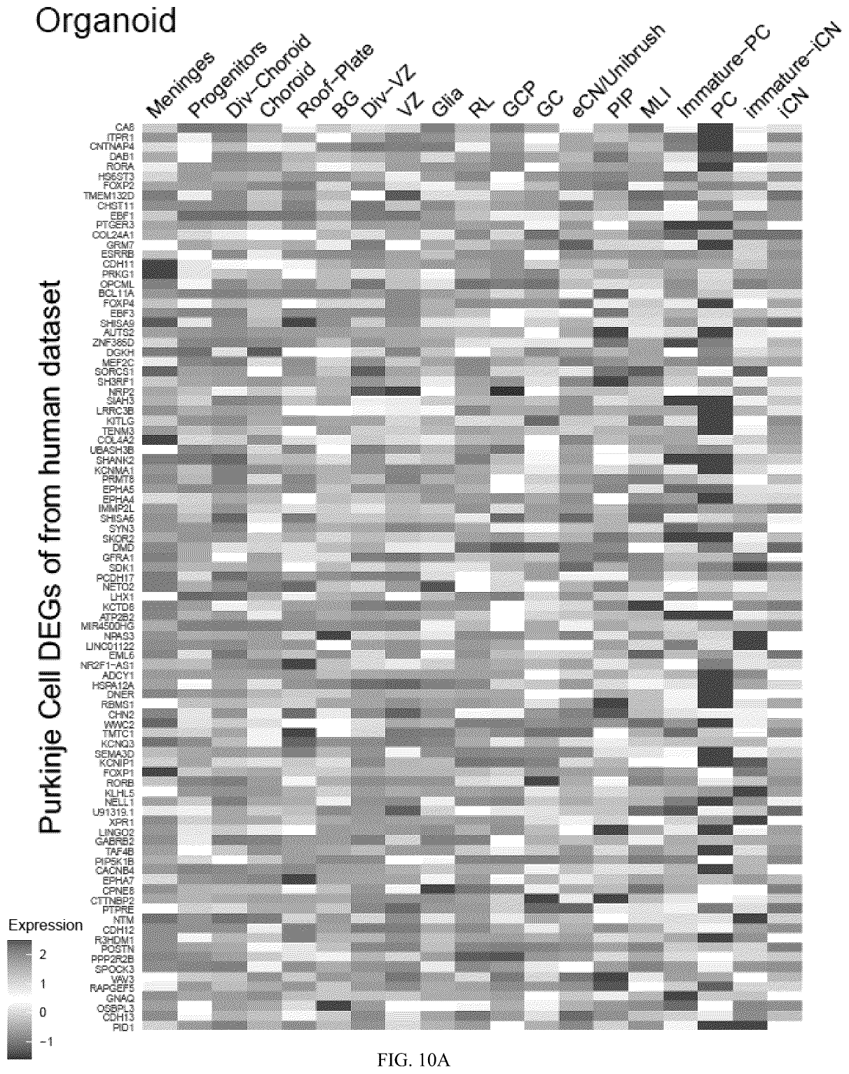

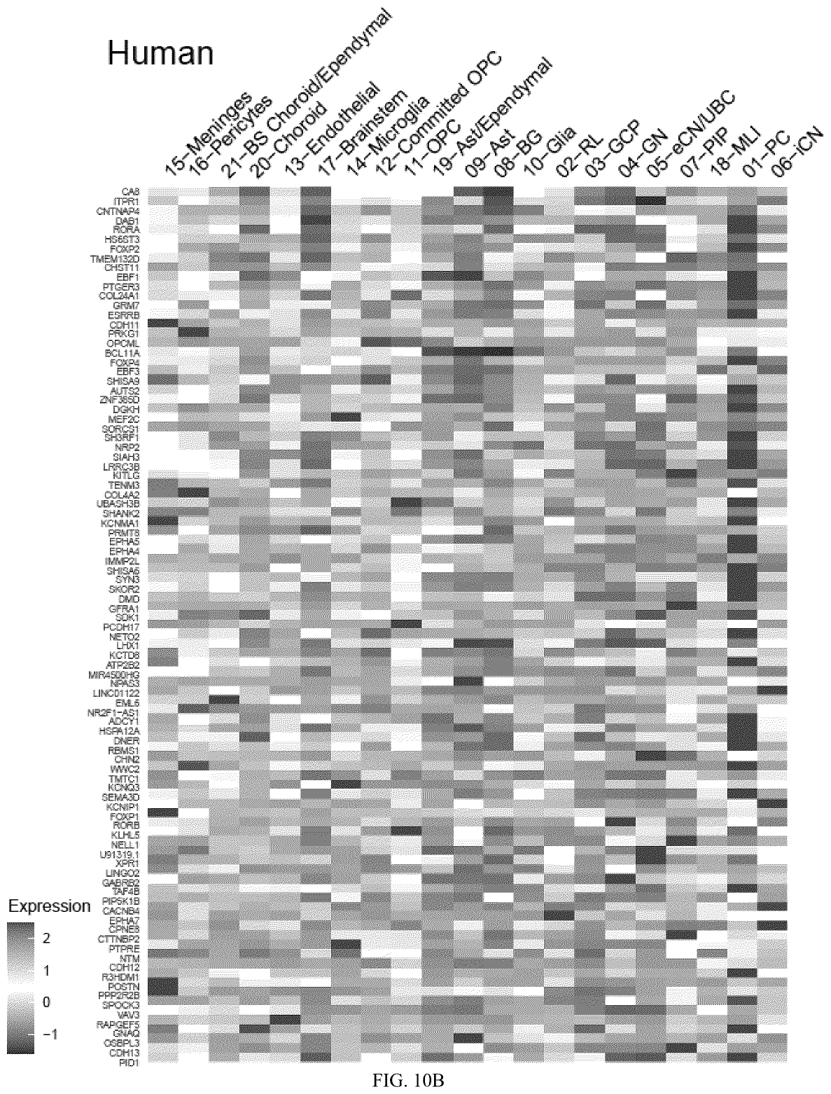

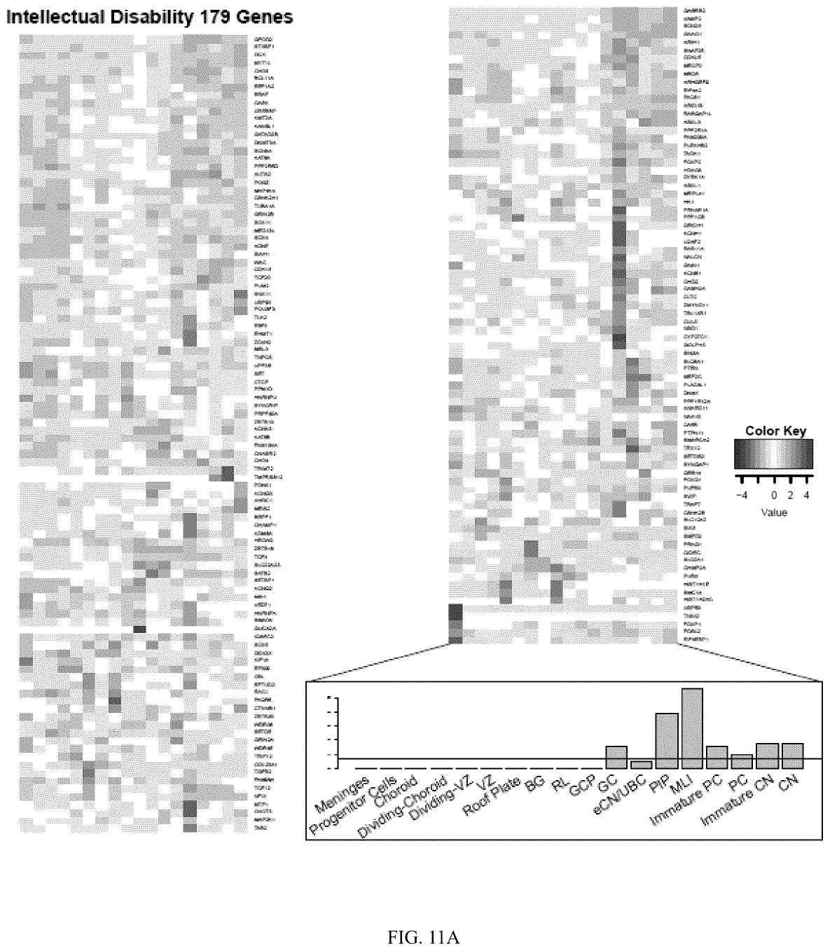

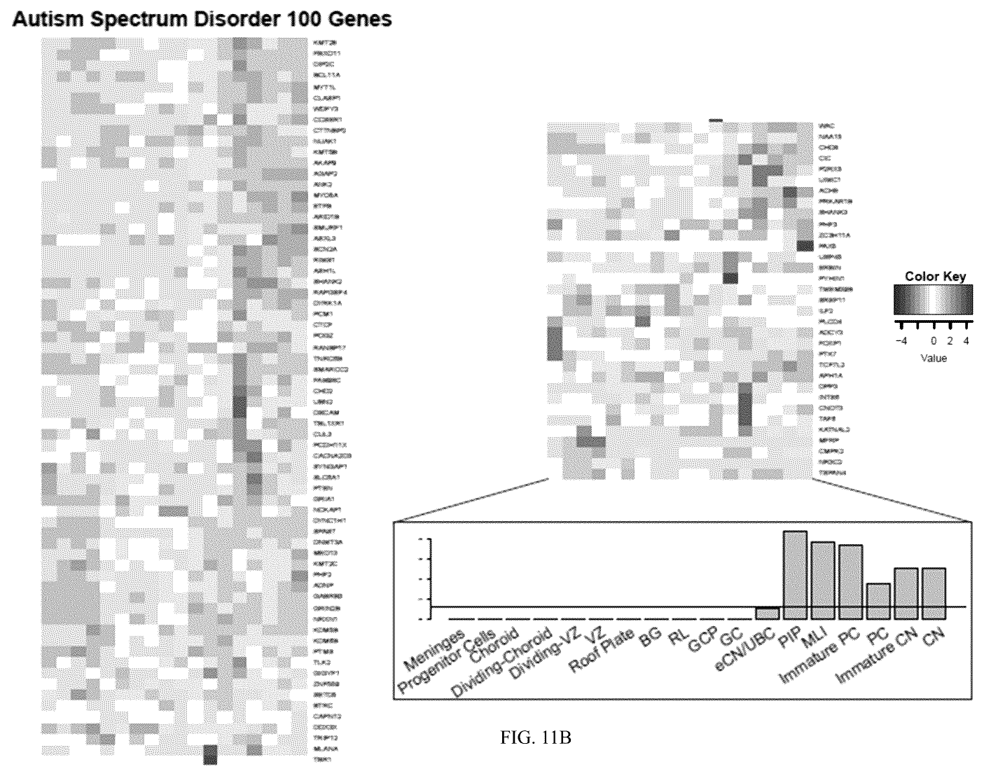

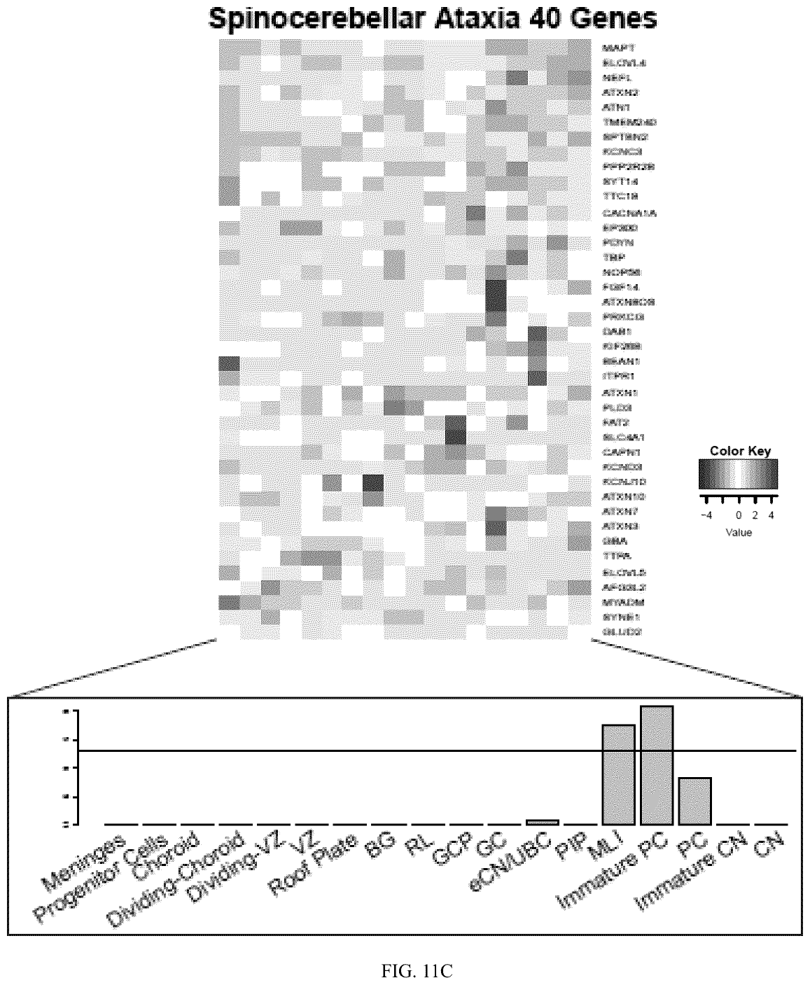

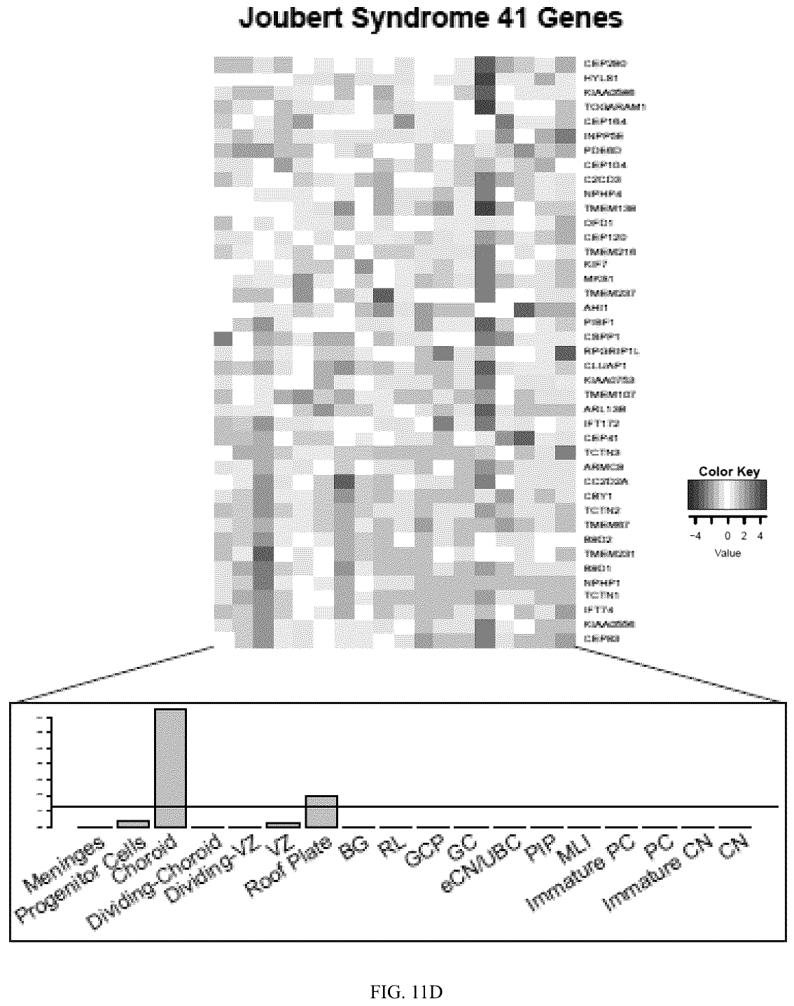

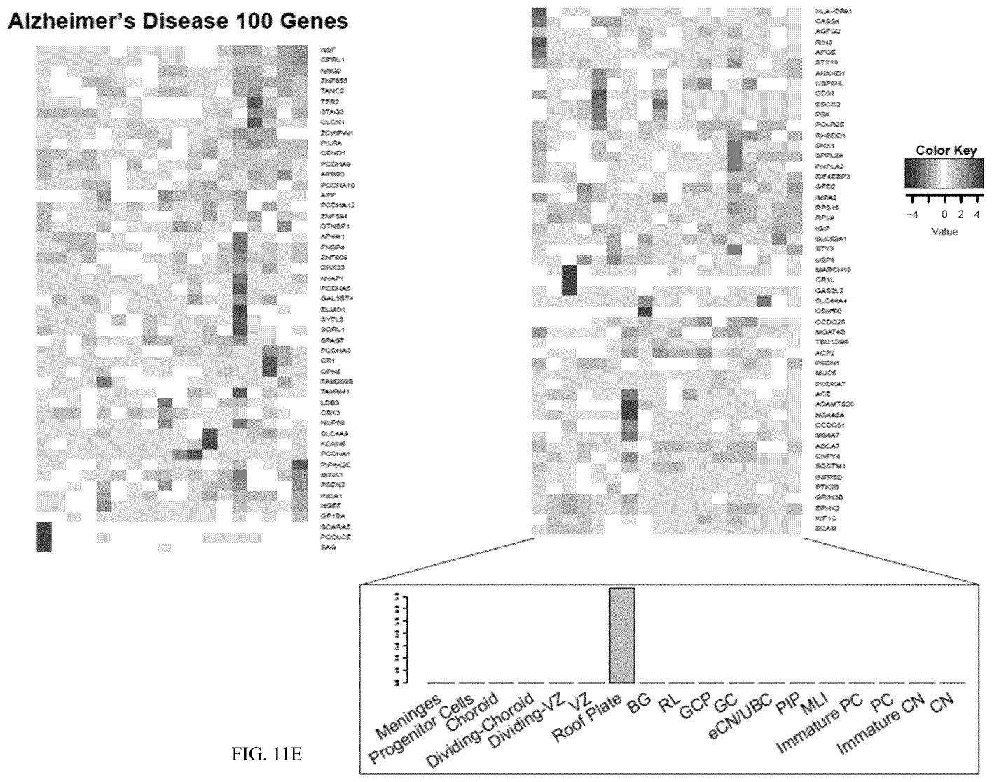

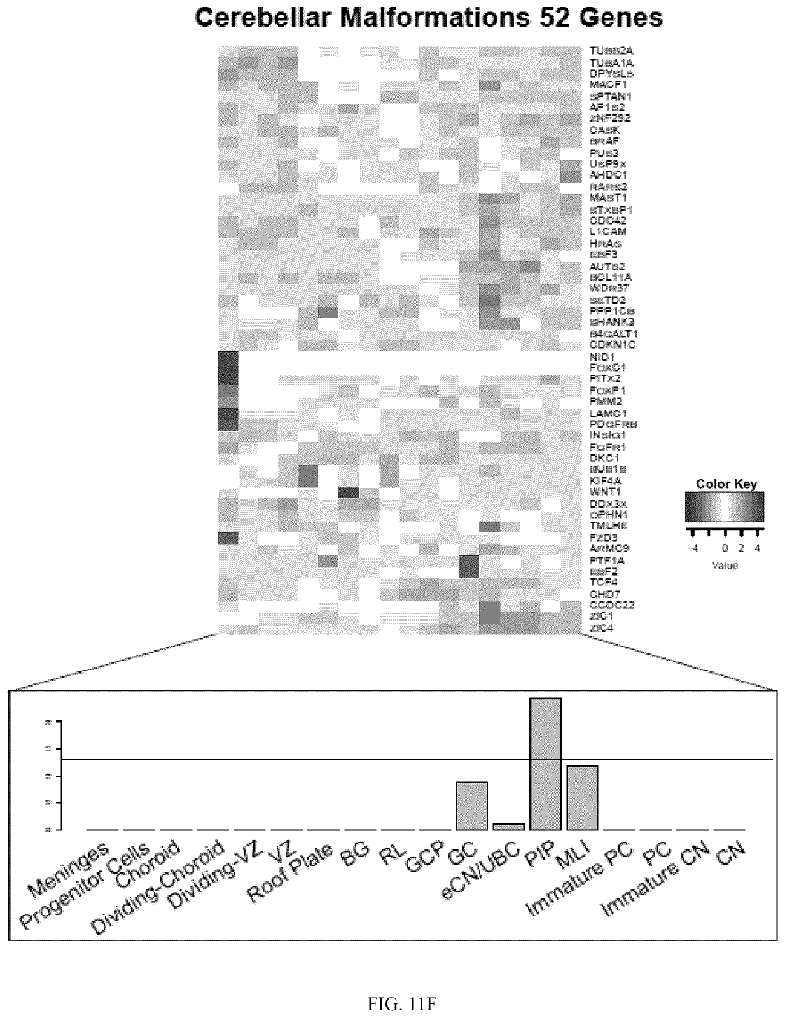

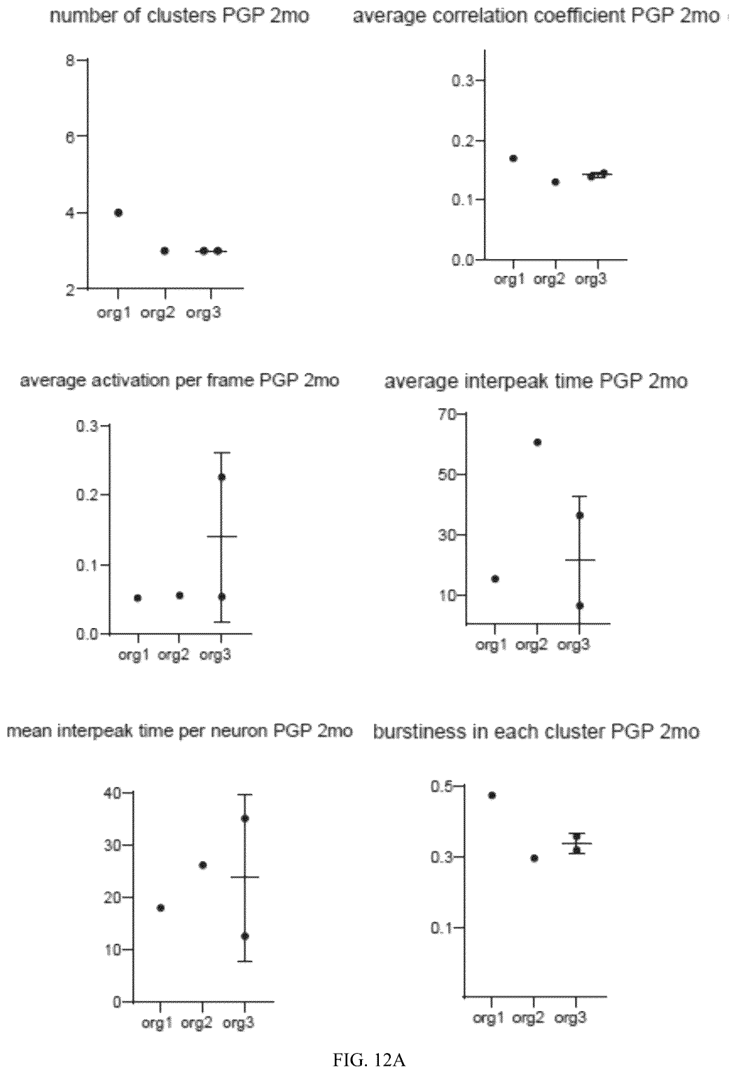

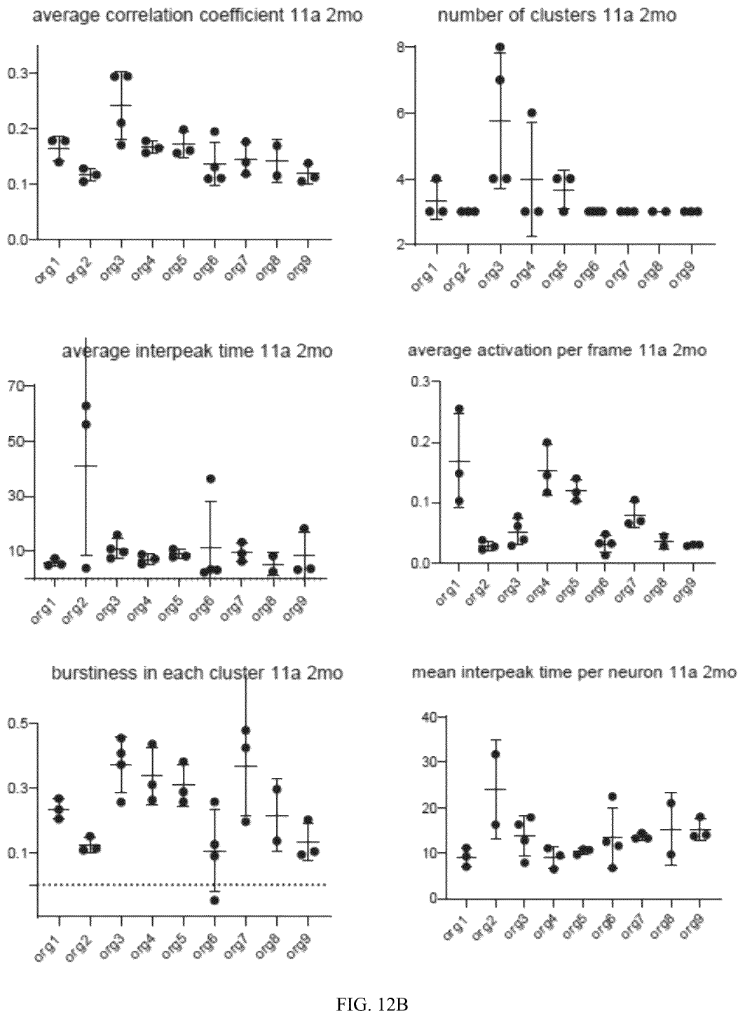

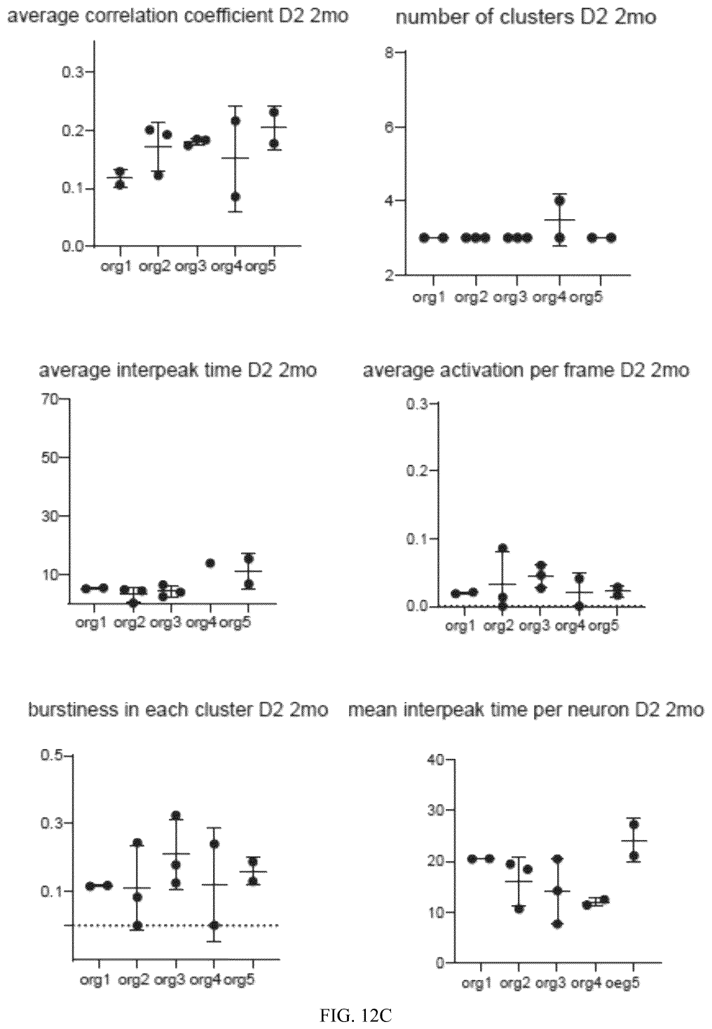

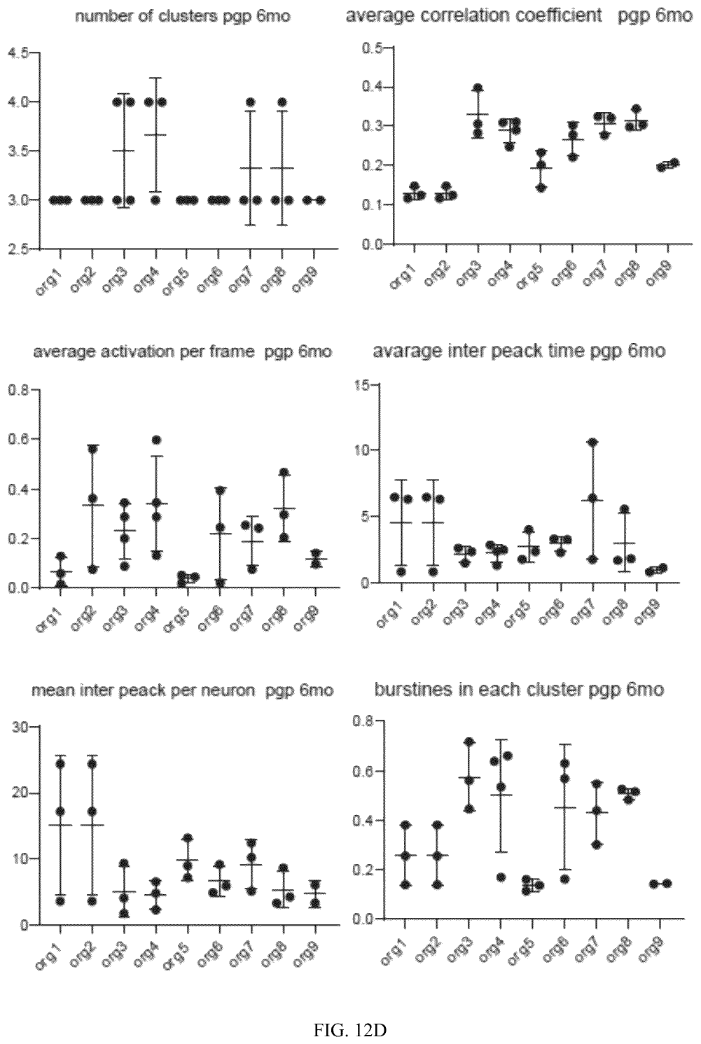

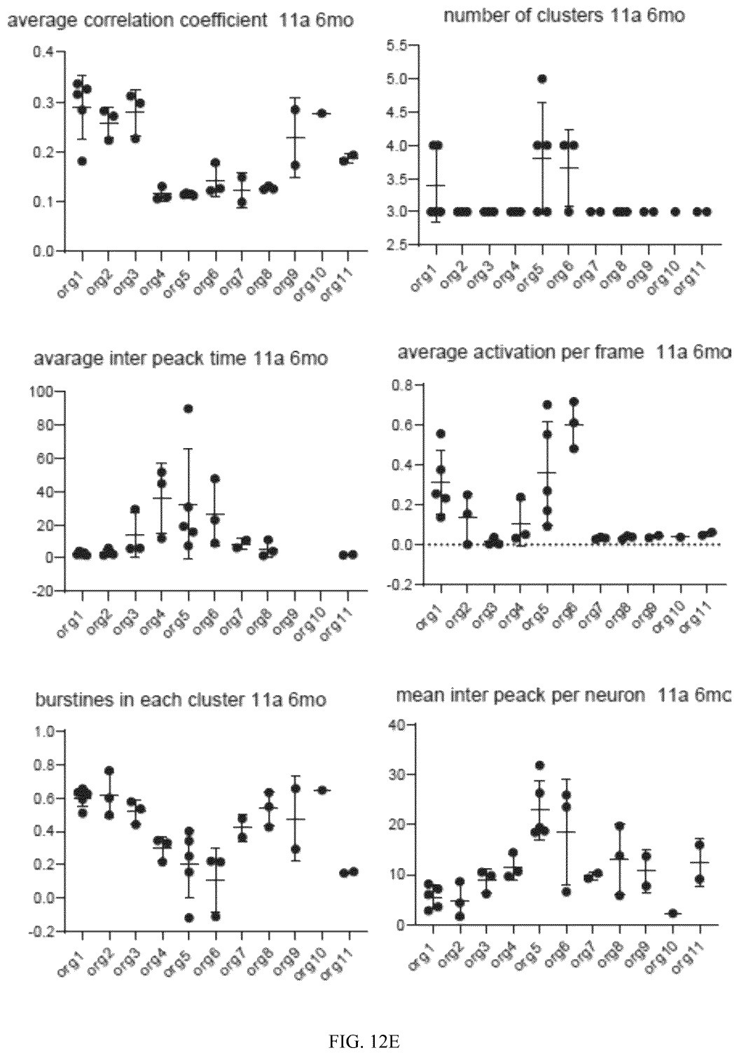

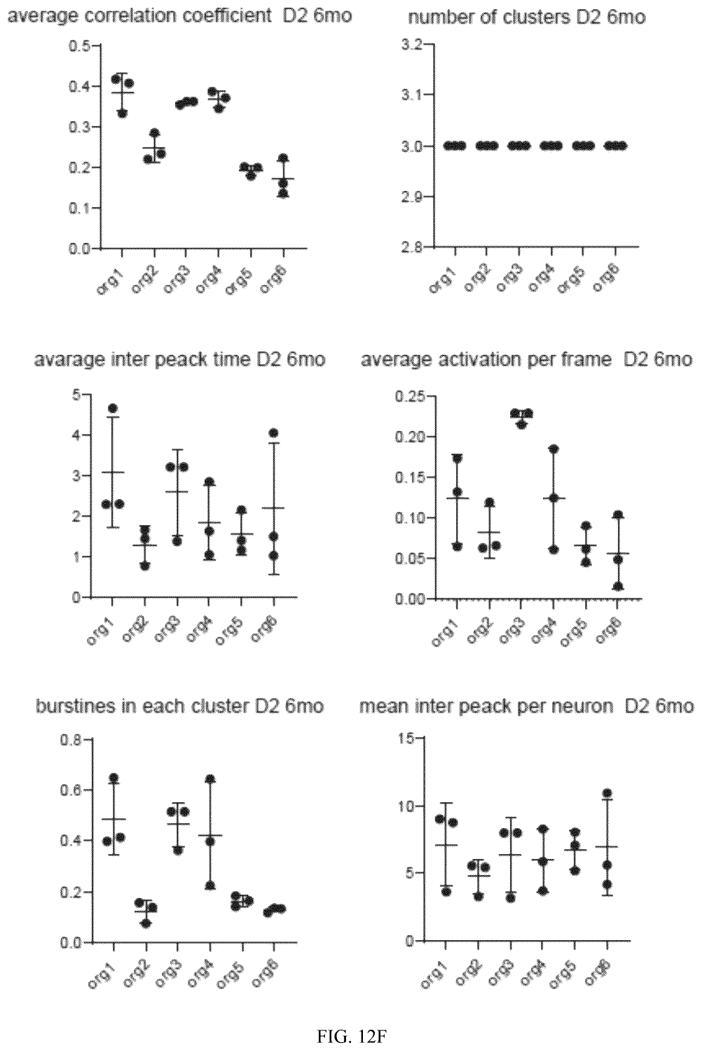

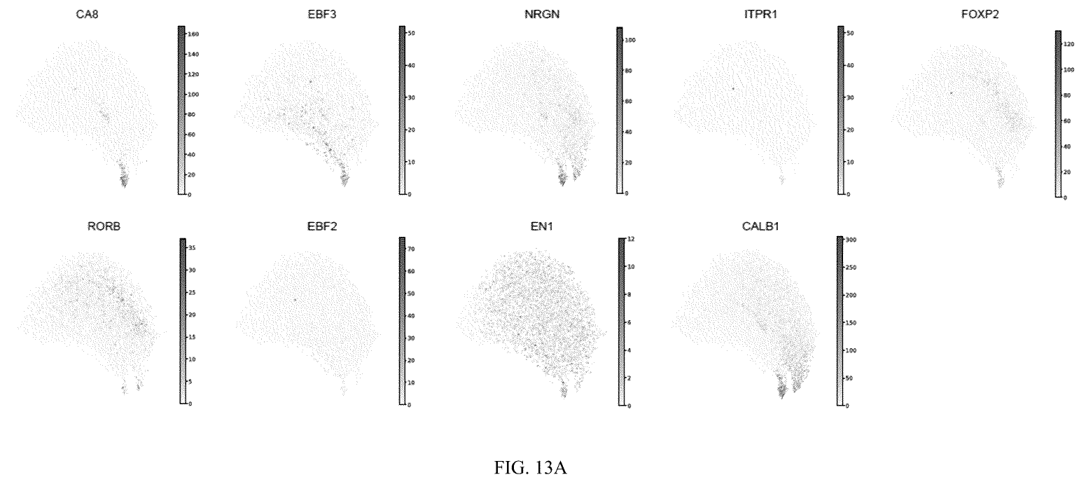

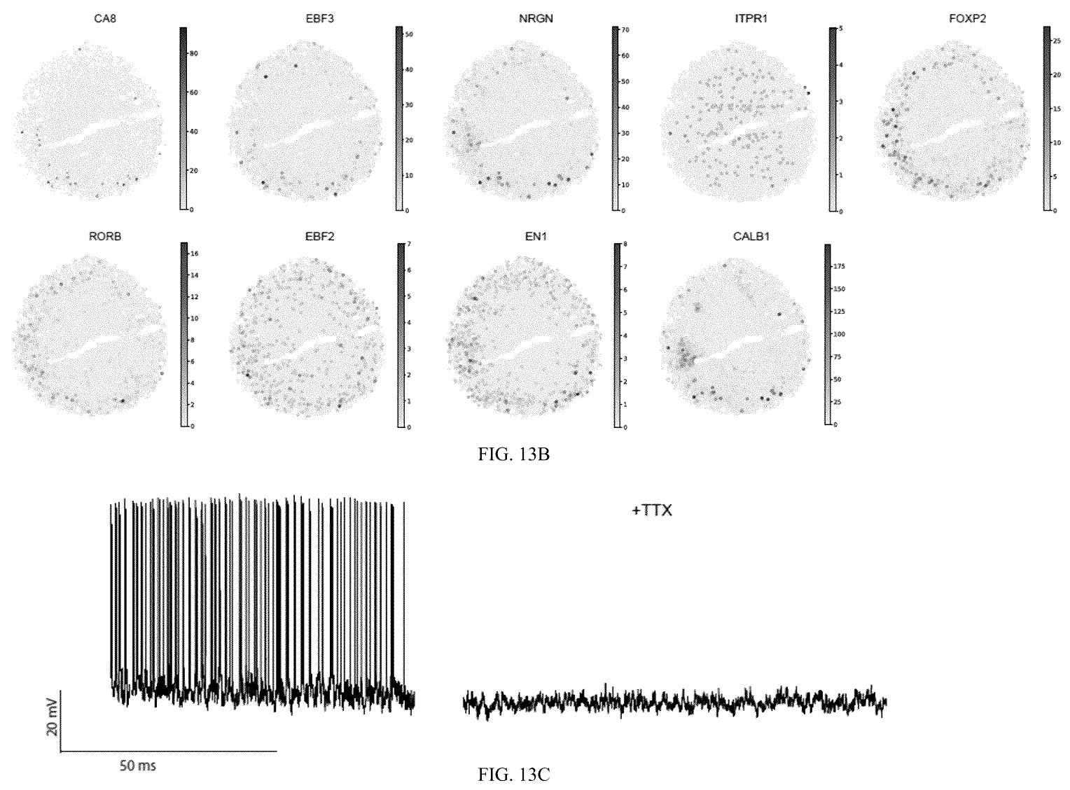

The original protocol, which involves switching to CerDM1 at day 6 of differentiation generates both progenitor cell types of the developing cerebellum (ATOH1 and KIRREL2) as well as its respective neuronal progenitors (BARHL1 and SKOR2). Maintaining the organoids in gfCDM for the duration of patterning (day 0-16) biases the cells to only generate the more dorsal granule cell progenitors of the developing cerebellar anlage that are ATOH1+ and BARHL1+(FIG. 3D). Additionally, these organoids maintain the lack of expression of FOXG1, a key forebrain marker as is indicated by the reporter cell line, therefore generating a purely caudal population of cells. As these organoids grow to day 60, it is possible to start noticing that these large, smooth, and round balls also have a textured portion to it, which is often an indicator of an emerging choroid plexus-like structure indicated by black arrows. This, however, may vary from cell line to cell line as the development of this region in culture is not specifically targeted. It is important to note that these structures do not make up more than 25% of the organoid as this may change the overall composition of the hCerOs and may vary between cell lines as the patterning strategy does not explicitly specify for the generation of this region in culture.

After 1 month in culture, immunohistochemical analysis, dissociation of organoids to single cells for single-cell or single-nuclei RNA sequencing, whole organoid GCaMP analysis and Patch-Clamp recordings will elucidate the composition and characteristics of these hCerOs.

At day 60 in culture, the Purkinje Cells start to express more mature Purkinje Cell markers such as CALB1 and have a simple dendritic morphology of a single linear dendritic outgrowth. However, it is also important to make sure that common markers used to identify excitatory cerebellar progenitors such as PAX6 and TBR1 at day 60 are not instead identifying forebrain cells (identified by the expression of FOXG1). One clear indication of the presence of forebrain within the cerebellar organoids are regions that are PAX6+TBR1+BARHL1−. In order for cells to be cerebellar, they must have PAX6 and BARHL1 in close proximity or colocalizing within a region of the organoid. As the Purkinje Cells continue on in culture, they start to increase in their dendritic arborization and complexity as they approach 6 months in culture. The maturation of Purkinje Cells in hCerOs can be further confirmed by various functional assays such as the study of calcium dynamics, whole-cell Patch Clamp recordings of intact organoids, and spatial transcriptomics to elucidate the interconnected and dynamic network that forms within these organoids.

TABLE 4
Major checkpoints.

| Checkpoint day | Question | Continue | Terminate | IHC markers |
|---|---|---|---|---|
| Day 1 or 2 | Have the hPSCs | Yes | No | N/A |
| | aggregated into a tight | | Make sure you terminate | |
| | EB with only a small | | only if there is a | |
| | amount of cell death | | significant number of | |
| | surrounding the central | | cells surrounding the | |
| | EB? | | central ball. (See FigX) | |
| Day 17 | Are the organoids intact | Yes | No | GBX2, OTX2 |
| | after Day 16 transfer? | Make sure they also | (Unhealthy organoids | |
| | | express more caudal | often start to disintegrate | |
| | | markers (GBX2) | and no longer have | |
| | | than rostral markers | distinct borders.) | |
| | | (OTX2) | | |
| Day 30 | Is the aggregate | Yes | No | SOX2, ATOH1, |
| | forming a clear | | (Make sure there aren't | KIRREL2 |
| | neuroepithelial layer | | smaller rosette structures | |
| | with both progenitor | | within the organoid | |
| | populations? | | prior to terminating by | |
| | | | IHC of cryosectioned | |
| | | | organoids.) | |
| | Do the organoid | Yes | No | BARHL1, |
| | express both excitatory | | (Not having one cell | SKOR2 |
| | and inhibitory neuronal | | type may not | |
| | markers? | | compromise the health | |
| | | | of the organoid, but may | |
| | | | affect the maturation | |
| | | | rate.) | |
| Day 60 | Do the organoids | Yes | No | CALB1, PAX6 |
| | express more mature | | | |
| | neuronal markers? | | | |

TABLE 5
Differentially expressed, selected genes in each of the
identified clusters of the cerebellum-like organoid.

| | p_val | avg_diff | pct. 1 | pct. 2 | p_val_adj | cluster | gene |
|---|---|---|---|---|---|---|---|
| LHX1 | 1.26E−121 | 13.235382 | 0.949 | 0.102 | 3.79E−118 | Immature-PC | LHX1 |
| CELF4 | 6.83E−110 | 7.6801961 | 0.888 | 0.187 | 2.05E−106 | Immature-PC | CELF4 |
| NEFL | 3.74E−101 | 9.6395589 | 0.79 | 0.135 | 1.12E−97 | Immature-PC | NEFL |
| POU3F1 | 1.21E−100 | 7.9510235 | 0.659 | 0.037 | 3.63E−97 | Immature-PC | POU3F1 |
| NEFM | 1.71E−100 | 9.2662563 | 0.813 | 0.175 | 5.14E−97 | Immature-PC | NEFM |
| AMPH | 4.28E−92 | 3.8067588 | 0.575 | 0.115 | 1.29E−88 | Immature-PC | AMPH |
| ZNF385D | 1.14E−91 | 5.0222552 | 0.724 | 0.144 | 3.42E−88 | Immature-PC | ZNF385D |
| PBX3 | 1.26E−91 | 4.3975711 | 0.836 | 0.276 | 3.78E−88 | Immature-PC | PBX3 |
| SKOR2 | 5.48E−67 | 2.1887905 | 0.252 | 0.033 | 1.64E−63 | Immature-PC | SKOR2 |
| GAD1 | 3.50E−65 | 2.1083621 | 0.374 | 0.094 | 1.05E−61 | Immature-PC | GAD1 |
| ZFHX31 | 9.17E−12 | 7.5549199 | 1 | 0.232 | 2.75E−08 | MLI | ZFHX3 |
| LUZP2 | 2.77E−11 | 10.584371 | 0.941 | 0.065 | 8.31E−08 | MLI | LUZP2 |
| ZFHX41 | 3.02E−11 | 6.5274419 | 1 | 0.28 | 9.05E−08 | MLI | ZFHX4 |
| PKIA1 | 3.55E−11 | 6.1884784 | 1 | 0.305 | 1.06E−07 | MLI | PKIA |
| LHX11 | 1.69E−10 | 8.0559417 | 1 | 0.113 | 5.06E−07 | MLI | LHX1 |
| SOX41 | 2.78E−10 | 12.056893 | 0.941 | 0.305 | 8.35E−07 | MLI | SOX4 |
| THSD7A1 | 3.59E−10 | 6.4502625 | 0.941 | 0.228 | 1.08E−06 | MLI | THSD7A |
| STMN41 | 4.22E−08 | 4.3798813 | 0.941 | 0.297 | 0.0001266 | MLI | STMN4 |
| DMBX1 | 4.40E−08 | 9.0641764 | 0.471 | 0.005 | 0.0001321 | MLI | DMBX1 |
| SOX14 | 1.95E−07 | 9.3541164 | 0.471 | 0.004 | 0.0005842 | MLI | SOX14 |
| NUSAP1 | 1.13E−109 | 5.7688472 | 0.933 | 0.27 | 3.39E−106 | Div-VZ | NUSAP1 |
| TYMS | 1.85E−108 | 4.9101668 | 0.909 | 0.216 | 5.55E−105 | Div-VZ | TYMS |
| DEK | 1.12E−106 | 2.9409887 | 0.957 | 0.364 | 3.36E−103 | Div-VZ | DEK |
| HIST1H3B | 2.64E−104 | 8.1367976 | 0.874 | 0.085 | 7.93E−101 | Div-VZ | HIST1H3B |
| CLSPN | 8.04E−104 | 2.7175689 | 0.798 | 0.15 | 2.41E−100 | Div-VZ | CLSPN |
| TOP2A | 2.13E−84 | 4.7759138 | 0.881 | 0.295 | 6.39E−81 | Div-VZ | TOP2A |
| HES4 | 1.31E−81 | 4.1739192 | 0.862 | 0.285 | 3.93E−78 | Div-VZ | HES4 |
| ATAD2 | 1.35E−77 | 2.9822694 | 0.711 | 0.112 | 4.04E−74 | Div-VZ | ATAD2 |
| MKI67 | 3.48E−72 | 4.1675962 | 0.85 | 0.278 | 1.04E−68 | Div-VZ | MKI67 |
| VIM | 2.42E−65 | 2.8043327 | 0.866 | 0.299 | 7.27E−62 | Div-VZ | VIM |
| NEUROD1 | 6.68E−54 | 8.1637717 | 0.787 | 0.081 | 2.01E−50 | eCN/Unibrush | NEUROD1 |
| SSTR2 | 6.86E−44 | 5.2145696 | 0.811 | 0.212 | 2.06E−40 | eCN/Unibrush | SSTR2 |
| GRIA22 | 9.60E−42 | 5.3685056 | 0.672 | 0.137 | 2.88E−38 | eCN/Unibrush | GRIA2 |
| PAX61 | 3.58E−41 | 3.5548944 | 0.836 | 0.292 | 1.08E−37 | eCN/Unibrush | PAX6 |
| STMN22 | 2.69E−39 | 8.3857363 | 0.861 | 0.3 | 8.06E−36 | eCN/Unibrush | STMN2 |
| CBLN1 | 7.75E−39 | 4.1040123 | 0.656 | 0.164 | 2.33E−35 | eCN/Unibrush | CBLN1 |
| SOX42 | 2.96E−38 | 4.1838159 | 0.893 | 0.301 | 8.87E−35 | eCN/Unibrush | SOX4 |
| BARHL1 | 4.10E−37 | 3.5729908 | 0.664 | 0.125 | 1.23E−33 | eCN/Unibrush | BARHL1 |
| TAGLN33 | 2.15E−36 | 2.6515289 | 0.803 | 0.347 | 6.46E−33 | eCN/Unibrush | TAGLN3 |
| EOMES | 9.53E−16 | 5.2654972 | 0.311 | 0.006 | 2.86E−12 | eCN/Unibrush | EOMES |
| DEK1 | 1.29E−177 | 3.5611235 | 0.975 | 0.36 | 3.86E−174 | RL | DEK |
| PTMA3 | 1.55E−173 | 3.6517228 | 0.98 | 0.378 | 4.65E−170 | RL | PTMA |
| IGFBPL11 | 6.78E−165 | 6.8050027 | 0.958 | 0.218 | 2.03E−161 | RL | IGFBPL1 |
| CENPV2 | 3.66E−162 | 3.5562078 | 0.953 | 0.362 | 1.10E−158 | RL | CENPV |
| TYMS1 | 2.23E−156 | 4.7779547 | 0.93 | 0.211 | 6.69E−153 | RL | TYMS |
| HES61 | 2.65E−155 | 9.5018343 | 0.916 | 0.161 | 7.95E−152 | RL | HES6 |
| CLSPN1 | 3.78E−152 | 4.2269945 | 0.855 | 0.144 | 1.13E−148 | RL | CLSPN |
| MKI671 | 2.64E−147 | 5.2410746 | 0.925 | 0.272 | 7.92E−144 | RL | MKI67 |
| TOP2A1 | 1.02E−139 | 5.1197071 | 0.916 | 0.289 | 3.05E−136 | RL | TOP2A |
| ATOH1 | 2.60E−18 | 1.6699642 | 0.45 | 0.058 | 7.80E−15 | RL | ATOH1 |
| COL1A1 | 4.31E−231 | 22.663986 | 1 | 0.028 | 1.29E−227 | Meninges | COL1A1 |
| COL3A1 | 5.11E−231 | 22.66363 | 1 | 0.025 | 1.53E−227 | Meninges | COL3A1 |
| COL1A2 | 3.20E−230 | 21.562654 | 0.997 | 0.137 | 9.60E−227 | Meninges | COL1A2 |
| LGALS1 | 9.58E−230 | 21.255325 | 0.997 | 0.144 | 2.87E−226 | Meninges | LGALS1 |
| DCN | 4.49E−229 | 20.536441 | 0.994 | 0.032 | 1.35E−225 | Meninges | DCN |
| COL6A3 | 6.70E−227 | 19.51619 | 0.981 | 0.013 | 2.01E−223 | Meninges | COL6A3 |
| APOE | 3.85E−222 | 19.539411 | 0.994 | 0.202 | 1.15E−218 | Meninges | APOE |
| TIMP1 | 2.94E−209 | 13.390883 | 0.964 | 0.139 | 8.81E−206 | Meninges | TIMP1 |
| COL4A21 | 6.76E−204 | 10.249968 | 0.944 | 0.099 | 2.03E−200 | Meninges | COL4A2 |
| LUM | 9.72E−201 | 13.43535 | 0.939 | 0.038 | 2.92E−197 | Meninges | LUM |
| TOP2A3 | 3.98E−281 | 3.9196002 | 0.951 | 0.272 | 1.20E−277 | Div-Choroid | TOP2A |
| HMGB22 | 6.14E−263 | 3.988237 | 0.939 | 0.245 | 1.84E−259 | Div-Choroid | HMGB2 |
| H2AFZ2 | 3.43E−253 | 2.6579475 | 0.932 | 0.313 | 1.03E−249 | Div-Choroid | H2AFZ |
| TYMS3 | 9.71E−247 | 3.5352092 | 0.869 | 0.196 | 2.91E−243 | Div-Choroid | TYMS |
| SMC42 | 2.15E−237 | 2.2269521 | 0.901 | 0.235 | 6.46E−234 | Div-Choroid | SMC4 |
| TUBA1B3 | 5.03E−221 | 2.8102722 | 0.901 | 0.289 | 1.51E−217 | Div-Choroid | TUBA1B |
| ZWINT3 | 1.78E−216 | 2.1251535 | 0.708 | 0.113 | 5.33E−213 | Div-Choroid | ZWINT |
| MKI672 | 6.02E−211 | 2.5871197 | 0.885 | 0.258 | 1.80E−207 | Div-Choroid | MKI67 |
| TTR | 2.01E−41 | 3.6640719 | 0.399 | 0.174 | 6.03E−38 | Div-Choroid | TTR |
| MDK3 | 3.17E−40 | 0.5793004 | 0.57 | 0.377 | 9.51E−37 | Div-Choroid | MDK |
| PTN2 | 3.50E−235 | 19.160351 | 0.998 | 0.228 | 1.05E−231 | VZ | PTN |
| SLC1A32 | 4.76E−234 | 8.3535451 | 0.979 | 0.182 | 1.43E−230 | VZ | SLC1A3 |
| TTYH11 | 1.55E−233 | 11.52003 | 0.995 | 0.195 | 4.64E−230 | VZ | TTYH1 |
| VIM2 | 6.79E−221 | 9.4275372 | 0.991 | 0.288 | 2.04E−217 | VZ | VIM |
| HES5 | 9.30E−188 | 9.0456809 | 0.898 | 0.126 | 2.79E−184 | VZ | HES5 |
| PTPRZ11 | 3.04E−179 | 4.8072279 | 0.935 | 0.223 | 9.12E−176 | VZ | PTPRZ1 |
| ID42 | 5.66E−175 | 4.4546271 | 0.928 | 0.274 | 1.70E−171 | VZ | ID4 |
| NES1 | 8.92E−175 | 4.3827825 | 0.933 | 0.232 | 2.68E−171 | VZ | NES |
| FABP71 | 1.07E−158 | 6.1794927 | 0.868 | 0.202 | 3.22E−155 | VZ | FABP7 |
| EDNRB | 1.23E−154 | 5.7447502 | 0.833 | 0.121 | 3.69E−151 | VZ | EDNRB |
| SPARCL1 | 0 | 14.77377 | 0.911 | 0.167 | 0 | Roof-Plate | SPARCL1 |
| RSPO3 | 0 | 8.3496668 | 0.868 | 0.158 | 0 | Roof-Plate | RSPO3 |
| WLS1 | 0 | 4.5363817 | 0.926 | 0.292 | 0 | Roof-Plate | WLS |
| ZFHX42 | 2.58E−307 | 4.8087086 | 0.856 | 0.246 | 7.74E−304 | Roof-Plate | ZFHX4 |
| RMST1 | 1.28E−293 | 4.0003881 | 0.86 | 0.271 | 3.83E−290 | Roof-Plate | RMST |
| PON21 | 1.62E−267 | 3.2243529 | 0.844 | 0.259 | 4.85E−264 | Roof-Plate | PON2 |
| LY6H1 | 3.39E−236 | 2.7242193 | 0.841 | 0.354 | 1.02E−232 | Roof-Plate | LY6H |
| NES2 | 3.51E−207 | 4.2106846 | 0.758 | 0.222 | 1.05E−203 | Roof-Plate | NES |
| EFNB32 | 7.93E−204 | 3.9568824 | 0.77 | 0.239 | 2.38E−200 | Roof-Plate | EFNB3 |
| GDF71 | 3.56E−63 | 1.6735906 | 0.504 | 0.11 | 1.07E−59 | Roof-Plate | GDF7 |
| MT-CO12 | 1.03E−306 | 1.200755 | 0.797 | 0.409 | 3.08E−303 | Choroid | MT-CO1 |
| CLDN51 | 2.27E−300 | 2.5079099 | 0.657 | 0.209 | 6.82E−297 | Choroid | CLDN5 |
| ZFP36L21 | 5.18E−297 | 1.2902833 | 0.654 | 0.252 | 1.55E−293 | Choroid | ZFP36L2 |
| MT-CO31 | 8.22E−297 | 1.1994744 | 0.769 | 0.423 | 2.47E−293 | Choroid | MT-CO3 |
| SLC5A31 | 8.45E−286 | 1.3957054 | 0.679 | 0.262 | 2.54E−282 | Choroid | SLC5A3 |
| RBP12 | 9.16E−257 | 2.8005379 | 0.603 | 0.235 | 2.75E−253 | Choroid | RBP1 |
| NPC23 | 1.31E−256 | 0.9496959 | 0.702 | 0.33 | 3.92E−253 | Choroid | NPC2 |
| MT-ND11 | 2.14E−256 | 1.9444008 | 0.689 | 0.355 | 6.41E−253 | Choroid | MT-ND1 |
| KIF91 | 7.56E−256 | 1.8474548 | 0.644 | 0.235 | 2.27E−252 | Choroid | KIF9 |
| CLU3 | 7.72E−254 | 1.0857079 | 0.652 | 0.308 | 2.32E−250 | Choroid | CLU |
| TFAP2A2 | 0 | 6.1180681 | 0.749 | 0.093 | 0 | PIP | TFAP2A |
| GAD21 | 1.94E−307 | 9.8395521 | 0.743 | 0.061 | 5.81E−304 | PIP | GAD2 |
| TFAP2B2 | 3.58E−307 | 8.2189731 | 0.678 | 0.04 | 1.07E−303 | PIP | TFAP2B |
| SYT15 | 2.05E−244 | 6.356651 | 0.798 | 0.234 | 6.15E−241 | PIP | SYT1 |
| NRXN14 | 1.43E−236 | 5.3064482 | 0.834 | 0.269 | 4.30E−233 | PIP | NRXN1 |
| ELAVL43 | 1.65E−225 | 2.710845 | 0.571 | 0.143 | 4.95E−222 | PIP | ELAVL4 |
| NSG23 | 1.73E−213 | 2.96897 | 0.654 | 0.16 | 5.18E−210 | PIP | NSG2 |
| THSD7A3 | 3.44E−207 | 3.0587941 | 0.624 | 0.209 | 1.03E−203 | PIP | THSD7A |
| EBF2 | 3.05E−201 | 3.0538063 | 0.294 | 0.021 | 9.15E−198 | PIP | EBF2 |
| PAX2 | 3.16E−176 | 3.866404 | 0.243 | 0.007 | 9.49E−173 | PIP | PAX2 |
| FABP73 | 7.21E−266 | 12.306735 | 0.898 | 0.194 | 2.16E−262 | BG | FABP7 |
| TTYH13 | 2.18E−265 | 9.3627357 | 0.947 | 0.189 | 6.54E−262 | BG | TTYH1 |
| EDNRB2 | 3.17E−261 | 11.000904 | 0.803 | 0.115 | 9.52E−258 | BG | EDNRB |
| PTPRZ13 | 5.86E−246 | 7.9627035 | 0.829 | 0.22 | 1.76E−242 | BG | PTPRZ1 |
| C1orf612 | 5.06E−239 | 11.258141 | 0.853 | 0.209 | 1.52E−235 | BG | C1orf61 |
| VIM4 | 3.76E−232 | 7.8171726 | 0.916 | 0.284 | 1.13E−228 | BG | VIM |
| GPM6B3 | 2.48E−231 | 5.7847467 | 0.905 | 0.233 | 7.44E−228 | BG | GPM6B |
| PTN4 | 1.50E−230 | 13.396196 | 0.943 | 0.223 | 4.51E−227 | BG | PTN |
| WNT7B1 | 1.37E−210 | 6.8594199 | 0.621 | 0.041 | 4.10E−207 | BG | WNT7B |
| ID44 | 8.52E−201 | 5.0042148 | 0.853 | 0.27 | 2.55E−197 | BG | ID4 |
| TAC13 | 4.15E−91 | 10.984237 | 0.918 | 0.086 | 1.24E−87 | iCN | TAC1 |
| BASP15 | 6.39E−86 | 4.1772011 | 1 | 0.349 | 1.92E−82 | iCN | BASP1 |
| ELAVL25 | 1.09E−82 | 5.5811162 | 0.918 | 0.231 | 3.26E−79 | iCN | ELAVL2 |
| NR2F24 | 1.39E−82 | 4.1473185 | 0.965 | 0.373 | 4.17E−79 | iCN | NR2F2 |
| MEIS23 | 1.47E−80 | 7.9774748 | 0.895 | 0.15 | 4.42E−77 | iCN | MEIS2 |
| NEFM4 | 3.77E−79 | 7.7670574 | 0.889 | 0.176 | 1.13E−75 | iCN | NEFM |
| MAP1B5 | 2.65E−73 | 4.3989468 | 0.994 | 0.348 | 7.96E−70 | ICN | MAP1B |
| RTN14 | 4.66E−73 | 5.1748213 | 0.965 | 0.28 | 1.40E−69 | iCN | RTN1 |
| STMN24 | 3.15E−72 | 10.17605 | 0.988 | 0.297 | 9.46E−69 | iCN | STMN2 |
| LHX9 | 1.04E−24 | 2.5874506 | 0.363 | 0.028 | 3.13E−21 | iCN | LHX9 |
| SCG24 | 0 | 5.6119785 | 0.565 | 0.073 | 0 | immature-iCN | SCG2 |
| BASP16 | 0 | 2.9640136 | 0.931 | 0.319 | 0 | immature-iCN | BASP1 |
| NSG25 | 9.67E−308 | 3.1623774 | 0.679 | 0.152 | 2.90E−304 | immature-iCN | NSG2 |
| STMN25 | 1.64E−306 | 8.5041902 | 0.909 | 0.266 | 4.93E−303 | immature-iCN | STMN2 |
| SCN2A4 | 4.42E−302 | 2.5000461 | 0.444 | 0.072 | 1.33E−298 | immature-iCN | SCN2A |
| CELF45 | 2.54E−300 | 3.7803878 | 0.629 | 0.169 | 7.62E−297 | immature-iCN | CELF4 |
| MAPT5 | 1.70E−296 | 3.0021418 | 0.701 | 0.213 | 5.11E−293 | immature-iCN | MAPT |
| RBFOX14 | 1.59E−292 | 2.2672955 | 0.352 | 0.069 | 4.76E−289 | immature-iCN | RBFOX1 |
| GRIA25 | 3.58E−292 | 2.8067613 | 0.553 | 0.114 | 1.08E−288 | immature-iCN | GRIA2 |
| MEIS24 | 2.40E−231 | 4.8246661 | 0.498 | 0.137 | 7.21E−228 | immature-iCN | MEIS2 |
| STMN26 | 0 | 12.695738 | 0.991 | 0.259 | 0 | GC | STMN2 |
| TUBB37 | 0 | 10.47786 | 0.983 | 0.292 | 0 | GC | TUBB3 |
| GAP436 | 0 | 10.381824 | 0.987 | 0.25 | 0 | GC | GAP43 |
| CAMK2N16 | 0 | 8.5845695 | 0.937 | 0.216 | 0 | GC | CAMK2N1 |
| MAP1B7 | 0 | 5.6437524 | 0.98 | 0.314 | 0 | GC | MAP1B |
| KIF5C8 | 0 | 5.5163498 | 0.962 | 0.279 | 0 | GC | KIF5C |
| PAX63 | 9.24E−273 | 2.9231473 | 0.827 | 0.261 | 2.77E−269 | GC | PAX6 |
| ELAVL27 | 2.34E−272 | 2.784263 | 0.783 | 0.202 | 7.02E−269 | GC | ELAVL2 |
| ERBB44 | 2.51E−271 | 2.5174763 | 0.821 | 0.301 | 7.52E−268 | GC | ERBB4 |
| NEUROD24 | 8.38E−268 | 2.9850148 | 0.687 | 0.088 | 2.52E−264 | GC | NEUROD2 |
| CA81 | 0 | 12.056606 | 0.918 | 0.034 | 0 | PC | CA8 |
| RORA | 0 | 7.3278551 | 0.867 | 0.146 | 0 | PC | RORA |
| RTN17 | 7.83E−285 | 3.6290379 | 0.916 | 0.251 | 2.35E−281 | PC | RTN1 |
| CALB11 | 7.51E−270 | 3.7716164 | 0.681 | 0.154 | 2.25E−266 | PC | CALB1 |
| SKOR21 | 2.12E−256 | 3.7297616 | 0.428 | 0.013 | 6.37E−253 | PC | SKOR2 |
| MAP210 | 1.73E−235 | 2.6105875 | 0.845 | 0.321 | 5.20E−232 | PC | MAP2 |
| RTN410 | 4.07E−211 | 1.8181158 | 0.876 | 0.351 | 1.22E−207 | PC | RTN4 |
| EBF18 | 8.68E−211 | 1.8175889 | 0.471 | 0.131 | 2.60E−207 | PC | EBF1 |
| DAB14 | 8.96E−211 | 2.0964457 | 0.306 | 0.038 | 2.69E−207 | PC | DAB1 |
| PCP4L12 | 1.87E−141 | 1.2476378 | 0.136 | 0.014 | 5.61E−138 | PC | PCP4L1 |
| PTN5 | 0 | 11.150863 | 0.917 | 0.182 | 0 | Glia | PTN |
| FABP74 | 0 | 5.0883918 | 0.782 | 0.164 | 0 | Glia | FABP7 |
| VIM5 | 0 | 4.8603933 | 0.906 | 0.247 | 0 | Glia | VIM |
| C1orf613 | 0 | 4.5331668 | 0.791 | 0.177 | 0 | Glia | C1orf61 |
| GSN4 | 0 | 3.1580657 | 0.832 | 0.222 | 0 | Glia | GSN |
| GPM6B4 | 0 | 3.1137854 | 0.819 | 0.202 | 0 | Glia | GPM6B |
| ZFP36L15 | 0 | 2.3763269 | 0.8 | 0.286 | 0 | Glia | ZFP36L1 |
| ID14 | 2.26E−280 | 3.1708653 | 0.719 | 0.277 | 6.79E−277 | Glia | ID1 |
| IRX35 | 2.78E−269 | 2.0469302 | 0.553 | 0.124 | 8.34E−266 | Glia | IRX3 |
| TTYH14 | 1.31E−246 | 2.2021335 | 0.607 | 0.178 | 3.92E−243 | Glia | TTYH1 |
| IGFBPL13 | 0 | 7.6979844 | 0.978 | 0.165 | 0 | GCP | IGFBPL1 |
| SFRP13 | 0 | 7.6520701 | 0.899 | 0.159 | 0 | GCP | SFRP1 |
| SH3BGRL33 | 0 | 4.9890308 | 0.861 | 0.248 | 0 | GCP | SH3BGRL3 |
| SSTR24 | 0 | 3.3986179 | 0.81 | 0.16 | 0 | GCP | SSTR2 |
| NFIB7 | 0 | 3.3292357 | 0.887 | 0.245 | 0 | GCP | NFIB |
| INSM14 | 0 | 3.0047062 | 0.737 | 0.12 | 0 | GCP | INSM1 |
| NRN15 | 0 | 2.9427184 | 0.807 | 0.201 | 0 | GCP | NRN1 |
| PTMA9 | 0 | 2.8548073 | 0.973 | 0.337 | 0 | GCP | PTMA |
| BARHL13 | 0 | 2.8009578 | 0.656 | 0.079 | 0 | GCP | BARHL1 |
| AKAP69 | 0 | 2.5858939 | 0.758 | 0.207 | 0 | GCP | AKAP6 |
| CYP1B13 | 0 | 3.7368907 | 0.544 | 0.123 | 0 | Progenitors | CYP1B1 |
| RBP13 | 0 | 3.6331416 | 0.727 | 0.186 | 0 | Progenitors | RBP1 |
| TRPM32 | 0 | 3.1876048 | 0.737 | 0.203 | 0 | Progenitors | TRPM3 |
| MSX12 | 0 | 2.8412866 | 0.733 | 0.212 | 0 | Progenitors | MSX1 |
| SPARCL12 | 0 | 2.4778985 | 0.454 | 0.155 | 0 | Progenitors | SPARCLI |
| ZFP36L22 | 0 | 2.3033103 | 0.737 | 0.211 | 0 | Progenitors | ZFP36L2 |
| HES14 | 0 | 2.1621368 | 0.749 | 0.266 | 0 | Progenitors | HES1 |
| KRT182 | 0 | 2.1481491 | 0.759 | 0.188 | 0 | Progenitors | KRT18 |
| FOS3 | 0 | 2.0804045 | 0.672 | 0.285 | 0 | Progenitors | FOS |
| CA22 | 0 | 2.0352457 | 0.571 | 0.17 | 0 | Progenitors | CA2 |

TABLE 6
Top 25 differentially expressed genes (DEGs) in each of SVZ,
VZ, and IZ clusters of the cerebellum-like organoid cells.

| | p_val | avg_diff | pct. 1 | pct. 2 | p_val_adj | cluster | gene |
|---|---|---|---|---|---|---|---|
| TFAP2A | 4.90E−13 | 3.20323344 | 0.742 | 0.055 | 1.62E−09 | IZ | TFAP2A |
| BTG1 | 4.20E−12 | 2.6973752 | 0.903 | 0.367 | 1.39E−08 | IZ | BTG1 |
| RGS16 | 4.35E−12 | 3.39382904 | 0.806 | 0.072 | 1.44E−08 | IZ | RGS16 |
| ASCL1 | 1.55E−11 | 3.1663764 | 0.839 | 0.122 | 5.12E−08 | IZ | ASCL1 |
| TAGLN3 | 1.39E−10 | 2.20222787 | 0.871 | 0.393 | 4.61E−07 | IZ | TAGLN3 |
| SOX4 | 1.87E−10 | 2.56886415 | 0.935 | 0.367 | 6.19E−07 | IZ | SOX4 |
| DCLK2 | 2.19E−10 | 2.08699623 | 0.839 | 0.305 | 7.24E−07 | IZ | DCLK2 |
| MIAT | 4.34E−10 | 2.14981443 | 0.839 | 0.272 | 1.44E−06 | IZ | MIAT |
| CYB5A | 1.19E−09 | 1.97360014 | 0.806 | 0.372 | 3.94E−06 | IZ | CYB5A |
| DPYSL3 | 3.07E−09 | 1.79193646 | 0.839 | 0.39 | 1.02E−05 | IZ | DPYSL3 |
| ARHGAP21 | 3.46E−09 | 1.52540317 | 0.806 | 0.383 | 1.15E−05 | IZ | ARHGAP21 |
| TFAP2B | 1.42E−08 | 3.33053512 | 0.71 | 0.017 | 4.70E−05 | IZ | TFAP2B |
| PHLDA1 | 2.95E−08 | 2.05235237 | 0.677 | 0.171 | 9.75E−05 | IZ | PHLDA1 |
| PRDM13 | 3.80E−08 | 2.75726843 | 0.613 | 0.026 | 0.00012593 | IZ | PRDM13 |
| POU3F2 | 6.53E−08 | 2.41881608 | 0.71 | 0.205 | 0.00021618 | IZ | POU3F2 |
| GAD2 | 6.53E−08 | 2.06759528 | 0.452 | 0.012 | 0.00021618 | IZ | GAD2 |
| KIF21A | 1.25E−07 | 1.36105334 | 0.774 | 0.362 | 0.00041235 | IZ | KIF21A |
| MARCKS | 1.33E−07 | 1.39506463 | 0.839 | 0.422 | 0.00044154 | IZ | MARCKS |
| GPM6A | 1.46E−07 | 1.33188572 | 0.774 | 0.378 | 0.00048359 | IZ | GPM6A |
| LHX1 | 3.06E−07 | 1.91524686 | 0.452 | 0.028 | 0.00101367 | IZ | LHX1 |
| PPP2R2B | 3.33E−07 | 1.05494055 | 0.355 | 0.057 | 0.00110128 | IZ | PPP2R2B |
| ROBO1 | 4.62E−07 | 1.58047855 | 0.645 | 0.247 | 0.00153096 | IZ | ROBO1 |
| SRGAP3 | 4.88E−07 | 1.30777446 | 0.645 | 0.378 | 0.00161675 | IZ | SRGAP3 |
| PLXNA2 | 8.11E−07 | 1.50735527 | 0.548 | 0.145 | 0.00268535 | IZ | PLXNA2 |
| ZDHHC22 | 9.67E−07 | 1.50896861 | 0.355 | 0.021 | 0.00320186 | IZ | ZDHHC22 |
| PTN | 5.20E−82 | 4.89287813 | 0.881 | 0.05 | 1.72E−78 | VZ | PTN |
| ID1 | 8.83E−64 | 3.11363234 | 0.773 | 0.123 | 2.92E−60 | VZ | ID1 |
| TTYH1 | 3.22E−63 | 2.88979039 | 0.71 | 0.032 | 1.07E−59 | VZ | TTYH1 |
| HES4 | 2.30E−56 | 2.69342174 | 0.717 | 0.102 | 7.60E−53 | VZ | HES4 |
| GPM6B | 2.00E−53 | 2.19592218 | 0.688 | 0.117 | 6.62E−50 | VZ | GPM6B |
| ID3 | 1.01E−52 | 2.25407708 | 0.643 | 0.061 | 3.35E−49 | VZ | ID3 |
| FABP7 | 3.20E−51 | 2.34638359 | 0.669 | 0.085 | 1.06E−47 | VZ | FABP7 |
| ID2 | 1.18E−49 | 2.02154122 | 0.647 | 0.123 | 3.91E−46 | VZ | ID2 |
| NTRK2 | 1.19E−49 | 1.91130895 | 0.658 | 0.094 | 3.93E−46 | VZ | NTRK2 |
| VIM | 2.89E−47 | 2.14079109 | 0.751 | 0.193 | 9.58E−44 | VZ | VIM |
| ATP1A2 | 2.44E−46 | 1.7685606 | 0.632 | 0.035 | 8.08E−43 | VZ | ATP1A2 |
| SPARC | 2.66E−44 | 1.60608437 | 0.654 | 0.132 | 8.82E−41 | VZ | SPARC |
| LY6H | 1.13E−40 | 1.46654633 | 0.602 | 0.123 | 3.75E−37 | VZ | LY6H |
| ID4 | 2.45E−39 | 1.72619609 | 0.643 | 0.158 | 8.10E−36 | VZ | ID4 |
| GSN | 2.92E−38 | 1.82896638 | 0.651 | 0.143 | 9.67E−35 | VZ | GSN |
| WLS | 1.62E−37 | 1.41580474 | 0.643 | 0.152 | 5.37E−34 | VZ | WLS |
| IGFBP2 | 6.76E−37 | 1.41294774 | 0.662 | 0.219 | 2.24E−33 | VZ | IGFBP2 |
| C1orf611 | 1.95E−36 | 1.94558984 | 0.669 | 0.184 | 6.44E−33 | VZ | C1orf61 |
| NES | 4.38E−35 | 1.52110069 | 0.569 | 0.132 | 1.45E−31 | VZ | NES |
| CLU | 2.35E−34 | 1.62283122 | 0.558 | 0.091 | 7.78E−31 | VZ | CLU |
| ZFP36L1 | 3.00E−34 | 1.51708688 | 0.658 | 0.234 | 9.93E−31 | VZ | ZFP36L1 |
| CNTNAP2 | 1.70E−33 | 1.67665728 | 0.599 | 0.14 | 5.64E−30 | VZ | CNTNAP2 |
| EGR1 | 2.38E−33 | 1.76716083 | 0.639 | 0.143 | 7.89E−30 | VZ | EGR1 |
| PEA15 | 6.35E−33 | 1.51039606 | 0.613 | 0.161 | 2.10E−29 | VZ | PEA15 |
| MGST1 | 6.61E−33 | 1.3506324 | 0.569 | 0.181 | 2.19E−29 | VZ | MGST1 |
| IGFBPL1 | 5.15E−87 | 3.4617449 | 0.842 | 0.067 | 1.71E−83 | SVZ | IGFBPL1 |
| CALM1 | 8.93E−71 | 2.8846454 | 0.717 | 0.053 | 2.96E−67 | SVZ | CALM1 |
| NRN1 | 6.72E−67 | 2.12920965 | 0.646 | 0.063 | 2.23E−63 | SVZ | NRN1 |
| TMSB4X | 7.54E−63 | 3.13943726 | 0.765 | 0.12 | 2.50E−59 | SVZ | TMSB4X |
| PTMA | 1.73E−60 | 1.95190561 | 0.791 | 0.123 | 5.73E−57 | SVZ | PTMA |
| NPTX2 | 1.95E−60 | 2.14625518 | 0.656 | 0.06 | 6.46E−57 | SVZ | NPTX2 |
| HES6 | 3.23E−57 | 3.08197752 | 0.701 | 0.143 | 1.07E−53 | SVZ | HES6 |
| TUBB31 | 8.77E−56 | 2.30083767 | 0.691 | 0.143 | 2.91E−52 | SVZ | TUBB3 |
| SSTR2 | 8.30E−51 | 1.80028214 | 0.617 | 0.063 | 2.75E−47 | SVZ | SSTR2 |
| INSM11 | 3.68E−46 | 1.77577301 | 0.614 | 0.09 | 1.22E−42 | SVZ | INSM1 |
| SFRP1 | 4.28E−40 | 2.06383273 | 0.643 | 0.193 | 1.42E−36 | SVZ | SFRP1 |
| TUBA1A | 1.14E−39 | 1.5827551 | 0.666 | 0.233 | 3.76E−36 | SVZ | TUBA1A |
| BARHL1 | 9.63E−39 | 1.48250375 | 0.605 | 0.027 | 3.19E−35 | SVZ | BARHL1 |
| GNG4 | 1.49E−37 | 1.26699819 | 0.643 | 0.177 | 4.93E−34 | SVZ | GNG4 |
| SOX41 | 3.65E−37 | 1.65492814 | 0.614 | 0.17 | 1.21E−33 | SVZ | SOX4 |
| CAMK2N1 | 5.28E−37 | 2.02896779 | 0.582 | 0.09 | 1.75E−33 | SVZ | CAMK2N1 |
| CMIP | 2.20E−35 | 1.41180739 | 0.569 | 0.123 | 7.30E−32 | SVZ | CMIP |
| SH3BGRL3 | 5.93E−34 | 1.6222869 | 0.598 | 0.14 | 1.96E−30 | SVZ | SH3BGRL3 |
| GAP43 | 7.23E−32 | 2.02284184 | 0.559 | 0.123 | 2.39E−28 | SVZ | GAP43 |
| SOX111 | 8.60E−32 | 1.30727406 | 0.592 | 0.177 | 2.85E−28 | SVZ | SOX11 |
| STMN2 | 3.55E−30 | 2.13324199 | 0.569 | 0.087 | 1.18E−26 | SVZ | STMN2 |
| BASP1 | 3.70E−30 | 1.17961068 | 0.605 | 0.22 | 1.23E−26 | SVZ | BASP1 |
| ACTB | 1.04E−29 | 0.97504624 | 0.643 | 0.267 | 3.43E−26 | SVZ | ACTB |
| RHOBTB3 | 5.93E−29 | 1.0176752 | 0.608 | 0.217 | 1.97E−25 | SVZ | RHOBTB3 |
| AKAP6 | 2.78E−28 | 1.15459931 | 0.559 | 0.187 | 9.20E−25 | SVZ | AKAP6 |

TABLE 7
Purkinje cell vs other cell type enrichment test statistics.

| name | pval | adj_pval | f_statistic | df1 | df2 | lfc | contrast |
|---|---|---|---|---|---|---|---|
| CA8 | 1.74E−16 | 4.70E−15 | 171.4205 | 1 | 42.36348 | 10 | PC - non-PC |
| PCP2 | 1.38E−09 | 1.87E−08 | 59.32695 | 1 | 42.36348 | 7.167861 | PC - non-PC |
| EBF1 | 3.19E−08 | 2.87E−07 | 45.55415 | 1 | 42.36348 | 5.839895 | PC - non-PC |
| DAB1 | 8.92E−08 | 4.82E−07 | 41.46936 | 1 | 42.36348 | 5.472301 | PC - non-PC |
| CNTNAP4 | 8.14E−08 | 4.82E−07 | 41.82505 | 1 | 42.36348 | 5.452931 | PC - non-PC |
| EBF3 | 3.16E−07 | 1.42E−06 | 36.72171 | 1 | 42.36348 | 5.035436 | PC - non-PC |
| ITPR1 | 1.74E−06 | 6.30E−06 | 30.75005 | 1 | 42.36348 | 4.676643 | PC - non-PC |
| NRGN | 1.87E−06 | 6.30E−06 | 30.51706 | 1 | 42.36348 | 4.551813 | PC - non-PC |
| CALB1 | 1.37E−05 | 4.10E−05 | 24.17904 | 1 | 42.36348 | 3.951149 | PC - non-PC |
| FOXP2 | 7.19E−05 | 1.94E−04 | 19.35343 | 1 | 42.36348 | 3.401057 | PC - non-PC |
| RORA | 6.23E−04 | 0.001529 | 13.66304 | 1 | 42.36348 | 2.778938 | PC - non-PC |
| EBF2 | 0.003958 | 0.008905 | 9.290797 | 1 | 42.36348 | 2.357294 | PC - non-PC |
| EN1 | 0.011046 | 0.022942 | 7.064287 | 1 | 42.36348 | 1.98281 | PC - non-PC |
| RORB | 0.022584 | 0.043555 | 5.601961 | 1 | 42.36348 | 1.943956 | PC - non-PC |
| PAX2 | 0.066964 | 0.120536 | 3.535261 | 1 | 42.36348 | 1.384255 | PC - non-PC |
| LUM | 0.091163 | 0.144789 | 2.988066 | 1 | 42.36348 | 1.354666 | PC - non-PC |
| CDH4 | 0.076969 | 0.129885 | 3.285871 | 1 | 42.36348 | 1.308372 | PC - non-PC |
| EEF2 | 0.186159 | 0.279238 | 1.805862 | 1 | 42.36348 | 0.945369 | PC - non-PC |
| MEIS2 | 0.47207 | 0.670837 | 0.526503 | 1 | 42.36348 | 0.562282 | PC - non-PC |
| FOXP1 | 0.580652 | 0.712619 | 0.30993 | 1 | 42.36348 | 0.40963 | PC - non-PC |
| BARHL1 | 0.641939 | 0.75358 | 0.219347 | 1 | 42.36348 | 0.372079 | PC - non-PC |
| WLS | 0.702422 | 0.790224 | 0.147955 | 1 | 42.36348 | 0.271423 | PC - non-PC |
| ETV1 | 0.776971 | 0.806855 | 0.081271 | 1 | 42.36348 | 0.215813 | PC - non-PC |
| GFAP | 0.871045 | 0.871045 | 0.026673 | 1 | 42.36348 | −0.11476 | PC - non-PC |
| CDH9 | 0.746362 | 0.806071 | 0.105987 | 1 | 42.36348 | −0.37678 | PC - non-PC |
| CDH10 | 0.560407 | 0.712619 | 0.344415 | 1 | 42.36348 | −0.44611 | PC - non-PC |
| TTR | 0.57041 | 0.712619 | 0.327074 | 1 | 42.36348 | −0.49774 | PC - non-PC |

| name | l2fc | Name | pval.signif | adj_pval.signif |
|---|---|---|---|---|
| CA8 | lnf | CA8 * | **** | **** |
| PCP2 | 6.733349 | PCP2 * | **** | **** |
| EBF1 | 6.106469 | EBF1 * | **** | **** |
| DAB1 | 5.696526 | DAB1 * | **** | **** |
| CNTNAP4 | 5.531944 | CNTNAP4 * | **** | **** |
| EBF3 | 5.24813 | EBF3 * | **** | **** |
| ITPR1 | 4.583811 | ITPR1 * | **** | **** |
| NRGN | 4.892485 | NRGN | **** | **** |
| CALB1 | 4.440781 | CALB1 * | **** | **** |
| FOXP2 | 3.626777 | FOXP2 * | **** | **** |
| RORA | 3.046389 | RORA * | *** | ** |
| EBF2 | 2.175017 | EBF2 * | ** | ** |
| EN1 | 2.068635 | EN1 | * | * |
| RORB | 1.473658 | RORB | * | * |
| PAX2 | 1.030347 | PAX2 | ns | ns |
| LUM | 0.986749 | LUM | ns | ns |
| CDH4 | 1.079223 | CDH4 | ns | ns |
| EEF2 | 0.913739 | EEF2 | ns | ns |
| MEIS2 | 0.010611 | MEIS2 | ns | ns |
| FOXP1 | 0.042143 | FOXP1 * | ns | ns |
| BARHL1 | −0.03395 | BARHL1 | ns | ns |
| WLS | 0.121441 | WLS | ns | ns |
| ETV1 | −0.2127 | ETV1 | ns | ns |
| GFAP | −0.1855 | GFAP | ns | ns |
| CDH9 | −0.09764 | CDH9 * | ns | ns |
| CDH10 | −0.96004 | CDH10 | ns | ns |
| TTR | −056039 | TTR | ns | ns |

TABLE 8
Passive properties of the membrane for PCP2+
neurons recorded by whole-cell patch-clamp recordings.

| | | | | | | Resting |
|---|---|---|---|---|---|---|
| | | | Membrane | Membrane | Input | Membrane |
| | | | Capacitance | Resistance | Resistance | Potential |
| DIV | BD | Cell line | (pF) | (MΩ) | (MΩ) | (mV) |
| 146 | 80522 | 11a | 78.67 | 566.7 | 63.4 | −42 |
| 147 | 80522 | 11a | 79.63 | 95.7 | 54.6 | −75.4 |
| 147 | 80522 | 11a | 120.54 | 473.5 | 23 | −68.1 |
| 229 | 52722 | pgpfoxg1 | 87.97 | 133.9 | 28.3 | −69.3 |
| 137 | 100722 | 11a | 1.9 | 1200 | 581 | −46 |
| 137 | 100722 | 11a | 23.63 | 1900 | 143.6 | −47.6 |
| 138 | 100722 | 11a | 38.64 | 2600 | 95 | −43.7 |
| 138 | 100722 | 11a | 22.43 | 1100 | 538.9 | −40.7 |
| 138 | 100722 | 1la | 26.49 | 8100 | 101.4 | −42.9 |
| 147 | 100722 | 11a | 31.35 | 1100 | 24.5 | −53.5 |
| 147 | 100722 | 11a | 122.92 | 115.8 | 35.8 | −68 |
| 147 | 100722 | 11a | 39.34 | 1200 | 19.4 | −47 |
| 147 | 100722 | 11a | 37.33 | 723.7 | 138.6 | −54.4 |
| 147 | 100722 | 11a | 145.56 | 485.6 | 226.8 | −57 |
| 149 | 100722 | 11a | 87.77 | 671.4 | 108.8 | −55.1 |
| 149 | 100722 | 11a | 70.39 | 1200 | 106.7 | −51 |
| 149 | 100722 | 11a | 25.7 | 1600 | 29.7 | −40 |
| 149 | 100722 | 1la | 47.29 | 637.4 | 74.9 | −69 |
| 149 | 100722 | 11a | 58.53 | 1600 | 43.5 | −53.1 |
| | | AVG | 60.32 | 1342.3 | 128.3105263 | −53.88421053 |

TABLE 9
Human primer sequence for quantitative real time PCR.

| Gene | | |
|---|---|---|
| Name | Forward Primer (5′-3′) | Reverse Primer (5′-3′) |
| EN1 | GAGCGCAGGGCACCAAATA (SEQ | CGAGTCAGTTTTGACCACGG (SEQ |
| | ID NO: 1) | ID NO: 2) |
| EN2 | CCGGCGTGGGTCTACTGTA (SEQ | GGCCGCTTGTCCTCTTTGTT (SEQ |
| | ID NO: 3) | ID NO: 4) |
| FOXG1 | CCTGCCCTGTGAGTCTTTAAG | GTTCACTTACAGTCTGGTCCC |
| | (SEQ ID NO: 5) | (SEQ ID NO: 6) |
| GBX2 | GACGAGTCAAAGGTGGAAGAC | GATTGTCATCCGAGCTGTAGTC |
| | (SEQ ID NO: 7) | (SEQ ID NO: 8) |
| HOXA2 | CGTCGCTCGCTGAGTGCCTG (SEQ | TGTCGAGTGTGAAAGCGTCGAGG |
| | ID NO: 9) | (SEQ ID NO: 10) |
| HOXA5 | TCATAGTTCCGTGAGCGAGC | ATCCATGCCATTGTAGCCGT (SEQ |
| | (SEQ ID NO: 11) | ID NO: 12) |
| IRX3 | GGCTTGCGCCCCGTAGAAATGT | AGGAGCCAGGTCAGGTCCGAAC |
| | (SEQ ID NO: 13) | (SEQ ID NO: 14) |
| OTX2 | AGAGGACGACGTTCACTCG (SEQ | TCGGGCAAGTTGATTTTCAGT (SEQ |
| | ID NO: 15) | ID NO: 16) |
| PAX8 | ATAGCTGCCGACTAAGCATTGA | ATCCGTGCGAAGGTGCTTT (SEQ ID |
| | (SEQ ID NO: 17) | NO: 18) |
| SIX3 | ACCGGCCTCACTCCCACACA | CGCTCGGTCCAATGGCCTGG (SEQ |
| | (SEQ ID NO: 19) | ID NO: 20) |

TABLE 10
Exemplary antibodies for tissue characterization

| | | | Catalog | | Temporal |
|---|---|---|---|---|---|
| Cell Type/tissue | Antigen | Supplier | Number | Dilution | Expression |
| Rhombic Lip | Rabbit monoclonal | Abclonal | A11A477 | 1:200 | From Day 30 |
| progenitors | anti-ATOH1 | | | | |
| Rhombic Lip | Rabbit polyclonal | Atlas | HPA004809 | 1:500 | From Day 30 |
| derived Excitatory | anti-BARHL1 | Antibodies | | | |
| neuronal precursors | | | | | |
| Ventricular Zone | Rabbit polyclonal | Proteintech | 10890-1-AP | 1:100 | From Day 30 |
| progenitors | anti-KIRREL2 | | | | |
| Ventricular Zone | Rabbit polyclonal | Atlas | HPA046206 | 1:500 | From Day 30 |
| derived postmitotic | anti-SKOR2 | Antibodies | | | |
| Purkinje cell | | | | | |
| precursors | | | | | |
| Granule Cells | Rabbit polyclonal | Biolegend | 901301 | 1:200 | From Day 30 |
| | anti-PAX6 | | | | |
| | TBR2 | | | | From Day 60 |
| Purkinje Cells | Mouse monoclonal | Sigma- | C9848- | 1:300 | From Day 60 |
| | anti-CALB1 | Aldrich | 100UL | | |
| Radial Glia | Goat polyclonal | RD systems | AF2018 | 1:500 | From Day 30 |
| | anti-SOX2 | | | | |
| Rostral | Rabbit XX OTX2 | | | 1:200 | ~Day 16 |
| (forebrain/midbrain) | | | | | |
| neural tube marker | | | | | |
| Caudal (hindbrain) | Goat XXX GBX2 | | | 1:200 | ~Day 16 |
| neural tube marker | | | | | |
| | MAP2 | | | | From Day 30 |
| | GABA | | | | From Day 60 |
| | NEUN | | | | From Day 60 |
| | GFAP | | | | From Day 60 |

TABLE 11
Growth media and supplements

| | | |
|---|---|---|
| AmphoB | Gibco | 15290018 |
| Apo-transferrin | Sigma | T1147-100MG |
| BONE | R&D Systems | 248-BDB-050 |
| BrainPhys Optimized Medium | STEMCELL | 05796 |
| | Technologies | |
| Bovine Serum Abumin (BSA) (fatty | Sigma | A0281 |
| acid free and globulin free) | | |
| BSA (for all else) | | |
| B-27 + vitA supplement | Gibco | 17504001 |
| chemically defined lipid concentrate | Gibco | 11905031 |
| (CDLC) | | |
| CHIR | Reprocell | 04-0004 |
| DMEM/F-12 | Corning/Cytiva | SH30023.FS |
| FGF8b | Peprotech | 100-25 |
| Glutamax | Gibco | 35050061 |
| Ham's F-12 medium | Gibco | 11-765-054 |
| Heparin | Sigma | H314-25KU |
| IMDM medium | Gibco | 12440053 |
| Insulin | Sigma | 19278-5ML |
| Knockout Serum Replacement (KSR) | Gibco | 10828028 |
| Mono-Thioglycerol | Sigma | M1753 |
| mTeSR1medium | Stem Cell | 4309155 |
| | Technologies | |
| 5X supplement | | |
| Neurobasal medium | Gibco | 21103049 |
| NeuroCult SM1 Neuronal Supplement | STEMCELL | 05711 |
| | Technologies | |
| NMDA | Tocris | 0114 |
| Noggin | Peprotech | 120-10C |
| N2 supplement | Gibco | 17502001 |
| ROCK inhibitor | STEMCELL | 72302 |
| | Technologies | |
| SB431542 (TGFBi) | Selleckchem | S1067 |
| SDF1a | Peprotech | 300-28A |
| T3 | Sigma | T2877-250MG |
| 2-Mercaptoethanol | Gibco | 21985023 |

TABLE 12
Enzyme and other reagents

| | | |
|---|---|---|
| Accutase | Sigma | 07922 |
| DMSO | | |
| Fluoromount | EMS | 50-259-73 |
| Geltrex | Thermo Fisher Scientific | A1413301 |
| Growth-factor reduced Matrigel | Corning | 354230 |
| Trypan blue | | |
| Papain Dissociation solution | | |
| Paraformaldehyde | | |
| Penicillin/streptomycin | Corning | SV30010 |
| PTX | Hello Bio | HB0506 |
| Sucrose | | |
| SYBR Green PCR Master Mx | Thermo Fisher Scientific | 4309155 |
| Tetrodotoxin, TTX | Tocris | 1078 |
| Tissue-Tek O.C.T. compound | Sakura | 62550 |
| TritonX-100 | Sigma | T9284 |
| Tween20 | Sigma | P9416 |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

What is claimed is:

1. A method of generating a cerebellum-like organoid from human stem cells, comprising:
a. culturing the human stem cells in the presence of two or more SMAD signaling inhibitors, a glycogen synthase kinase 3 (GSK3) inhibitor, and optionally a rho-associated, coiled-coil containing protein kinase (ROCK) inhibitor (ROCKi) in a growth factor-reduced medium to obtain neuronal lineage embryoid bodies (EBs);
b. culturing the neuronal lineage EBs derived from step a) in the presence of a midbrain-hindbrain morphogen and the two or more SMAD signaling inhibitors and the GSK3 inhibitor in the growth factor-reduced medium, thereby obtaining midbrain-hindbrain regionalized tissues;
c. culturing the midbrain-hindbrain regionalized tissues from step b) in the presence of the midbrain-hindbrain morphogen, the two or more SMAD signaling inhibitors, and the GSK3 inhibitor in a first cerebellar differentiation medium (CerDM1), thereby obtaining isthmic organizer regionalized tissues;
d. culturing the isthmic organizer regionalized tissues from step c) in a second cerebellar differentiation medium (CerDM2) in motion, in an agitated environment, or on an orbital shaker, thereby obtaining cerebellar neuroepithelial tissues, wherein the CerDM2 does not contain the one or more SMAD signaling inhibitors, the GSK3 inhibitor, the ROCKi, and the midbrain-hindbrain morphogen; and
e. culturing the cerebellar neuroepithelial tissues from step d) in the presence of a thyroid hormone and optionally further in presence of one or more of a solubilized basement membrane matrix, stromal cell-derived factor 1 alpha (SDF1a), and brain-derived neurotrophic factor (BDNF) in a third cerebellar differentiation medium (CerDM3) thereby forming a cerebellum-like organoid,
wherein the cerebellum-like organoids contain mature, functional Purkinje cells.

2. The method of claim 1, wherein the mature, functional Purkinje cells are positive for PCP2, DAB1, RORA, and FOXP2 and characterized with hyperpolarization-activated current and repetitive spontaneous firing, and wherein the method does not include culturing in the presence of mouse granule cells or mouse glial cells.

3. The method of claim 1, wherein the two or more SMAD signaling inhibitors comprise SB431542 and noggin, the GSK3 inhibitor comprises CHIR99021, the ROCKi comprises Y-27632, the midbrain-hindbrain morphogen comprises fibroblast growth factor 8b (FGF8b), and the thyroid hormone comprises T3.

4. The method of claim 3, wherein step c) comprises culturing in the presence of a first concentration of the FGF8b, the SB431542 and the noggin, and the CHIR99021 in the CerDM1 for a first period of time, followed by culturing in the presence of a second concentration of the FGF8b, the SB431542 and the noggin, and the CHIR99021 in the CerDM1 for a second period of time, wherein the second concentration of the FGF8b is higher than the first concentration of the FGF8b.

5. The method of claim 4, wherein the first concentration of the FGF8b in the CerDM1 is about 100 ng/mL, and the second concentration of the FGF8b in the CerDM1 is about 300 ng/mL.

6. The method of claim 1, wherein the growth factor-reduced medium is a growth factor-reduced chemically-defined medium (gfCDM) comprising a mixture of Iscove's Modified Dulbecco's Medium (IMDM) and Ham's F-12 nutrient mix (F-12), supplemented with bovine serum albumin, a chemically defined lipid concentrate (CDLC), apo-transferrin, mono-thioglycerol, and insulin, and lacking a growth factor;
wherein the CerDM1 comprises a mixture of Dulbecco's Modified Eagle Medium (DMEM) and F-12, supplemented with knockout serum replacement (KSR), apo-transferrin, insulin, glutamax (L-alanyl-L-glutamine dipeptide), and 2-mercaptoethanol;
wherein the CerDM2 comprises a mixture of DMEM and F-12, supplemented with N-2 supplement, B-27, and glutamax (L-alanyl-L-glutamine dipeptide); and
wherein the CerDM3 comprises a mixture of DMEM, F-12, and neurobasal medium, supplemented with N-2 supplement, B-27, glutamax (L-alanyl-L-glutamine dipeptide), heparin, CDLC, and amphotericin B.

7. The method of claim 1, wherein the culturing in step b) comprises culturing for about 4 days or between 3 and 6 days;
wherein the culturing in step c) comprises culturing for about 11 days or between 9 and 13 days;
wherein the culturing in step d) comprises culturing for about 13 days or between 10 and 15 days; and
wherein the culturing in step e) comprises culturing for about 30 days or between 20 and 40 days in the presence of the thyroid hormone, the solubilized basement membrane matrix, and the SDF1a, and optionally further comprising subsequent culturing in the presence of the BDNF after the about 30 days or the between 20 and 40 days.

8. The method of claim 1, wherein the cerebellum-like organoids contains KIRREL2+ cells, ATOH1+ cells, BARHL1+ granule cell progenitors, and SKOR2+ Purkinje neurons, wherein the KIRREL2+ cells and the ATOH1+ cells are spatially segregated.

9. The method of claim 1, wherein the cerebellum-like organoids each contain ventricular zone progenitor cells, rhombic lip progenitor cells, a cluster of Bergmann glial marker-positive cells, a cluster of neuronal cells, a cluster of interneuron precursors, a cluster of interneurons, a cluster of cerebellar nuclei, and glutamatergic neurons;
the cerebellum-like organoids further comprising:
choroid plexus or TTR+ cells,
meninges or LUM+DCN+ cells, and
roof plate cells or GDF7+ cells;
and
wherein the ventricular zone progenitors are positive for KIRREL2, PTF1A, and VIM,
the rhombic lip progenitors are positive for ATOH1 and BARHL1,
the Bergmann glial marker-positive cells express or are positive for PTPRZ1, EDNRB, GFAP, HOPX, SLCA4A4, and EDNRB,
the cluster of neuronal cells comprises the cells expressing the Purkinje cell markers, the Purkinje cell markers comprising SKOR2, RORA, FOXP2, and CALB1,
the cluster of interneuron precursor cells express or are positive for PAX2,
the cluster of interneurons express or are positive for SOX14 and DMBX1,
the cluster of cerebellar nuclei express or are positive for MEIS2, LHX9, and IRX3, and
the glutamatergic neurons express or are positive for STMN2 and SLC17A7, wherein the glutamatergic neurons comprise unipolar brush cells (UBC), granule cells (GC), and granule cell progenitors (GCP); or the glutamaterigic neurons comprise a cluster of cells expressing or positive for EOMES, a cluster of cells expressing or positive for NEUROD1 and NHLH1, and a cluster of cells expressing or positive for ATOH1 and BARHL1.

10. The method of claim 1, wherein the human stem cells comprise human induced pluripotent stem cells.

11. The method of claim 1, wherein the human stem cells comprise human embryonic stem cells.

12. A cerebellum-like organoid generated from the method of claim 2.

13. A cerebellum-like organoid based solely on human cells, wherein the cerebellum-like organoid contains spatially segregated ventricular zone progenitors and rhombic lip progenitors, and the cerebellum-like organoid contains cells expressing Purkinje cell markers.

14. The cerebellum-like organoid of claim 13, wherein the Purkinje cell markers comprise (i) SKOR2, RORA, FOXP2, and CALB1, or (ii) PCP2, DAB1, RORA, and FOXP2.

15. The cerebellum-like organoid of claim 13, wherein the cerebellum-like organoid further comprises a cluster of Bergmann glial marker-positive cells, a cluster of neuronal cells, a cluster of interneuron precursors, a cluster of interneurons, a cluster of cerebellar nuclei, and glutamatergic neurons;
the cerebellum-like organoid further comprising:
choroid plexus or TTR+ cells,
meninges or LUM+DCN+ cells, and
roof plate cells or GDF7+ cells;
and
wherein the ventricular zone progenitors are KIRREL2+, PTF1A+, and VIM+,
the rhombic lip progenitors are ATOH1+ and BARHL1+,
the Bergmann glial marker-positive cells express or are positive for PTPRZ1, EDNRB, GFAP, HOPX, SLCA4A4, and EDNRB,
the cluster of neuronal cells comprises the cells expressing the Purkinje cell markers,
the cluster of interneuron precursors express or are positive for PAX2,
the cluster of interneurons express or are positive for SOX14 and DMBX1,
the cluster of cerebellar nuclei express or are positive for MEIS2, LHX9, and IRX3, and
the glutamatergic neurons express or are positive for STMN2+ and SLC17A7+, wherein the glutamatergic neurons comprise unipolar brush cells (UBC), granule cells (GC), and granule cell progenitors (GCP); or the glutamaterigic neurons comprise a cluster of cells expressing or positive for EOMES, a cluster of cells expressing or positive for NEUROD1 and NHLH1, and a cluster of cells expressing or positive for ATOH1 and BARHL1.

16. A method of screening a therapeutic agent, the method comprising:
contacting cell cultures in step b), c), d), and/or e) of the cerebellum-like organoid generation method of claim 1 with the therapeutic agent, and
detecting an alteration in the cerebellum-like organoid in response to the therapeutic agent.

17. The method of claim 16, wherein the alteration is an alteration in viability and/or activity of the cerebellum-like organoid compared to viability and/or activity of an untreated control cerebellum-like organoid; or an alteration in the expression of a neural marker compared to the expression of the neural marker of an untreated control cerebellum-like organoid.

18. A method of screening a candidate drug, comprising:
contacting a cerebellum-like organoid of claim 12 with a candidate drug, and
assaying survival, activity, and/or expression of a neural marker of the cerebellum-like organoid of claim 12 in response to the candidate drug.

19. The method of claim 18, wherein cerebellum-like organoid comprises a genetic alteration associated with a neurological disease or condition.

20. The method of claim 19, wherein the genetic alteration is in a polynucleotide of SOX5, SATB2, and/or KCNQ3 in excitatory granule cells of the cerebellum-like organoid, or in a polynucleotide of EHMT1, ZC4H2, PPKAR1A, PPP1CB, QRICH1, KCNH1, U2AF2, NALCN, GNAI1, KCNB1, CHD2, CLTC, NSD1, CYP27C1, and/or GOLPH3 in molecular layer interneurons (MLI) of the cerebellum-like organoid, and the neurological disease or condition comprises intellectual disability;
wherein the genetic alteration is in a polynucleotide of TNRC6B, SMARCC2, FAM98C, CHD2, UBN2, DSCAM, TBL1XR1, and/or CUL3 in the MLI, or in a polynucleotide of USP45, ERBIN, and/or PYHIN1 in the PAX2+ interneuron precursors (PIP), or in a polynucleotide of ASXL3, SHANK2, CACNA2D3, SLC6A1, PTEN, P2RX5, and/or UIMC1 in Purkinje cells (PC), or in a polynucleotide of PAX5 and/or ACHE in cerebellar nuclei (CN), and the neurological disease or condition comprises autism spectrum disorder;
wherein the genetic alteration is in a polynucleotide of DAB1, KIF26B, and/or ITPR1 in the PC, or in a polynucleotide of TBP in the PC, or in a polynucleotide of NOP56, FGF14, ATXN8OS, and/or PRKCG in the MLI, and the neurological disease or condition comprises spinocerebellar ataxia;
wherein the genetic alteration is in a polynucleotide of TEM231, B9D1, and/or NPHP1 in choroid plexus of the cerebellum-like organoid, and/or in a polynucleotide of CC2D2A in roof plate cells of the cerebellum-like organoid, and the neurological disease or condition comprises Joubert Syndrome; and
wherein the genetic alteration is in a polynucleotide of PTF1A and/or EBF2 in the PIP, and the neurological disease or condition comprises cerebellar malformation.


SEQUENCE LISTING (20 sequences)

SEQ ID NO: 1 - DNA, 19 bp, synthetic construct
  source 1..19: mol_type = other DNA
gagcgcagggcaccaaata

SEQ ID NO: 2 - DNA, 20 bp, synthetic construct
  source 1..20: mol_type = other DNA
cgagtcagttttgaccacgg

SEQ ID NO: 3 - DNA, 19 bp, synthetic construct
  source 1..19: mol_type = other DNA
ccggcgtgggtctactgta

SEQ ID NO: 4 - DNA, 20 bp, synthetic construct
  source 1..20: mol_type = other DNA
ggccgcttgtcctctttgtt

SEQ ID NO: 5 - DNA, 21 bp, synthetic construct
  source 1..21: mol_type = other DNA
cctgccctgtgagtctttaag

SEQ ID NO: 6 - DNA, 21 bp, synthetic construct
  source 1..21: mol_type = other DNA
gttcacttacagtctggtccc

SEQ ID NO: 7 - DNA, 21 bp, synthetic construct
  source 1..21: mol_type = other DNA
gacgagtcaaaggtggaagac

SEQ ID NO: 8 - DNA, 22 bp, synthetic construct
  source 1..22: mol_type = other DNA
gattgtcatccgagctgtagtc

SEQ ID NO: 9 - DNA, 20 bp, synthetic construct
  source 1..20: mol_type = other DNA
cgtcgctcgctgagtgcctg

SEQ ID NO: 10 - DNA, 23 bp, synthetic construct
  source 1..23: mol_type = other DNA
tgtcgagtgtgaaagcgtcgagg

SEQ ID NO: 11 - DNA, 20 bp, synthetic construct
  source 1..20: mol_type = other DNA
tcatagttccgtgagcgagc

SEQ ID NO: 12 - DNA, 20 bp, synthetic construct
  source 1..20: mol_type = other DNA
atccatgccattgtagccgt

SEQ ID NO: 13 - DNA, 22 bp, synthetic construct
  source 1..22: mol_type = other DNA
ggcttgcgccccgtagaaatgt

SEQ ID NO: 14 - DNA, 22 bp, synthetic construct
  source 1..22: mol_type = other DNA
aggagccaggtcaggtccgaac

SEQ ID NO: 15 - DNA, 19 bp, synthetic construct
  source 1..19: mol_type = other DNA
agaggacgacgttcactcg

SEQ ID NO: 16 - DNA, 21 bp, synthetic construct
  source 1..21: mol_type = other DNA
tcgggcaagttgattttcagt

SEQ ID NO: 17 - DNA, 22 bp, synthetic construct
  source 1..22: mol_type = other DNA
atagctgccgactaagcattga

SEQ ID NO: 18 - DNA, 19 bp, synthetic construct
  source 1..19: mol_type = other DNA
atccgtgcgaaggtgcttt

SEQ ID NO: 19 - DNA, 20 bp, synthetic construct
  source 1..20: mol_type = other DNA
accggcctcactcccacaca

SEQ ID NO: 20 - DNA, 20 bp, synthetic construct
  source 1..20: mol_type = other DNA
cgctcggtccaatggcctgg